(19) United States
(12) Patent Application Publication
(10) Pub. No.: US 2026/0337615 A1
(43) Pub. Date: Oct. 29, 2026

(54) Compositions and Methods for Reducing Ionizing Radiation-Induced Hematopoietic Stem Cell Damage

(71) Applicant: The Regents of the University of California, Oakland, CA (US)

(72) Inventors: Fyodor D. Urnov, Richmond, CA (US); Yunsi Refermat, Oakland, CA (US); Luke A. Gilbert, San Francisco, CA (US)

(21) Appl. No.: 19/103,515

(22) PCT Filed: Aug. 22, 2023
(86) PCT No.: PCT/US2023/072628

§ 371 Date: Feb. 12, 2025

Related U.S. Application Data
(60) Provisional application No. 63/400,164, filed on Aug. 23, 2022.

(51) Int. Cl. C12N 15/113; C07K 14/705; C12N 9/22; C12N 15/85

(52) U.S. Cl. CPC C12N 15/1138; C07K 14/70596; C12N 9/222; C12N 15/85; C07K 2319/09; C07K 2319/81; C12N 2310/20

(57) ABSTRACT

The present disclosure provides methods for reducing ionizing radiation-induced damage of cells such as hematopoietic stem cells (HSCs) and hematopoietic progenitor cells (HPCs). The methods comprise introducing into HSCs and/or HPCs a fusion protein comprising (a) a zinc finger DNA binding polypeptide with an alpha-helix recognition domain configured to bind to a target nucleotide sequence in a target nucleic acid, and (b) an epigenome modifying polypeptide.

### CROSS-REFERENCE

This application claims the benefit of U.S. Provisional Patent Application No. 63/400,164, filed Aug. 23, 2023, which application is incorporated herein by reference in its entirety.

### STATEMENT REGARDING FEDERALLY SPONSORED RESEARCH

This invention was made with government support under Grant No. HR0011-19-2-0007 awarded by the Department of Defense Advanced Research Projects Agency. The government has certain rights in the invention.

### INCORPORATION-BY-REFERENCE OF MATERIAL ELECTRONICALLY SUBMITTED

A Sequence Listing is provided herewith as a Sequence Listing XML, “BERK-476WO_SEQ_LIST” created on Aug. 15, 2023 and having a size of 123,156 bytes. The contents of the Sequence Listing XML are incorporated by reference herein in their entirety.

### INTRODUCTION

Hematopoiesis, the process of blood cell formation, occurs during embryonic development and throughout adulthood to produce and replenish blood cells. In adults, hematopoiesis is mainly sustained by hematopoietic stem cells (HSC) residing in the bone marrow. HSCs are critical for lifelong blood production and HSCs have the capacity to self-renew, or generate daughter stem cells, while still contributing to the pool of differentiating cells. HSCs sit atop a hierarchy of progenitors that become progressively restricted to several or single lineages. These progenitors yield blood precursors devoted to unilineage differentiation and production of mature blood cells, including red blood cells, megakaryocytes, myeloid cells (monocyte/macrophage and neutrophil), and lymphocytes. In children, hematopoiesis occurs in the marrow of the long bones such as the femur and tibia. In adults, it occurs mainly in the pelvis, cranium, vertebrae, and sternum. However, maturation, activation, and some proliferation of certain hematopoietic cells occurs in extramedullary hematopoietic niches such as the peripheral blood, spleen, liver, thymus, and lymph nodes.

Radiation is often used to treat malignancies such as various forms of cancer. However, it is well established that exposure to ionizing radiation causes a drastic deficit to bone marrow cell populations and even sub-lethal doses may lead to irreparable tissue damage of the hematopoietic stem and progenitor cells residing in the bone marrow and other hematopoietic niches. Bone marrow toxicity from radiation results from damage to HSC as well as bone microenvironment. Exposure to high doses of ionizing radiation or chemotherapy will lead to bone marrow failure and ultimately may lead to death. However, high doses of ionizing radiation as well as chemotherapy are often beneficial for pursuing medical treatment. Many patients with cancers undergo elective radiation therapy or chemotherapy; in particular, patients with tumors of organs such as the ovaries, the cervix, and the uterus may also undergo localized radiotherapy that exposes the bone marrow and the HSCs that reside therein to ionizing radiation. The resulting impairment of hematopoiesis represents a dose-limiting toxicity of radiotherapy.

### SUMMARY

The present disclosure provides methods for reducing ionizing radiation-induced damage of cells such as hematopoietic stem cells (HSCs) and hematopoietic progenitor cells (HPCs). The methods comprise introducing into HSCs and/or HPCs a fusion protein comprising (a) a zinc finger DNA binding polypeptide with an alpha-helix recognition domain configured to bind to a target nucleotide sequence in a target nucleic acid, and (b) an epigenome modifying polypeptide.

### BRIEF DESCRIPTION OF THE DRAWINGS

The invention may be best understood from the following detailed description when read in conjunction with the accompanying drawings. Included in the drawings are the following figures:

FIG. 1A-1B present amino acid sequences of ZFPOFF polypeptides according to certain embodiments of the disclosure.

FIG. 2A-2B depict ZFP amino acid sequence structure using a CD55-targeting ZFP (FIG. 2A) and a DNA sequence encoding the same (FIG. 2B).

FIG. 3A-3C depict exemplary nucleic acid sequences according to certain embodiments of the present disclosure.

FIG. 4A-4D depict representative maps of ZFPOFF cloning vectors according to certain embodiments of the disclosure.

FIG. 5A-5C depict various ZFPOFF constructs according to certain embodiments of the invention.

FIG. 6A-6F present qualitative graphs depicting repression of CD55 using different ZFPOFF constructs in a human leukemia cancer cell line, K562.

FIG. 7A-7B depict DNA sequences encoding Dnmt3A and Dnmt3L DNA methyltransferase domains, a multi-cloning site (MCS) in frame with translation, a blue fluorescent protein marker (BFP) and a KRAB repressor domain cloned into the pVAX1 vector.

FIG. 8A-8B present a comparison between ZFPOFF, ZFP-KRAB and CRISPRoff constructs in K562.

FIG. 9A-9B present a comparison between the effects of CD55 targeting by ZFPOFF and CRISPRoff V2.4 plasmid constructs.

FIG. 10A-10B present a comparison between the effects of CD55 targeting by ZFPOFF and CRISPRoff V2.4 mRNAs.

FIG. 11A-11C present CD55-PE histograms of human hematopoietic stem and progenitor cells (HSPCs) electroporated with ZFPOFF or CRISPRoff mRNA.

FIG. 12 presents an ex vivo radioprotection experimental outline.

FIG. 13A-13F depict the screening of gene targets screened advanced to an ex vivo radioprotection experiment in HSPCs.

FIG. 14A-14D present depictions of the viability of HSPCs in which TP53 is repressed.

FIG. 15A-15D present depictions of the viability of HSPCs in which AUNIP is repressed.

FIG. 16A-16D present depictions of the viability of HSPCs in which PPP6C is repressed.

FIG. 17A-17D present depictions of the viability of HSPCs in which BAX is repressed.

FIG. 18A-18D present depictions of the viability of HSPCs in which CD81 is repressed.

FIG. 19A-19B depict the optimization of ZFPoff mRNA dose.

FIG. 20A-20D present the results of a pilot study using cell surface marker genes with respect to ZFPOFF programmable gene modulator multiplexing.

FIG. 21A-21B present an assessment of the effects of different ZFPOFF mRNA doses on HSPC stem cell growth.

FIG. 22A-22D present a time course for establishing ZFPoff mRNA dose for positive control and lead gene targets.

FIG. 23A-23B depict the optimization of ZFPoff mRNA dose using cell surface marker gene targets in primary human T cells.

FIG. 24A-24B depict the optimization of ZFPoff mRNA dose and multiplexing using cell surface marker gene targets in primary human T cells.

FIG. 25 shows the sensitivity of certain cell lines to CD81 knockout.

### DEFINITIONS

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

The term “oligonucleotide” refers to a polynucleotide of between 3 and 100 nucleotides of single- or double-stranded nucleic acid (e.g., DNA, RNA, or a modified nucleic acid). However, for the purposes of this disclosure, there is no upper limit to the length of an oligonucleotide. Oligonucleotides are also known as “oligomers” or “oligos” and can be isolated from genes, transcribed (in vitro and/or in vivo), or chemically synthesized. The terms “polynucleotide” and “nucleic acid” should be understood to include, as applicable to the embodiments being described, single-stranded (such as sense or antisense) and double-stranded polynucleotides.

A “gene,” for the purposes of the present disclosure, includes a DNA region encoding a gene product (e.g., a polypeptide or a polynucleotide), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Zinc finger proteins” (ZFPs) are proteins that bind to DNA, RNA and/or protein, in a sequence-specific manner, by virtue of a metal stabilized domain known as a zinc finger. See, for example, Miller et al. (1985) EMBO J. 4:1609-1614; Rhodes et al. (1993) Sci. Amer. 268(2):56-65; and Klug (1999) J. Mol. Biol. 293:215-218. ZFPs are commonly found in transcription factors, and to date, over 10,000 zinc finger sequences have been identified in several thousand known or putative transcription factors.

A “zinc finger DNA binding protein” is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; 6,534,261; and 6,785,613; see, also WO 98/53058; WO 98/53059; WO 98/53060; WO 02/016536 and WO 03/016496; and U.S. Pat. Nos. 6,746,838; 6,866,997; and 7,030,215.

A “target” nucleic acid is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease.

“Binding” as used herein (e.g. with reference to an RNA-binding domain of a polypeptide, binding to a target nucleic acid, and the like) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (Kd) of less than 10−6 M, less than 10−7 M, less than 10−8 M, less than 10−9 M, less than 10−10 M, less than 10−11 M, less than 10−12 M, less than 10−13 M, less than 10−14 M, or less than 10−15 M. “Affinity” refers to the strength of binding, increased binding affinity being correlated with a lower Kd.

By “binding domain” it is meant a protein domain that is able to bind non-covalently to another molecule. A binding domain can bind to, for example, an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence or a coding sequence, and/or regulate translation of an encoded polypeptide.

As used herein, a “promoter sequence” is a DNA regulatory region capable of binding RNA polymerase and initiating transcription of a downstream (3′ direction) coding or non-coding sequence. For purposes of the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present disclosure.

The term “naturally-occurring” or “unmodified” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is wild type (and naturally occurring).

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic: Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Alternatively, DNA sequences encoding RNA that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “vector” or “expression vector” is a replicon, such as plasmid, phage, virus, or cosmid, to which another DNA segment, i.e. an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression.

The terms “recombinant expression vector,” or “DNA construct” are used interchangeably herein to refer to a DNA molecule comprising a vector and one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to anon-heat-shocked cell. An exogenous molecule can comprise, for example, a functioning version of a malfunctioning endogenous molecule or a malfunctioning version of a normally-functioning endogenous molecule.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP DNA-binding domain and epigenome modifying domain) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

“Modulation” of gene expression refers to a change in the activity of a gene. Modulation of expression can include, but is not limited to, gene activation and gene repression.

Hematopoietic stem and progenitor cells include, but are not limited to, long term hematopoietic stem cells (LT-HSCs), short term hematopoietic stem cells (ST-HSCs), multipotent progenitors (MPPs), common myeloid progenitors (CMPs), common lymphoid progenitors (CLPs), granulocyte-monocyte progenitors (GMPs), and megakaryocyte-erythroid progenitors (MEPs).

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a zinc finger DNA binding polypeptide” includes a plurality of such polypeptides and reference to “the DNA methyltransferase” includes reference to one or more DNA methyltransferases and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods for reducing ionizing radiation-induced damage of cells such as hematopoietic stem cells (HSCs) and hematopoietic progenitor cells (HPCs). The methods comprise introducing into HSCs and/or HPCs: (a) a fusion protein comprising (i) a zinc finger DNA binding polypeptide with an alpha-helix recognition domain configured to bind to a target nucleotide sequence in a target nucleic acid, and (ii) an epigenome modifying polypeptide; or (b) a nucleic acid comprising a nucleotide sequence encoding such a fusion protein. A fusion protein comprising a zinc finger DNA binding polypeptide and an epigenome modifying polypeptide is also referred to herein as “ZFPoff”.

### Methods

The present disclosure provides a method for reducing the effect of ionizing radiation exposure on hematopoietic stem cells and/or progenitor cells (HPCs) in an individual. In some cases, the method comprises introducing into HSCs and/or HPCs a fusion protein comprising (a) a zinc finger DNA binding polypeptide with an alpha-helix recognition domain configured to bind to a target nucleotide sequence in a target nucleic acid, and (b) an epigenome modifying polypeptide. In some cases, the method comprises introducing into HSCs and/or HPCs a nucleic acid comprising a nucleotide sequence encoding a fusion protein comprising (a) a zinc finger DNA binding polypeptide with an alpha-helix recognition domain configured to bind to a target nucleotide sequence in a target nucleic acid, and (b) an epigenome modifying polypeptide. In some cases, the target nucleic acid is selected from: CD81, PPP6C, BAX, and AUNIP. Suitable fusion proteins and nucleic acids are described in detail below. In some cases, the method comprises administering to an individual in need thereof (e.g., an individual who has undergone radiation treatment for cancer; an individual who is about to undergo radiation treatment for cancer; an individual who has been exposed to ionizing radiation; an individual who is about to be exposed to ionizing radiation; etc.) one of: a) a fusion protein comprising (i) a zinc finger DNA binding polypeptide with an alpha-helix recognition domain configured to bind to a target nucleotide sequence in a target nucleic acid, and (i) an epigenome modifying polypeptide; b) a nucleic acid comprising a nucleotide sequence encoding the fusion protein; or c) a recombinant expression vector comprising a nucleotide sequence encoding the fusion protein.

In some cases, the ionizing radiation is administered for the treatment of a cancer, e.g., where the cancer is small cell lung cancer, triple-negative breast cancer, human papilloma virus (HPV)-positive head and neck cancer, retinoblastoma, retinoblastoma protein (Rb)-negative bladder cancer, retinoblastoma protein (Rb)-negative prostate cancer, osteosarcoma, ovarian cancer, uterine cancer, or cervical cancer.

In some cases, the fusion protein silences the target nucleic acid sequence in the cell by methylating a chromatin containing the target nucleic acid sequence and/or by introducing repressive chromatin marks to a chromatin containing the target nucleic acid sequence. Methylating a chromatin means that DNA is methylated at the C nucleotide of CG sequences found in CpG islands (i.e., adding methyl marks at the C nucleotide of CG DNA sites found in CpG islands). In aspects, the sequence that is within about 3000 base pairs of the target nucleic acid sequence is methylated. In aspects, the sequence that is within about 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, or 100 base pairs of the target nucleic acid sequence is methylated.

The term “repressive chromatin markers” as used herein refers to modifications made to the chromatin that result in silencing (e.g., decreasing or inhibiting of transcription) of the target nucleic acid sequence (e.g., a gene). Examples of repressive chromatin markers include, but are not limited to, mono-, di-, and/or tri-methylation, acetylation/deacetylation, phosphorylation, and ubiquitination of histones (e.g., H3K9, H3K27, H3K79, H2BK5).

In embodiments, the method has a specificity that is 2-fold higher than a specificity to a non-target nucleic acid sequence. In aspects, the method has a specificity that is at least 2-fold (e.g., 2-, 3-, 4-, 5-, 6-, 7-, 8-, 9-, 10-, 15-, 20-, 25-fold) higher than a specificity to a non-target nucleic acid sequence. Methods for determining specificity are well known in the art and include, but are not limited to, RNA-seq, bisulfite sequencing, chromatin immunoprecipitation, flow cytometry, and qPCR. Thus, in aspects, specificity is determined by RNA-seq. In aspects, specificity is determined by bisulfite sequencing. In aspects, specificity is determined by chromatin immunoprecipitation. In aspects, specificity is determined by flow cytometry. In aspects, specificity is determined by quantitative polymerase chain reaction (qPCR).

The cell in which the target gene is silenced is an HSC or an HPC.

In some cases, the methods comprise administering to an individual in need thereof (a) a fusion protein comprising (i) a zinc finger DNA binding polypeptide with an alpha-helix recognition domain configured to bind to a target nucleotide sequence in a target nucleic acid, and (ii) an epigenome modifying polypeptide; or (b) a nucleic acid comprising a nucleotide sequence encoding such a fusion protein. In some cases, the individual has undergone ionizing radiation (IR) treatment for a cancer. In some cases, the individual is about to undergo IR treatment for a cancer. In some cases, the individual is exposed to IR at least 5 times a week, at least 4 times a week, at least 3 times a week, at least 2 times a week, at least 1 time a week, at least 3 times a month, at least 2 times a month, or at least 1 time a month, for the treatment of cancer. In some cases, a fusion protein, or a nucleic acid encoding the fusion protein, is administered to an individual within 72 hours, within 36 hours, within 24 hours, within 12 hours, within 8 hours, within 4 hours, within 2 hours, or within 1 hour, of IR treatment for a cancer. In some cases, a fusion protein, or a nucleic acid encoding the fusion protein, is administered to an individual from about 72 hours to about 36 hours, from about 36 hours to about 24 hours, from about 24 hours to about 12 hours, from about 12 hours to about 8 hours, from about 8 hours to about 4 hours, from about 4 hours to about 2 hours, or from about 2 hours to about 1 hour, before IR treatment for a cancer. In some cases, a fusion protein, or a nucleic acid encoding the fusion protein, is administered to an individual from about 72 hours to about 36 hours, from about 36 hours to about 24 hours, from about 24 hours to about 12 hours, from about 12 hours to about 8 hours, from about 8 hours to about 4 hours, from about 4 hours to about 2 hours, or from about 2 hours to about 1 hour, after IR treatment for a cancer.

In some cases, the methods comprise administering to an individual in need thereof (a) a fusion protein comprising (i) a zinc finger DNA binding polypeptide with an alpha-helix recognition domain configured to bind to a target nucleotide sequence in a target nucleic acid, and (ii) an epigenome modifying polypeptide; or (b) a nucleic acid comprising a nucleotide sequence encoding such a fusion protein. In some cases, the individual has been exposed to ionizing radiation, will be exposed to ionizing radiation, or is at risk of incurring exposure to ionizing radiation as the result of radiological agent exposure. For example, in some cases, the individual has been exposed to ionizing radiation, will be exposed to ionizing radiation, or is at risk of incurring exposure to ionizing radiation as the result of radiological agent exposure during warfare, a radiological terrorist attack, an industrial accident, or space travel. In some cases, the individual is exposed to IR at least 5 times a week, at least 4 times a week, at least 3 times a week, at least 2 times a week, at least 1 time a week, at least 3 times a month, at least 2 times a month, or at least 1 time a month. In some cases, a fusion protein, or a nucleic acid encoding the fusion protein, is administered to an individual within 72 hours, within 36 hours, within 24 hours, within 12 hours, within 8 hours, within 4 hours, within 2 hours, or within 1 hour, of IR exposure. In some cases, a fusion protein, or a nucleic acid encoding the fusion protein, is administered to an individual from about 72 hours to about 36 hours, from about 36 hours to about 24 hours, from about 24 hours to about 12 hours, from about 12 hours to about 8 hours, from about 8 hours to about 4 hours, from about 4 hours to about 2 hours, or from about 2 hours to about 1 hour, before IR exposure. In some cases, a fusion protein, or a nucleic acid encoding the fusion protein, is administered to an individual from about 72 hours to about 36 hours, from about 36 hours to about 24 hours, from about 24 hours to about 12 hours, from about 12 hours to about 8 hours, from about 8 hours to about 4 hours, from about 4 hours to about 2 hours, or from about 2 hours to about 1 hour, after IR exposure.

In some cases, an effective amount of the fusion protein, or a nucleic acid encoding the fusion protein, is administered to an individual in need thereof. In some cases, an effective amount of (a) a fusion protein comprising (i) a zinc finger DNA binding polypeptide with an alpha-helix recognition domain configured to bind to a target nucleotide sequence in a target nucleic acid, and (ii) an epigenome modifying polypeptide; or (b) a nucleic acid comprising a nucleotide sequence encoding such a fusion protein, is an amount that provides for maintenance of at least 50%, at least 60%, at least 65%, at least 70%, at least 75%, at least 80%, at least 85%, or at least 90%, of healthy HSCs or HPCs that were present in the individual before IR exposure.

Individuals suitable for treatment using a method of the present disclosure include an individual has been exposed to ionizing radiation, will be exposed to ionizing radiation, or is at risk of incurring exposure to ionizing radiation as the result of radiological agent exposure during warfare, a radiological terrorist attack, an industrial accident, or space travel.

Individuals suitable for treatment using a method of the present disclosure include an individual who have been exposed to, or is scheduled to be exposed to, ionizing radiation when undergoing therapeutic irradiation for the treatment of proliferative disorders. Such disorders include cancerous and non-cancer proliferative diseases. The compounds are effective in protecting healthy hematopoietic stem/progenitor cells during therapeutic irradiation of a broad range of tumor types, including but not limited to the following: breast, prostate, ovarian, skin, lung, colorectal, brain (i.e., glioma) and renal. Such cancers can include, but are not limited to, small cell lung cancer, retinoblastoma, HPV positive malignancies like cervical cancer and certain head and neck cancers, MYC amplified tumors such as certain classes of Rb-positive Burkitts Lymphoma, and triple negative breast cancer; sarcomas; non-small cell lung carcinoma; melanoma; pancreatic cancer; leukemias; lymphomas; brain cancer; colon cancer; prostate cancer; ovarian cancer; uterine cancer; thyroid and other endocrine tissue cancers; salivary cancers; head and neck cancers; thymic carcinomas; kidney cancers; bladder cancer; testicular cancers.

### Fusion Proteins

Aspects of the present disclosure include fusion proteins. Fusion proteins of interest (also referred to herein as “ZFPOFF”) include a zinc finger DNA binding polypeptide comprising an alpha-helix recognition domain configured to bind to a target nucleotide sequence in a target nucleic acid, and an epigenome modifying polypeptide. In certain cases, the subject fusion proteins can turn off genes permanently (e.g., irreversibly) and reversibly in mammalian cells. The fusion protein can be directed to a specific site in a mammalian genome using a polynucleotide complementary to a target nucleic acid sequence (e.g., DNA sequence) and that further includes a sequence (i.e., binding sequence) capable of binding the fusion protein. Once properly positioned and without intending to be bound by a theory, the fusion protein adds DNA methylation and/or repressive chromatin marks to the target nucleic acid, resulting in gene silencing that is inheritable across subsequent cell divisions. In this way, the fusion protein can perform epigenome editing that bypasses the need to generate DNA double-strand breaks in the host genome, making it a safe and reversible way of manipulating the genome of a living organism.

### Zinc Finger DNA Binding Polypeptides

A zinc finger binding polypeptide of the disclosure comprises one or more zinc fingers. See, e.g., Miller et al. (1985) EMBO J. 4:1609-1614; Rhodes (1993) Scientific American February: 56-65; U.S. Pat. No. 6,453,242, the disclosures of which are incorporated by reference herein. Zinc fingers for use in the subject zinc finger binding polypeptide may vary, as desired. In some embodiments, zinc fingers range in length from 28 to 32 amino acids. In certain cases, one or more zinc fingers is 30 amino acids in length. In some instances, a zinc finger domain (motif) contains two beta sheets (held in a beta turn which contains two cysteine residues) and an alpha helix (containing two histidine residues), which are held in a particular conformation through coordination of a zinc atom by the two cysteines and the two histidines. General discussions of zinc finger proteins may be found in U.S. Pat. Nos. 8,313,925; 8,399,218; 8,871,905; 9,234,016; 9,624.509; and 10,662,434; the disclosures of which are herein incorporated by reference in their entirety.

Zinc fingers include both canonical C₂H₂ zinc fingers (i.e., those in which the zinc ion is coordinated by two cysteine and two histidine residues) and non-canonical zinc fingers such as, for example, C₃H zinc fingers (those in which the zinc ion is coordinated by three cysteine residues and one histidine residue), C₁H₃ zinc fingers and C₄ zinc fingers (those in which the zinc ion is coordinated by four cysteine residues). See also WO 02/057293. In some embodiments, the zinc finger DNA binding polypeptide includes a canonical C₂H₂ zinc finger. In other embodiments, the zinc finger DNA binding polypeptide includes a non-canonical zinc finger.

Zinc finger binding domains can be engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261. Additional design methods are disclosed, for example, in U.S. Pat. Nos. 6,746,838; 6,785,613; 6,866,997; and 7,030,215.

Since an individual zinc finger binds to a three-nucleotide (i.e., triplet) sequence (or a four-nucleotide sequence which can overlap, by one nucleotide, with the four-nucleotide binding site of an adjacent zinc finger), the length of a sequence to which a zinc finger binding polypeptide is engineered to bind (e.g., a target sequence) will determine the number of zinc fingers in an engineered zinc finger binding domain. For example, for ZFPs in which the finger motifs do not bind to overlapping subsites, a six-nucleotide target sequence is bound by a two-finger binding domain; a nine-nucleotide target sequence is bound by a three-finger binding domain, etc. As noted herein, binding sites for individual zinc fingers (i.e., subsites) in a target site need not be contiguous, but can be separated by one or several nucleotides, depending on the length and nature of the amino acid sequences between the zinc fingers (i.e., the inter-finger linkers) in a multi-finger binding domain.

The subject zinc finger binding polypeptide may include any convenient number of zinc fingers. In certain cases, the number of zinc fingers in the zinc finger binding polypeptide ranges from 1 to 5, such as 2 to 3. In some embodiments, the zinc finger binding polypeptide includes 1 zinc finger. In other embodiments, the zinc finger binding polypeptide includes 2 zinc fingers. In still other embodiments, the zinc finger binding polypeptide includes 3 zinc fingers. In yet other embodiments, the zinc finger binding polypeptide includes 4 zinc fingers.

In some embodiments where the zinc finger binding polypeptide is a multi-finger zinc finger binding polypeptide, adjacent zinc fingers are separated by amino acid linker. Amino acid linker sequences of interest may include 4-6 amino acids (e.g., 5 amino acids; so-called “canonical” inter-finger linkers) or, alternatively, by one or more non-canonical linkers. Non-canonical linkers may be found in, e.g., U.S. Pat. Nos. 6,453,242 and 6,534,261. For engineered zinc finger binding domains comprising more than three fingers, insertion of longer (“non-canonical”) inter-finger linkers between certain of the zinc fingers may be preferred as it may increase the affinity and/or specificity of binding by the binding domain. See, for example, U.S. Pat. No. 6,479,626 and WO 01/53480, Accordingly, multi-finger zinc finger binding domains can also be characterized with respect to the presence and location of non-canonical inter-finger linkers. For example, a six-finger zinc finger binding domain comprising three fingers (joined by two canonical inter-finger linkers, A long linker and three additional fingers (joined by two canonical inter-finger linkers) is denoted a 2×3 configuration. Similarly, a binding domain comprising two fingers (with a canonical linker therebetween), a long linker and two additional fingers (joined by a canonical linker) is denoted a 2×2 protein. A protein comprising three, two-finger units (in each of which the two fingers are joined by a canonical linker), and in which each two-finger unit is joined to the adjacent two finger unit by a long linker, is referred to as a 3×2 protein.

The presence of a long or non-canonical inter-finger linker between two adjacent zinc fingers in a multi-finger binding domain often allows the two fingers to bind to subsites which are not immediately contiguous in the target sequence. Accordingly, there can be gaps of one or more nucleotides between subsites in a target site; i.e., a target site can contain one or more nucleotides that are not contacted by a zinc finger. For example, a 2×2 zinc finger binding domain can bind to two six-nucleotide sequences separated by one nucleotide, i.e., it binds to a 13-nucleotide target site. See also Moore et al. (2001a) Proc. Natl. Acad. Sci. USA 98:1432-1436; Moore et al. (2001b) Proc. Natl. Acad. Sci. USA 98:1437-1441 and WO 01/53480.

Zinc finger DNA binding polypeptides of the disclosure include one or more alpha-helix recognition domains. The subject alpha helix recognition domains are configured to make sequence-specific contacts to DNA bases. In some embodiments, residues of the alpha-helix recognition domain contact 3 or more bases, such as 4 or more bases, such as 5 or more bases, and including 6 or more bases. As such, alpha-helix recognition domains described herein may be configured to bind to a target nucleotide sequence in a target nucleic acid. Any suitable target nucleic acid may serve as the target nucleic acid of the subject methods. In some cases, the alpha-helix recognition domain is configured to recognize target nucleic acid encoding a cancer-associated polypeptide. As such, the subject target nucleic acid may be associated with an oncogene, proto-oncogene, or the like. Target nucleic acids include, but are not limited to, AUNIP, PPP6C, CD81, and BAX. In additional cases, the alpha-helix recognition domain is configured to bind to a nucleotide sequence in a target CD81 nucleic acid. In select cases, the alpha-helix recognition domain is configured to bind to a nucleotide sequence in a target BAX nucleic acid. In certain cases, the alpha-helix recognition domain is configured to bind to a nucleotide sequence in a target PPP6C nucleic acid. In other cases, the alpha-helix recognition domain is configured to bind to a nucleotide sequence in a target AUNIP nucleic acid.

Exemplary amino acid sequences that may be employed in the subject zinc finger DNA binding polypeptides are presented in Table 1, along with exemplary DNA coding sequences associated therewith. In some cases, a zinc finger DNA binding polypeptide (of the subject fusion proteins, compositions and methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) to any one of the ZFP amino acid sequences presented in Table 1. In certain embodiments, the zinc finger DNA binding polypeptide includes an amino acid sequence having 70% or more sequence identity (e.g., 75% or more, 80% or more, 85% or more, 90% or more, 95% or more, 97% or more, 98% or more, 99% or more, or 100% sequence identity) to any one of the ZFP amino acid sequences presented in Table 1. In some cases, a zinc finger DNA binding polypeptide (of the subject fusion proteins, compositions and methods) may be encoded by any one of the ZFP DNA sequences provided in Table 1.

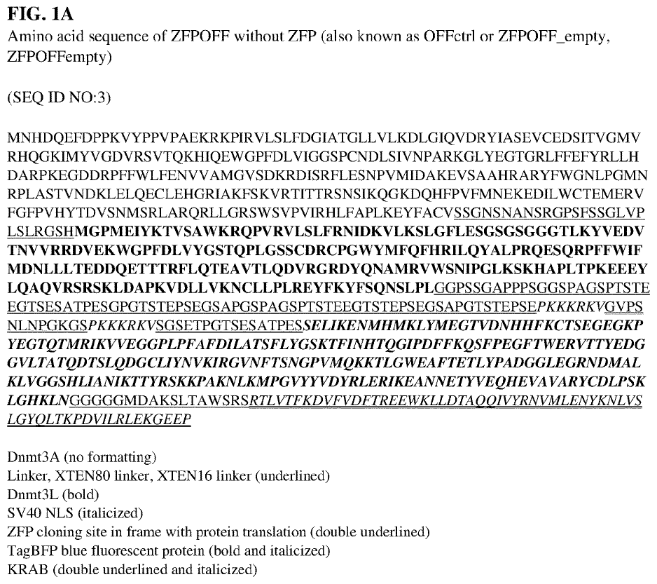

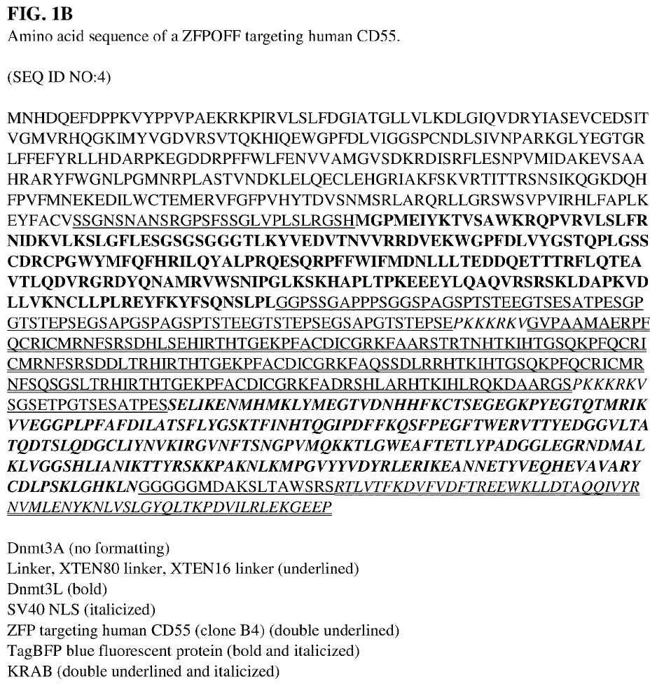

In some embodiments, the nucleic acids encoding fusion proteins include a ZFP cloning site. In embodiments, such fusion proteins do not include a zinc finger DNA binding polypeptide, e.g., do not comprise an amino acid such as those presented below with respect to Table 1. As discussed herein, such a fusion protein may be referred to as “OFFctrl”, “ZFPOFF_empty” or ZFPOFFempty”. In some embodiments, the nucleic acid encoding a fusion protein having a ZFP cloning site (i.e. does not include a zinc finger DNA binding polypeptide) where the fusion protein does not exhibit an epigenome modifying property. The fusion protein encoded by a nucleic acid comprising a ZFP cloning site may, in select instances, be employed as a control when analyzed with respect to a fusion protein having a zinc finger DNA binding polypeptide of the present disclosure. In some cases, a fusion protein encoded by a nucleic acid including a ZFP cloning site includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) to GVPSNLNPGKGS (SEQ ID NO: 1). In certain embodiments, a fusion protein encoded by a nucleic acid including a ZFP cloning site includes an amino acid sequence having 70% or more sequence identity (e.g., 75% or more, 80% or more, 85% or more, 90% or more, 95% or more, 97% or more, 98% or more, 99% or more, or 100% sequence identity) to GVPSNLNPGKGS (SEQ ID NO: 1). In some cases, a polynucleotide encoding a fusion protein including a ZFP cloning site may have a nucleic acid sequence of ggggtaccgtcaaatttaaatcccgggaagggatcc (SEQ ID NO: 55). An exemplary amino acid sequence of a ZFPOFF_empty protein is shown in FIG. 1A.

TABLE 1
Exemplary Zinc Finger DNA Binding Polypeptides

| ZFP | Alt | ZFP Amino | |
|---|---|---|---|
| Name | Name | Acid Sequence | ZFP DNA Sequence |
| CD81_ | CD81_ | GVPAAMAERPFQCRICMR | ggggtacccgccgctatggctgagaggcccttccagtgtcgaatctgcatgcgtaac |
| 8 | A | NFSDDWNLSQHIRTHTGEK | ttcagtgacgactggaacctgtcccagcacatccgcacccacaccggcgagaagc |
| | | PFACDICGRKFARSANLTR | cttttgcctgtgacatttgtgggaggaaatttgcccgctccgccaacctgacccgccat |
| | | HTKIHTGSQKPFQCRICMR | accaagatacacacgggcagccaaaagcccttccagtgtcgaatctgcatgcgtaa |
| | | NFSHKQHRDAHIRTHTGEK | cttcagtcacaagcagcaccgcgacgcccacatccgcacccacaccggcgagaa |
| | | PFACDICGRKFARSANLTR | gccttttgcctgtgacatttgtgggaggaaatttgcccgctccgccaacctgacccgc |
| | | HTKIHTGSQKPFQCRICMR | cataccaagatacacacgggatctcagaagcccttccagtgtcgaatctgcatgcgt |
| | | NFSRSDDLTRHIRTHTGEK | aacttcagtcgctccgacgacctgacccgccacatccgcacccacaccggcgaga |
| | | PFACDICGRKFAMSYQLGR | agccttttgcctgtgacatttgtgggaggaaatttgccatgtcctaccagctgggccgc |
| | | HTKIHLRQKDAARGS (SEQ | cataccaagatacacctgcgccaaaaagatgcggcccggggatcc (SEQ ID |
| | | ID NO: 11) | NO: 65) |
| CD81_ | CD81_ | GVPAAMAERPFQCRICMR | ggggtacccgccgctatggctgagaggcccttccagtgtcgaatctgcatgcgtaac |
| 18 | B | NFSDRSDLSRHIRTHTGEK | ttcagtgaccgctccgacctgtcccgccacatccgcacccacaccggcgagaagcc |
| | | PFACDICGRKFARRTDLRR | ttttgcctgtgacatttgtgggaggaaatttgcccgccgcaccgacctgcgccgccat |
| | | HTKIHTGSQKPFQCRICMR | accaagatacacacgggcagccaaaagcccttccagtgtcgaatctgcatgcgtaa |
| | | NFSRSDDLTRHIRTHTGEK | cttcagtcgctccgacgacctgacccgccacatccgcacccacaccggcgagaag |
| | | PFACDICGRKFADRSTLAR | ccttttgcctgtgacatttgtgggaggaaatttgccgaccgctccaccctggcccgcc |
| | | HTKIHTGSQKPFQCRICMR | ataccaagatacacacgggatctcagaagcccttccagtgtcgaatctgcatgcgta |
| | | NFSRSAHLSRHIRTHTGEK | acttcagtcgctccgcccacctgtcccgccacatccgcacccacaccggcgagaag |
| | | PFACDICGRKFADRSHLAR | ccttttgcctgtgacatttgtgggaggaaatttgccgaccgctcccacctggcccgcc |
| | | HTKIHLRQKDAARGS (SEQ | ataccaagatacacctgcgccaaaaagatgcggcccggggatcc (SEQ ID |
| | | ID NO: 12) | NO: 66) |
| CD81_ | CD81_ | GVPAAMAERPFQCRICMR | ggggtacccgccgctatggctgagaggcccttccagtgtcgaatctgcatgcgtaac |
| 19 | C | NFSRSADLTRHIRTHTGEK | ttcagtcgctccgccgacctgacccgccacatccgcacccacaccggcgagaagc |
| | | PFACDICGRKFADRSHLAR | cttttgcctgtgacatttgtgggaggaaatttgccgaccgctcccacctggcccgccat |
| | | HTKIHTGSQKPFQCRICMR | accaagatacacacgggcagccaaaagcccttccagtgtcgaatctgcatgcgtaa |
| | | NFSYKCCLSDHIRTHTGEK | cttcagttacaagtgttgcctgtccgaccacatccgcacccacaccggcgagaagcc |
| | | PFACDICGRKFARSANLTR | ttttgcctgtgacatttgtgggaggaaatttgcccgctccgccaacctgacccgccata |
| | | HTKIHTGSQKPFQCRICMR | ccaagatacacacgggatctcagaagcccttccagtgtcgaatctgcatgcgtaactt |
| | | NFSRSDTLSQHIRTHTGEKP | cagtcgtagtgacaccctgagccagcacatccgcacccacacaggcgagaagcct |
| | | FACDICGRKFVARSTRITHT | tttgcctgtgacatttgtgggagaaaatttgtcgccaggagcacccgcataacccata |
| | | KIHLRQKDAARGS (SEQ ID | ccaagatacacctgcgccaaaaagatgcggcccggggatcc (SEQ ID |
| | | NO: 13) | NO: 67) |
| CD81_ | CD81_ | GVPAAMAERPFQCRICMR | ggggtacccgccgctatggctgagaggcccttccagtgtcgaatctgcatgcgtaac |
| 2 | D | NFSQSSDLSRHIRTHTGEKP | ttcagtcagtcctccgacctgtcccgccacatccgcacccacaccggcgagaagcc |
| | | FACDICGRKFADRSVLRRH | ttttgcctgtgacatttgtgggaggaaatttgccgaccgctccgtgctgcgccgccata |
| | | TKIHTGSQKPFQCRICMRN | ccaagatacacacgggcagccaaaagcccttccagtgtcgaatctgcatgcgtaact |
| | | FSDNRDLSTHIRTHTGEKPF | tcagtgacaaccgcgacctgtccacccacatccgcacccacaccggcgagaagcc |
| | | ACDICGRKFADRSNLTRHT | ttttgcctgtgacatttgtgggaggaaatttgccgaccgctccaacctgacccgccata |
| | | KIHTGSQKPFQCRICMRNF | ccaagatacacacgggatctcagaagcccttccagtgtcgaatctgcatgcgtaactt |
| | | SDRSHLSRHIRTHTGEKPF | cagtgaccgctcccacctgtcccgccacatccgcacccacaccggcgagaagcctt |
| | | ACDICGRKFADRSHLARHT | ttgcctgtgacatttgtgggaggaaatttgccgaccgctcccacctggcccgccatac |
| | | KIHLRQKDAARGS (SEQ ID | caagatacacctgcgccaaaaagatgggcccggggatcc (SEQ ID |
| | | NO: 14) | NO: 68) |
| AUNIP_ | AUNIP_ | GVPAAMAERPFQCRICMR | ggggtacccgccgctatggctgagaggcccttccagtgtcgaatctgcatgcgtaac |
| 5 | A | NFSDRSHLTRHIRTHTGEK | ttcagtgaccgctcccacctgacccgccacatccgcacccacaccggcgagaagc |
| | | PFACDICGRKFARSDHLSR | cttttgcctgtgacatttgtgggaggaaatttgcccgctccgaccacctgtcccgccat |
| | | HTKIHTGSQKPFQCRICMR | accaagatacacacgggcagccaaaagcccttccagtgtcgaatctgcatgcgtaa |
| | | NFSQSGNLARHIRTHTGEK | cttcagtcagtccggcaacctggcccgccacatccgcacccacaccggcgagaag |
| | | PFACDICGRKFAATCCLAH | ccttttgcctgtgacatttgtgggaggaaatttgccgccacctgttgtctggcccaccat |
| | | HTKIHTGSQKPFQCRICMR | accaagatacacacgggatctcagaagcccttccagtgtcgaatctgcatgcgtaac |
| | | NFSDQSNLRAHIRTHTGEK | ttcagtgaccagtccaacctgcgcgcccacatccgcacccacaccggcgagaagc |
| | | PFACDICGRKFARSAHLSR | cttttgcctgtgacatttgtgggaggaaatttgcccgctccgcccacctgtcccgccat |
| | | HTKIHLRQKDAARGS (SEQ | accaagatacacctgcgccaaaaagatgcggcccggggatcc (SEQ ID |
| | | ID NO: 15) | NO: 69) |
| AUNIP_ | AUNIP_ | GVPAAMAERPFQCRICMR | ggggtacccgccgctatggctgagaggcccttccagtgtcgaatctgcatgcgtaac |
| 14 | B | NFSRSDDLTRHIRTHTGEK | ttcagtcgctccgacgacctgacccgccacatccgcacccacaccggcgagaagc |
| | | PFACDICGRKFAMSYQLGR | cttttgcctgtgacatttgtgggaggaaatttgccatgtcctaccagctgggccgccat |
| | | HTKIHTGSQKPFQCRICMR | accaagatacacacgggcagccaaaagcccttccagtgtcgaatctgcatgcgtaa |
| | | NFSDRSDLSRHIRTHTGEK | cttcagtgaccgctccgacctgtcccgccacatccgcacccacaccggcgagaagc |
| | | PFACDICGRKFARRTDLRR | cttttgcctgtgacatttgtgggaggaaatttgcccgccgcaccgacctgcgccgcca |
| | | HTKIHTGSQKPFQCRICMR | taccaagatacacacgggatctcagaagcccttccagtgtcgaatctgcatgcgtaa |
| | | NFSRSDVLSEHIRTHTGEKP | cttcagtcgctccgacgtgctgtccgagcacatccgcacccacaccggcgagaagc |
| | | FACDICGRKFARSAHLSRH | cttttgcctgtgacatttgtgggaggaaatttgcccgctccgcccacctgtcccgccat |
| | | TKIHLRQKDAARGS (SEQ | accaagatacacctgcgccaaaaagatgcggcccggggatcc (SEQ ID |
| | | ID NO: 16) | NO: 70) |
| AUNIP_ | AUNIP_ | GVPAAMAERPFQCRICMR | ggggtacccgccgctatggctgagaggcccttccagtgtcgaatctgcatgcgtaac |
| 20 | C | NFSQSSNLARHIRTHTGEK | ttcagtcagtcctccaacctggcccgccacatccgcacccacaccggcgagaagcc |
| | | PFACDICGRKFADSSDRKK | ttttgcctgtgacatttgtgggaggaaatttgccgactcctccgaccgcaagaagcata |
| | | HTKIHTGSQKPFQCRICMR | ccaagatacacacgggcagccaaaagcccttccagtgtcgaatctgcatgcgtaact |
| | | NFSHHCCLTAHIRTHTGEK | tcagtcaccactgttgtctgaccgcccacatccgcacccacaccggcgagaagcctt |
| | | PFACDICGRKFARSADLSR | ttgcctgtgacatttgtgggaggaaatttgcccgctccgccgacctgtcccgccatac |
| | | HTKIHTGSQKPFQCRICMR | caagatacacacgggatctcagaagcccttccagtgtcgaatctgcatgcgtaacttc |
| | | NFSDRSHLTRHIRTHTGEK | agtgaccgctcccacctgacccgccacatccgcacccacaccggcgagaagccttt |
| | | PFACDICGRKFAHRSARKR | tgcctgtgacatttgtgggaggaaatttgcccaccgctccgcccgcaagcgccatac |
| | | HTKIHLRQKDAARGS (SEQ | caagatacacctgcgccaaaaagatgcggcccggggatcc (SEQ ID |
| | | ID NO: 17) | NO: 71) |
| AUNIP_ | AUNIP_ | GVPAAMAERPFQCRICMR | ggggtacccgccgctatggctgagaggcccttccagtgtcgaatctgcatgcgtaac |
| 23 | D | NFSRSHSLLRHIRTHTGEKP | ttcagtcgctcccactccctgctgcgccacatccgcacccacaccggcgagaagcc |
| | | FACDICGRKFARLDWLPM | ttttgcctgtgacatttgtgggaggaaatttgcccgcctggactggctgccgatgcata |
| | | HTKIHTGSQKPFQCRICMR | ccaagatacacacgggcagccaaaagcccttccagtgtcgaatctgcatgcgtaact |
| | | NFSTSGSLSRHIRTHTGEKP | tcagtacctccggctccctgtcccgccacatccgcacccacaccggcgagaagcct |
| | | FACDICGRKFARSDDLTRH | tttgcctgtgacatttgtgggaggaaatttgcccgctccgacgacctgacccgccata |
| | | TKIHTGSQKPFQCRICMRN | ccaagatacacacgggatctcagaagcccttccagtgtcgaatctgcatgcgtaactt |
| | | FSERGTLARHIRTHTGEKPF | cagtgagcgcggcaccctggcccgccacatccgcacccacaccggcgagaagcc |
| | | ACDICGRKFARSDDLTRHT | ttttgcctgtgacatttgtgggaggaaatttgcccgctccgacgacctgacccgccata |
| | | KIHLRQKDAARGS (SEQ ID | ccaagatacacctgcgccaaaaagatgcggcccggggatcc (SEQ ID |
| | | NO: 18) | NO: 72) |
| PPP6C_ | PPP6C_ | GVPAAMAERPFQCRICMR | ggggtacccgccgctatggctgagaggcccttccagtgtcgaatctgcatgcgtaac |
| 7 | A | NFSQNAHRKTHIRTHTGEK | ttcagtcagaacgcccaccgcaagacccacatccgcacccacaccggcgagaagc |
| | | PFACDICGRKFARSAHLSR | cttttgcctgtgacatttgtgggaggaaatttgcccgctccgcccacctgtcccgccat |
| | | HTKIHTGSQKPFQCRICMR | accaagatacacacgggcagccaaaagcccttccagtgtcgaatctgcatgcgtaa |
| | | NFSRSDDLTRHIRTHTGEK | cttcagtcgctccgacgacctgacccgccacatccgcacccacaccggcgagaag |
| | | PFACDICGRKFAQSGSLTR | ccttttgcctgtgacatttgtgggaggaaatttgcccagtccggctccctgacccgcca |
| | | HTKIHTGSQKPFQCRICMR | taccaagatacacacgggatctcagaagcccttccagtgtcgaatctgcatgcgtaa |
| | | NFSDRSNLSRHIRTHTGEK | cttcagtgaccgctccaacctgtcccgccacatccgcacccacaccggcgagaagc |
| | | PFACDICGRKFADRTSLRY | cttttgcctgtgacatttgtgggaggaaatttgccgaccgcacctccctgcgctaccat |
| | | HTKIHLRQKDAARGS (SEQ | accaagatacacctgcgccaaaaagatgcggcccggggatcc (SEQ ID |
| | | ID NO: 19) | NO: 73) |
| PPP6C_ | PPP6C_ | GVPAAMAERPFQCRICMR | ggggtacccgccgctatggctgagaggcccttccagtgtcgaatctgcatgcgtaac |
| 12 | B | NFSQSGDLTRHIRTHTGEK | ttcagtcagtccggcgacctgacccgccacatccgcacccacaccggcgagaagc |
| | | PFACDICGRKFARRADLSR | cttttgcctgtgacatttgtgggaggaaatttgcccgccgcgccgacctgtcccgcca |
| | | HTKIHTGSQKPFQCRICMR | taccaagatacacacgggcagccaaaagcccttccagtgtcgaatctgcatgcgtaa |
| | | NFSRSDNLSEHIRTHTGEKP | cttcagtcgctccgacaacctgtccgagcacatccgcacccacaccggcgagaagc |
| | | FACDICGRKFARSADLSRH | cttttgcctgtgacatttgtgggaggaaatttgcccgctccgccgacctgtcccgccat |
| | | TKIHTGSQKPFQCRICMRN | accaagatacacacgggatctcagaagcccttccagtgtcgaatctgcatgcgtaac |
| | | FSQSSDLSRHIRTHTGEKPF | ttcagtcagtcctccgacctgtcccgccacatccgcacccacaccggcgagaagcc |
| | | ACDICGRKFADRSHLARHT | ttttgcctgtgacatttgtgggaggaaatttgccgaccgctcccacctggcccgccata |
| | | KIHLRQKDAARGS (SEQ ID | ccaagatacacctgcgccaaaaagatgcggcccggggatcc (SEQ ID |
| | | NO: 20) | NO: 74) |
| PPP6C_ | PPP6C_ | GVPAAMAERPFQCRICMR | ggggtacccgccgctatggctgagaggcccttccagtgtcgaatctgcatgcgtaac |
| 21 | C | NFSDSSDRKKHIRTHTGEK | ttcagtgactcctccgaccgcaagaagcacatccgcacccacaccggcgagaagc |
| | | PFACDICGRKFARRSDLKR | cttttgcctgtgacatttgtgggaggaaatttgcccgccgctccgacctgaagcgcca |
| | | HTKIHTGSQKPFQCRICMR | taccaagatacacacgggcagccaaaagcccttccagtgtcgaatctgcatgcgtaa |
| | | NFSQSSDLSRHIRTHTGEKP | cttcagtcagtcctccgacctgtcccgccacatccgcacccacaccggcgagaagc |
| | | FACDICGRKFARRDTLLDH | cttttgcctgtgacatttgtgggaggaaatttgcccgccgcgacaccctgctggacca |
| | | TKIHTGSQKPFQCRICMRN | taccaagatacacacgggatctcagaagcccttccagtgtcgaatctgcatgcgtaa |
| | | FSTSGSLSRHIRTHTGEKPF | cttcagtacctccggctccctgtcccgccacatccgcacccacaccggcgagaagc |
| | | ACDICGRKFAQAGQRRVH | cttttgcctgtgacatttgtgggaggaaatttgcccaggccggccagcgccgcgtgc |
| | | TKIHLRQKDAARGS (SEQ | ataccaagatacacctgcgccaaaaagatgcggcccggggatcc (SEQ ID |
| | | ID NO: 21) | NO: 75) |
| PPP6C_ | PPP6C_ | GVPAAMAERPFQCRICMR | ggggtacccgccgctatggctgagaggcccttccagtgtcgaatctgcatgcgtaac |
| 24 | D | NFSQQSDLRVHIRTHTGEK | ttcagtcagcagtccgacctgcgcgtgcacatccgcacccacaccggcgagaagc |
| | | PFACDICGRKFARSADLSR | cttttgcctgtgacatttgtgggaggaaatttgcccgctccgccgacctgtcccgccat |
| | | HTKIHTGSQKPFQCRICMR | accaagatacacacgggcagccaaaagcccttccagtgtcgaatctgcatgcgtaa |
| | | NFSRSDSLLRHIRTHTGEKP | cttcagtcgctccgactccctgctgcgccacatccgcacccacaccggcgagaagc |
| | | FACDICGRKFAQSVRLPHH | cttttgcctgtgacatttgtgggaggaaatttgcccagtccgtgcgcctgccgcaccat |
| | | TKIHTGSQKPFQCRICMRN | accaagatacacacgggatctcagaagcccttccagtgtcgaatctgcatgcgtaac |
| | | FSRKQTRTTHIRTHTGEKPF | ttcagtcgcaagcagacccgcaccacccacatccgcacccacaccggcgagaagc |
| | | ACDICGRKFADSSHRTRHT | cttttgcctgtgacatttgtgggaggaaatttgccgactcctcccaccgcacccgccat |
| | | KIHLRQKDAARGS (SEQ ID | accaagatacacctgcgccaaaaagatgcggcccggggatcc (SEQ ID |
| | | NO: 22) | NO: 76) |
| BAX_6 | BAX_A | GVPAAMAERPFQCRICMR | ggggtacccgccgctatggctgagaggcccttccagtgtcgaatctgcatgcgtaac |
| | | NFSRSDHLSEHIRTHTGEKP | ttcagtcgctccgaccacctgtccgagcacatccgcacccacaccggcgagaagcc |
| | | FACDICGRKFARMYTLSKH | ttttgcctgtgacatttgtgggaggaaatttgcccgcatgtacaccctgtccaagcata |
| | | TKIHTGSQKPFQCRICMRN | ccaagatacacacgggcagccaaaagcccttccagtgtcgaatctgcatgcgtaact |
| | | FSRSDNLSTHIRTHTGEKPF | tcagtcgctccgacaacctgtccacccacatccgcacccacaccggcgagaagcct |
| | | ACDICGRKFARSTHRTQHT | tttgcctgtgacatttgtgggaggaaatttgcccgctccacccaccgcacccagcata |
| | | KIHTGSQKPFQCRICMRNF | ccaagatacacacgggatctcagaagcccttccagtgtcgaatctgcatgcgtaactt |
| | | SRSADLTRHIRTHTGEKPF | cagtcgctccgccgacctgacccgccacatccgcacccacaccggcgagaagcct |
| | | ACDICGRKFAQSGHLSRHT | tttgcctgtgacatttgtgggaggaaatttgcccagtccggccacctgtcccgccatac |
| | | KIHLRQKDAARGS (SEQ ID | caagatacacctgcgccaaaaagatgcggcccggggatcc (SEQ ID |
| | | NO: 35) | NO: 89) |
| BAX_19 | BAX_B | GVPAAMAERPFQCRICMR | ggggtacccgccgctatggctgagaggcccttccagtgtcgaatctgcatgcgtaac |
| | | NFSSEADRSRHIRTHTGEK | ttcagttccgaggccgaccgctcccgccacatccgcacccacaccggcgagaagc |
| | | PFACDICGRKFADRSNLTR | cttttgcctgtgacatttgtgggaggaaatttgccgaccgctccaacctgacccgccat |
| | | HTKIHTGSQKPFQCRICMR | accaagatacacacgggcagccaaaagcccttccagtgtcgaatctgcatgcgtaa |
| | | NFSRSDNLSVHIRTHTGEK | cttcagtcgctccgacaacctgtccgtgcacatccgcacccacaccggcgagaagc |
| | | PFACDICGRKFARRFLRNN | cttttgcctgtgacatttgtgggaggaaatttgcccgccgcttcctgcgcaacaaccat |
| | | HTKIHTGSQKPFQCRICMR | accaagatacacacgggatctcagaagcccttccagtgtcgaatctgcatgcgtaac |
| | | NFSYDYGRYTHIRTHTGEK | ttcagttacgactacggccgctacacccacatccgcacccacaccggcgagaagcc |
| | | PFACDICGRKFADRSHLAR | ttttgcctgtgacatttgtgggaggaaatttgccgaccgctcccacctggcccgccata |
| | | HTKIHLRQKDAARGS (SEQ | ccaagatacacctgcgccaaaaagatgcggcccggggatcc (SEQ ID |
| | | ID NO: 36) | NO: 90) |
| BAX_ | BAX_C | GVPAAMAERPFQCRICMR | ggggtacccgccgctatggctgagaggcccttccagtgtcgaatctgcatgcgtaac |
| 23_ZFP | | NFSDRSHLTRHIRTHTGEK | ttcagtgaccgctcccacctgacccgccacatccgcacccacaccggcgagaagc |
| | | PFACDICGRKFAQSAHLKA | cttttgcctgtgacatttgtgggaggaaatttgcccagtccgcccacctgaaggcccat |
| | | HTKIHTGSQKPFQCRICMR | accaagatacacacgggcagccaaaagcccttccagtgtcgaatctgcatgcgtaa |
| | | NFSRSHSLLRHIRTHTGEKP | cttcagtcgctcccactccctgctgcgccacatccgcacccacaccggcgagaagc |
| | | FACDICGRKFARRDTLLDH | cttttgcctgtgacatttgtgggaggaaatttgcccgccgcgacaccctgctggacca |
| | | TKIHTGSQKPFQCRICMRN | taccaagatacacacgggatctcagaagcccttccagtgtcgaatctgcatgcgtaa |
| | | FSDRSNLSRHIRTHTGEKPF | cttcagtgaccgctccaacctgtcccgccacatccgcacccacaccggcgagaagc |
| | | ACDICGRKFADAFTRTRHT | cttttgcctgtgacatttgtgggaggaaatttgccgacgccttcacccgcacccgccat |
| | | KIHLRQKDAARGS (SEQ ID | accaagatacacctgcgccaaaaagatgcggcccggggatcc (SEQ ID |
| | | NO: 37) | NO: 91) |
| BAX_2 | BAX_D | GVPAAMAERPFQCRICMR | ggggtacccgccgctatggctgagaggcccttccagtgtcgaatctgcatgcgtaac |
| | | NFSRSDAMSQHIRTHTGEK | ttcagtcgctccgacgccatgtcccagcacatccgcacccacaccggcgagaagcc |
| | | PFACDICGRKFARNASRTR | ttttgcctgtgacatttgtgggaggaaatttgcccgcaacgcctcccgcacccgccat |
| | | HTKIHTGSQKPFQCRICMR | accaagatacacacgggcagccaaaagcccttccagtgtcgaatctgcatgcgtaa |
| | | NFSRSADLTRHIRTHTGEK | cttcagtcgctccgccgacctgacccgccacatccgcacccacaccggcgagaag |
| | | PFACDICGRKFARSDDLTR | ccttttgcctgtgacatttgtgggaggaaatttgcccgctccgacgacctgacccgcc |
| | | HTKIHTGSQKPFQCRICMR | ataccaagatacacacgggatctcagaagcccttccagtgtcgaatctgcatgcgta |
| | | NFSHACCRNMHIRTHTGE | acttcagtcacgcctgttgtcgcaacatgcacatccgcacccacaccggcgagaag |
| | | KPFACDICGRKFARSADLS | ccttttgcctgtgacatttgtgggaggaaatttgcccgctccgccgacctgtcccgcca |
| | | RHTKIHLRQKDAARGS | taccaagatacacctgcgccaaaaagatgcggcccggggatcc (SEQ ID |
| | | (SEQ ID NO: 38) | NO: 92) |

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the CD81_8 ZFP amino acid sequence presented in Table 1 (SEQ ID NO: 11). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the CD81_8 ZFP amino acid sequence presented in SEQ ID NO: 11. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the CD81_8 ZFP amino acid sequence presented in SEQ ID NO: 11. In some cases, a zinc finger DNA binding polypeptide includes an amino acid sequence having 90% or more sequence identity (e.g., 95% or more, 97% or more, 98% or more, 99% or more, or 100% sequence identity) with the CD81_8 ZFP amino acid sequence presented in SEQ ID NO: 11. In some cases, a zinc finger DNA binding polypeptide includes an amino acid sequence having the CD81_8 ZFP amino acid sequence presented in SEQ ID NO: 11. In certain embodiments, a DNA sequence encoding a zinc finger DNA binding polypeptide of the disclosure has a nucleotide sequence of the CD81_8 ZFP DNA sequence presented in SEQ ID NO: 65.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the CD81_18 ZFP amino acid sequence presented in Table 1 (SEQ ID NO: 12). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the CD81_18 ZFP amino acid sequence presented in SEQ ID NO: 12. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the CD81_18 ZFP amino acid sequence presented in SEQ ID NO: 12. In some cases, a zinc finger DNA binding polypeptide includes an amino acid sequence having 90% or more sequence identity (e.g., 95% or more, 97% or more, 98% or more, 99% or more, or 100% sequence identity) with the CD81_18 ZFP amino acid sequence presented in SEQ ID NO: 12. In some cases, a zinc finger DNA binding polypeptide includes an amino acid sequence having the CD81_18 ZFP amino acid sequence presented in SEQ ID NO: 12. In certain embodiments, a DNA sequence encoding a zinc finger DNA binding polypeptide of the disclosure has a nucleotide sequence of the CD81_18 ZFP DNA sequence presented in SEQ ID NO: 66.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the CD81_19 ZFP amino acid sequence presented in Table 1 (SEQ ID NO: 13). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the CD81_19 ZFP amino acid sequence presented in SEQ ID NO: 13. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the CD81_19 ZFP amino acid sequence presented in SEQ ID NO: 13. In some cases, a zinc finger DNA binding polypeptide includes an amino acid sequence having 90% or more sequence identity (e.g., 95% or more, 97% or more, 98% or more, 99% or more, or 100% sequence identity) with the CD81_19 ZFP amino acid sequence presented in SEQ ID NO: 13. In some cases, a zinc finger DNA binding polypeptide includes an amino acid sequence having the CD81_19 ZFP amino acid sequence presented in SEQ ID NO: 13. In certain embodiments, a DNA sequence encoding a zinc finger DNA binding polypeptide of the disclosure has a nucleotide sequence of the CD81_19 ZFP DNA sequence presented in SEQ ID NO: 67.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the CD81_2 ZFP amino acid sequence presented in Table 1 (SEQ ID NO: 14). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the CD81_2 ZFP amino acid sequence presented in SEQ ID NO: 14. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the CD81_2 ZFP amino acid sequence presented in SEQ ID NO: 14. In some cases, a zinc finger DNA binding polypeptide includes an amino acid sequence having 90% or more sequence identity (e.g., 95% or more, 97% or more, 98% or more, 99% or more, or 100% sequence identity) with the CD81_2 ZFP amino acid sequence presented in SEQ ID NO: 14. In some cases, a zinc finger DNA binding polypeptide includes an amino acid sequence having the CD81_2 ZFP amino acid sequence presented in SEQ ID NO: 14. In certain embodiments, a DNA sequence encoding a zinc finger DNA binding polypeptide of the disclosure has a nucleotide sequence of the CD81_2 ZFP DNA sequence presented in SEQ ID NO: 68.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the AUNIP_5 ZFP amino acid sequence presented in Table 1 (SEQ ID NO: 15). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the AUNIP_5 ZFP amino acid sequence presented in SEQ ID NO: 15. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the AUNIP_5 ZFP amino acid sequence presented in SEQ ID NO: 15. In some cases, a zinc finger DNA binding polypeptide includes an amino acid sequence having 90% or more sequence identity (e.g., 95% or more, 97% or more, 98% or more, 99% or more, or 100% sequence identity) with the AUNIP_5 ZFP amino acid sequence presented in SEQ ID NO: 15. In some cases, a zinc finger DNA binding polypeptide includes an amino acid sequence having the AUNIP_5 ZFP amino acid sequence presented in SEQ ID NO: 15. In certain embodiments, a DNA sequence encoding a zinc finger DNA binding polypeptide of the disclosure has a nucleotide sequence of the AUNIP_5 ZFP DNA sequence presented in SEQ ID NO: 69.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the AUNIP_14 ZFP amino acid sequence presented in Table 1 (SEQ ID NO: 16). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the AUNIP_14 ZFP amino acid sequence presented in SEQ ID NO: 16. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the AUNIP_14 ZFP amino acid sequence presented in SEQ ID NO: 16. In some cases, a zinc finger DNA binding polypeptide includes an amino acid sequence having 90% or more sequence identity (e.g., 95% or more, 97% or more, 98% or more, 99% or more, or 100% sequence identity) with the AUNIP_14 ZFP amino acid sequence presented in SEQ ID NO: 16. In some cases, a zinc finger DNA binding polypeptide includes an amino acid sequence having the AUNIP_14 ZFP amino acid sequence presented in SEQ ID NO: 16. In certain embodiments, a DNA sequence encoding a zinc finger DNA binding polypeptide of the disclosure has a nucleotide sequence of the AUNIP_14 ZFP DNA sequence presented in SEQ ID NO: 70.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the AUNIP_20 ZFP amino acid sequence presented in Table 1 (SEQ ID NO: 17). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the AUNIP_20 ZFP amino acid sequence presented in SEQ ID NO: 17. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the AUNIP_20 ZFP amino acid sequence presented in SEQ ID NO: 17. In some cases, a zinc finger DNA binding polypeptide includes an amino acid sequence having 90% or more sequence identity (e.g., 95% or more, 97% or more, 98% or more, 99% or more, or 100% sequence identity) with the AUNIP_20 ZFP amino acid sequence presented in SEQ ID NO: 17. In some cases, a zinc finger DNA binding polypeptide includes an amino acid sequence having the AUNIP_20 ZFP amino acid sequence presented in SEQ ID NO: 17. In certain embodiments, a DNA sequence encoding a zinc finger DNA binding polypeptide of the disclosure has a nucleotide sequence of the AUNIP_20 ZFP DNA sequence presented in SEQ ID NO: 71.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the AUNIP_23 ZFP amino acid sequence presented in Table 1 (SEQ ID NO: 18). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the AUNIP_23 ZFP amino acid sequence presented in SEQ ID NO: 18. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the AUNIP_23 ZFP amino acid sequence presented in SEQ ID NO: 18. In some cases, a zinc finger DNA binding polypeptide includes an amino acid sequence having 90% or more sequence identity (e.g., 95% or more, 97% or more, 98% or more, 99% or more, or 100% sequence identity) with the AUNIP_23 ZFP amino acid sequence presented in SEQ ID NO: 18. In some cases, a zinc finger DNA binding polypeptide includes an amino acid sequence having the AUNIP_23 ZFP amino acid sequence presented in SEQ ID NO: 18. In certain embodiments, a DNA sequence encoding a zinc finger DNA binding polypeptide of the disclosure has a nucleotide sequence of the AUNIP_23 ZFP DNA sequence presented in SEQ ID NO: 72.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the PPP6C_7 ZFP amino acid sequence presented in Table 1 (SEQ ID NO: 19). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the PPP6C_7 ZFP amino acid sequence presented in SEQ ID NO: 19. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the PPP6C_7 ZFP amino acid sequence presented in SEQ ID NO: 19. In some cases, a zinc finger DNA binding polypeptide includes an amino acid sequence having 90% or more sequence identity (e.g., 95% or more, 97% or more, 98% or more, 99% or more, or 100% sequence identity) with the PPP6C_7 ZFP amino acid sequence presented in SEQ ID NO: 19. In some cases, a zinc finger DNA binding polypeptide includes an amino acid sequence having the PPP6C_7 ZFP amino acid sequence presented in SEQ ID NO: 19. In certain embodiments, a DNA sequence encoding a zinc finger DNA binding polypeptide of the disclosure has a nucleotide sequence of the PPP6C_7 ZFP DNA sequence presented in SEQ ID NO: 73.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the PPP6C_12 ZFP amino acid sequence presented in Table 1 (SEQ ID NO: 20). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the PPP6C_12 ZFP amino acid sequence presented in SEQ ID NO: 20. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the PPP6C_12 ZFP amino acid sequence presented in SEQ ID NO: 20. In some cases, a zinc finger DNA binding polypeptide includes an amino acid sequence having 90% or more sequence identity (e.g., 95% or more, 97% or more, 98% or more, 99% or more, or 100% sequence identity) with the PPP6C_12 ZFP amino acid sequence presented in SEQ ID NO: 20. In some cases, a zinc finger DNA binding polypeptide includes an amino acid sequence having the PPP6C_12 ZFP amino acid sequence presented in SEQ ID NO: 20. In certain embodiments, a DNA sequence encoding a zinc finger DNA binding polypeptide of the disclosure has a nucleotide sequence of the PPP6C_12 ZFP DNA sequence presented in SEQ ID NO: 74.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the PPP6C_21 ZFP amino acid sequence presented in Table 1 (SEQ ID NO: 21). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the PPP6C_21 ZFP amino acid sequence presented in SEQ ID NO: 21. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the PPP6C_21 ZFP amino acid sequence presented in SEQ ID NO: 21. In some cases, a zinc finger DNA binding polypeptide includes an amino acid sequence having 90% or more sequence identity (e.g., 95% or more, 97% or more, 98% or more, 99% or more, or 100% sequence identity) with the PPP6C_21 ZFP amino acid sequence presented in SEQ ID NO: 21. In some cases, a zinc finger DNA binding polypeptide includes an amino acid sequence having the PPP6C_21 ZFP amino acid sequence presented in SEQ ID NO: 21. In certain embodiments, a DNA sequence encoding a zinc finger DNA binding polypeptide of the disclosure has a nucleotide sequence of the PPP6C_21 ZFP DNA sequence presented in SEQ ID NO: 75.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the PPP6C_24 ZFP amino acid sequence presented in Table 1 (SEQ ID NO: 22). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the PPP6C_24 ZFP amino acid sequence presented in SEQ ID NO: 22. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the PPP6C_24 ZFP amino acid sequence presented in SEQ ID NO: 22. In some cases, a zinc finger DNA binding polypeptide includes an amino acid sequence having 90% or more sequence identity (e.g., 95% or more, 97% or more, 98% or more, 99% or more, or 100% sequence identity) with the PPP6C_24 ZFP amino acid sequence presented in SEQ ID NO: 22. In some cases, a zinc finger DNA binding polypeptide includes an amino acid sequence having the PPP6C_24 ZFP amino acid sequence presented in SEQ ID NO: 22. In certain embodiments, a DNA sequence encoding a zinc finger DNA binding polypeptide of the disclosure has a nucleotide sequence of the PPP6C_24 ZFP DNA sequence presented in SEQ ID NO: 76.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the BAX_6 ZFP amino acid sequence presented in Table 1 (SEQ ID NO: 35). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the BAX_6 ZFP amino acid sequence presented in SEQ ID NO: 35. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the BAX_6 ZFP amino acid sequence presented in SEQ ID NO: 35. In some cases, a zinc finger DNA binding polypeptide includes an amino acid sequence having 90% or more sequence identity (e.g., 95% or more, 97% or more, 98% or more, 99% or more, or 100% sequence identity) with the BAX_6 ZFP amino acid sequence presented in SEQ ID NO: 35. In some cases, a zinc finger DNA binding polypeptide includes an amino acid sequence having the BAX_6 ZFP amino acid sequence presented in SEQ ID NO: 35. In certain embodiments, a DNA sequence encoding a zinc finger DNA binding polypeptide of the disclosure has a nucleotide sequence of the BAX_6 ZFP DNA sequence presented in SEQ ID NO: 89.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the BAX_19 ZFP amino acid sequence presented in Table 1 (SEQ ID NO: 36). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the BAX_19 ZFP amino acid sequence presented in SEQ ID NO: 36. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the BAX_19 ZFP amino acid sequence presented in SEQ ID NO: 36. In some cases, a zinc finger DNA binding polypeptide includes an amino acid sequence having 90% or more sequence identity (e.g., 95% or more, 97% or more, 98% or more, 99% or more, or 100% sequence identity) with the BAX_19 ZFP amino acid sequence presented in SEQ ID NO: 36. In some cases, a zinc finger DNA binding polypeptide includes an amino acid sequence having the BAX_19 ZFP amino acid sequence presented in SEQ ID NO: 36. In certain embodiments, a DNA sequence encoding a zinc finger DNA binding polypeptide of the disclosure has a nucleotide sequence of the BAX_19 ZFP DNA sequence presented in SEQ ID NO: 90.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the BAX_23_ZFP ZFP amino acid sequence presented in Table 1 (SEQ ID NO: 37). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the BAX_23_ZFP ZFP amino acid sequence presented in SEQ ID NO: 37. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the BAX_23_ZFP ZFP amino acid sequence presented in SEQ ID NO: 37. In some cases, a zinc finger DNA binding polypeptide includes an amino acid sequence having 90% or more sequence identity (e.g., 95% or more, 97% or more, 98% or more, 99% or more, or 100% sequence identity) with the BAX_23_ZFP ZFP amino acid sequence presented in SEQ ID NO: 37. In some cases, a zinc finger DNA binding polypeptide includes an amino acid sequence having the BAX_23_ZFP ZFP amino acid sequence presented in SEQ ID NO: 37. In certain embodiments, a DNA sequence encoding a zinc finger DNA binding polypeptide of the disclosure has a nucleotide sequence of the BAX_23_ZFP ZFP DNA sequence presented in SEQ ID NO: 91.

In some cases, a zinc finger DNA binding polypeptide of the disclosure includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the BAX_2 ZFP amino acid sequence presented in Table 1 (SEQ ID NO: 38). For example, in some cases, a zinc finger DNA binding polypeptide includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the BAX_2 ZFP amino acid sequence presented in SEQ ID NO: 38. In some cases, a zinc finger DNA binding polypeptide includes an amino acid sequence having 80% or more sequence identity (e.g., 85% or more, 90% or more, 95% or more, 97% or more, 98% or more, 99% or more, or 100% sequence identity) with the BAX_2 ZFP amino acid sequence presented in SEQ ID NO: 38. In some cases, a zinc finger DNA binding polypeptide includes an amino acid sequence having 90% or more sequence identity (e.g., 95% or more, 97% or more, 98% or more, 99% or more, or 100% sequence identity) with the BAX_2 ZFP amino acid sequence presented in SEQ ID NO: 38. In some cases, a zinc finger DNA binding polypeptide includes an amino acid sequence having the BAX_2 ZFP amino acid sequence presented in SEQ ID NO: 38. In certain embodiments, a DNA sequence encoding a zinc finger DNA binding polypeptide of the disclosure has a nucleotide sequence of the BAX_2 ZFP DNA sequence presented in SEQ ID NO: 92.

### Epigenome Modifying Polypeptides

As discussed above, aspects of the subject fusion proteins include epigenome modifying polypeptides. Any polypeptide configured to modify a target nucleic acid or a protein produced therefrom at the level of the epigenome may be employed. In certain cases, the epigenome modifying polypeptide comprises a DNA methyltransferase domain. The term “DNA methyltransferase” as provided herein refers to an enzyme that catalyzes the transfer of a methyl group to DNA. Non-limiting examples of DNA methyltransferases include Dnmt1, Dnmt3A, Dnmt3B, and Dnmt3L. In aspects, the DNA methyltransferase is a bacterial cytosine methyltransferase and/or a bacterial non-cytosine methyltransferase. Depending on the specific DNA methyltransferase, different regions of DNA are methylated. For example, Dnmt3A typically targets CpG dinucleotides for methylation. Through DNA methylation, DNA methyltransferases can modify the activity of a DNA segment (e.g., gene expression) without altering the DNA sequence. In aspects, DNA methylation results in repression of gene transcription and/or modulation of methylation sensitive transcription factors or CTCF. As described herein, fusion proteins may include one or more (e.g., two) DNA methyltransferases. When a DNA methyltransferase is included as part of a fusion protein, the DNA methyltransferase may be referred to as a “DNA methyltransferase domain.” In aspects, a DNA methyltransferase domain includes one or more DNA methyltransferases. In aspects, a DNA methyltransferase domain includes two DNA methyltransferases.

In aspects, the DNA methyltransferase domain is and/or comprises Dnmt3A. In aspects, the DNA methyltransferase domain has the amino acid sequence of NHDQEFDPPKVYPPVPAEKRKPIRVLSLFDGIATGLLVLKDLGIQVDRYIASEVCEDSITVGMVR HQGKIMYVGDVRSVTQKHIQEWGPFDLVIGGSPCNDLSIVNPARKGLYEGTGRLFFEFYRLLHD ARPKEGDDRPFFWLFENVVAMGVSDKRDISRFLESNPVMIDAKEVSAAHRARYFWGNLPGMN RPLASTVNDKLELQECLEHGRIAKFSKVRTITTRSNSIKQGKDQHFPVFMNEKEDILWCTEMERV FGFPVHYTDVSNMSRLARQRLLGRSWSVPVIRHLFAPLKEYFACV (SEQ ID NO: 93). In aspects, the DNA methyltransferase domain has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to NHDQEFDPPKVYPPVPAEKRKPIRVLSLFDGIATGLLVLKDLGIQVDRYIASEVCEDSITVGMVR HQGKIMYVGDVRSVTQKHIQEWGPFDLVIGGSPCNDLSIVNPARKGLYEGTGRLFFEFYRLLHD ARPKEGDDRPFFWLFENVVAMGVSDKRDISRFLESNPVMIDAKEVSAAHRARYFWGNLPGMN RPLASTVNDKLELQECLEHGRIAKFSKVRTITTRSNSIKQGKDQHFPVFMNEKEDILWCTEMERV FGFPVHYTDVSNMSRLARQRLLGRSWSVPVIRHLFAPLKEYFACV (SEQ ID NO: 93). In aspects, the DNA methyltransferase domain has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to

(SEQ ID NO: 94)
MNHDQEFDPPKVYPPVPAEKRKPIRVLSLFDGIATGLLVLKDLGIQ
VDRYIASEVCEDSITVGMVRHQGKIMYVGDVRSVTQKHIQEWGPFD
LVIGGSPCNDLSIVNPARKGLYEGTGRLFFEFYRLLHDARPKEGDD
RPFFWLFENVVAMGVSDKRDISRFLESNPVMIDAKEVSAAHRARYF
WGNLPGMNRPLASTVNDKLELQECLEHGRIAKFSKVRTITTRSNSI
KQGKDQHFPVFMNEKEDILWCTEMERVFGFPVHYTDVSNMSRLARQ
RLLGRSWSVPVIRHLFAPLKEYFACV.

A “Dnmt3A”, “Dnmt3a,” “DNA (cytosine-5)-methyltransferase 3A” or “DNA methyltransferase 3a” protein as referred to herein includes any of the recombinant or naturally-occurring forms of the Dnmt3A enzyme or variants or homologs thereof that maintain Dnmt3A enzyme activity (e.g. within at least 50%, 55%, 60%, 65%, 70%, 75%, 80%, 85%, 90%, 91%, 92%, 93%, 94%, 95%, 96%, 97%, 98%, 99% or 100% activity compared to Dnmt3A). In aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Dnmt3A protein. In aspects, the Dnmt3A protein is substantially identical to the protein identified by the UniProt reference number Q9Y6K1 or a variant or homolog having substantial identity thereto. In aspects, the Dnmt3A polypeptide is encoded by a nucleic acid sequence identified by the NCBI reference sequence Accession number NM_022552, homologs or functional fragments thereof.

In aspects, the DNA methyltransferase domain is and/or comprises Dnmt3L. In aspects, the DNA methyltransferase domain has the amino acid sequence of MGPMEIYKTVSAWKRQPVRVLSLFRNIDKVLKSLGFLESGSGSGGGTLKYVEDVTNVVRRDVE KWGPFDLVYGSTQPLGSSCDRCPGWYMFQFHRILQYALPRQESQRPFFWIFMDNLLLTEDDQET TTRFLQTEAVTLQDVRGRDYQNAMRVWSNIPGLKSKHAPLTPKEEEYLQAQVRSRSKLDAPKV DLLVKNCLLPLREYFKYFSQNSLPL (SEQ ID NO: 95). In aspects, the DNA methyltransferase domain has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to

(SEQ ID NO: 95)
MGPMEIYKTVSAWKRQPVRVLSLFRNIDKVLKSLGFLESGSGSGGG
TLKYVEDVTNVVRRDVEKWGPFDLVYGSTQPLGSSCDRCPGWYMFQ
FHRILQYALPRQESQRPFFWIFMDNLLLTEDDQETTTRFLQTEAVT
LQDVRGRDYQNAMRVWSNIPGLKSKHAPLTPKEEEYLQAQVRSRSK
LDAPKVDLLVKNCLLPLREYFKYFSQNSLPL.

A “Dnmt3L”, “DNA (cytosine-5)-methyltransferase 3L” or “DNA methyltransferase 3L” protein as referred to herein includes any of the recombinant or naturally-occurring forms of the Dnmt3L enzyme or variants or homologs thereof that maintain Dnmt3L enzyme activity (e.g., within at least 50%, 55%, 60%, 65%, 70%, 75%, 80%, 85%, 90%, 91%, 92%, 93%, 94%, 95%, 96%, 97%, 98%, 99% or 100% activity compared to Dnmt3L). A “Dnmt3L”, “DNA (cytosine-5)-methyltransferase 3L” or “DNA methyltransferase 3L” protein as referred to herein includes any of the recombinant or naturally-occurring forms of the Dnmt3L enzyme or variants or homologs thereof that maintain Dnmt3L enzyme activity (e.g., within at least 50%, 55%, 60%, 65%, 70%, 75%, 80%, 85%, 90%, 91%, 92%, 93%, 94%, 95%, 96%, 97%, 98%, 99% or 100% activity compared to Dnmt3L). In aspects, the variants or homologs have at least 50%, 55%, 60%, 65%, 70%, 75%, 80%, 85%, 90%, 91%, 92%, 93%, 94%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Dnmt3L protein. In aspects, the Dnmt3L protein is substantially identical to the protein identified by the UniProt reference number Q9CWR8 or a variant or homolog having substantial identity thereto. For example, a suitable protein identified by the UniProt reference number Q9CWR8 may include the mouse sequence MGSRETPSSCSKTLETLDLETSDSSSPDADSPLEEQWLKSSPALKEDSVDVVLEDCKEPLSPSSPP TGREMIRYEVKVNRRSIEDICLCCGTLQVYTRHPLFEGGLCAPCKDKFLESLFLYDDDGHQSYC TICCSGGTLFICESPDCTRCYCFECVDILVGPGTSERINAMACWVCFLCLPFSRSGLLQRRKRWR HQLKAFHDQEGAGPMEIYKTVSAWKRQPVRVLSLFRNIDKVLKSLGFLESGSGSGGGTLKYVE DVTNVVRRDVEKWGPFDLVYGSTQPLGSSCDRCPGWYMFQFHRILQYALPRQESQRPFFWIFM DNLLLTEDDQETTTRFLQTEAVTLQDVRGRDYQNAMRVWSNIPGLKSKHAPLTPKEEEYLQAQ VRSRSKLDAPKVDLLVKNCLLPLREYFKYFSQNSLPL (SEQ ID NO: 96). In aspects, the Dnmt3L protein is identical to the protein identified by the UniProt reference number Q9CWR8. In aspects, the Dnmt3L protein has at least 75% sequence identity to the amino acid sequence of the protein identified by the UniProt reference number Q9CWR8. In aspects, the Dnmt3L protein has at least 80% sequence identity to the amino acid sequence of the protein identified by the UniProt reference number Q9CWR8. In aspects, the Dnmt3L protein has at least 85% sequence identity to the amino acid sequence of the protein identified by the UniProt reference number Q9CWR8. In aspects, the Dnmt3L protein has at least 95% sequence identity to the amino acid sequence of the protein identified by the UniProt reference number Q9CWR8. In aspects, the Dnmt3L polypeptide is encoded by the following mouse-derived nucleic acid sequence identified by the NCBI reference sequence Accession number NM_001081695:

(SEQ ID NO: 97)
aacagaaaggaaatcaaaaccacctgcctgcctcaccgcccagaaa
ctcagcctttgggacagtaagacgctgagaggctgtgggcctcctc
caatgctcagatgctgagaaggaagcattaggtgatgccaaaatgg
gcttcctgagaaacaccacctcttcctcatccccaaaggaggtttc
tgctcactggcctctcccactgtctcaggcagggttctgacgaccc
tgctgtcacacccgccatcccttggacgcagacccttctagccgat
tacatcaatgggttcccgggagacaccttcttcttgctctaagacc
cttgaaaccttggacctggagacttccgacagctctagccctgatg
ctgacagtcctctggaagagcaatggctgaaatcctccccagccct
gaaggaggacagtgtggatgtggtactggaagactgcaaagagcct
ctgtccccctcctcgcctccgacaggcagagagatgatcaggtacg
aagtcaaagtgaaccgacggagcattgaagacatctgcctctgctg
tggaactctccaggtgtacactcggcaccccttgtttgagggaggg
ttatgtgccccatgtaaggataagttcctggagtccctcttcctgt
atgatgatgatggacaccagagttactgcaccatctgctgttccgg
gggtaccctgttcatctgtgagagccccgactgtaccagatgctac
tgtttcgagtgtgtggacatcctggtgggccccgggacctcagaga
ggatcaatgccatggcctgctgggtttgcttcctgtgcctgccctt
ctcacggagtggactgctgcagaggcgcaagaggtggcggcaccag
ctgaaggccttccatgatcaagagggagcgggccctatggagatat
acaagacagtgtctgcatggaagagacagccagtgcgggtactgag
cctttttagaaatattgataaagtactaaagagtttgggctttttg
gaaagcggttctggttctgggggaggaacgctgaagtacgtggaag
atgtcacaaatgtcgtgaggagagacgtggagaaatggggcccctt
tgacctggtgtacggctcgacgcagcccctaggcagctcttgtgat
cgctgtcccggctggtacatgttccagttccaccggatcctgcagt
atgcgctgcctcgccaggagagtcagcggcccttcttctggatatt
catggacaatctgctgctgactgaggatgaccaagagacaactacc
cgcttccttcagacagaggctgtgaccctccaggatgtccgtggca
gagactaccagaatgctatgcgggtgtggagcaacattccagggct
gaagagcaagcatgcgcccctgaccccaaaggaagaagagtatctg
caagcccaagtcagaagcaggagcaagctggacgccccgaaagttg
acctcctggtgaagaactgccttctcccgctgagagagtacttcaa
gtatttttctcaaaactcacttcctctttagaaatgaatcaccata
agatgaaagtctttcctagaaccagggcagatttcttcctaaggtc
tcttccctccacagttttctctggtttgctttcaggccttcgggtt
tctctcctgtttgattgccaggatgcctctgtgcagctcactttgc
ggggtgggaggtgcctacggctctgcacaagttcccggtgggataa
cctgccatgtttctctgaaactgtgtgtacctgttgtgaagttttt
caaatatatcataggattgttactggtaaaaaaaaaa.

In aspects, the DNA methyltransferase domain includes Dnmt3A and Dnmt3L. In such aspects, the DNA methyltransferase polypeptide may be referred to as a DNMT3A-3L polypeptide. In aspects, the DNA methyltransferase domain has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to NHDQEFDPPKVYPPVPAEKRKPIRVLSLFDGIATGLLVLKDLGIQVDRYIASEVCEDSITVGMVR HQGKIMYVGDVRSVTQKHIQEWGPFDLVIGGSPCNDLSIVNPARKGLYEGTGRLFFEFYRLLHD ARPKEGDDRPFFWLFENVVAMGVSDKRDISRFLESNPVMIDAKEVSAAHRARYFWGNLPGMN RPLASTVNDKLELQECLEHGRIAKFSKVRTITTRSNSIKQGKDQHFPVFMNEKEDILWCTEMERV FGFPVHYTDVSNMSRLARQRLLGRSWSVPVIRHLFAPLKEYFACVSSGNSNANSRGPSFSSGLVP LSLRGSHMGPMEIYKTVSAWKRQPVRVLSLFRNIDKVLKSLGFLESGSGSGGGTLKYVEDVTN VVRRDVEKWGPFDLVYGSTQPLGSSCDRCPGWYMFQFHRILQYALPRQESQRPFFWIFMDNLL LTEDDQETTTRFLQTEAVTLQDVRGRDYQNAMRVWSNIPGLKSKHAPLTPKEEEYLQAQVRSR SKLDAPKVDLLVKNCLLPLREYFKYFSQNSLPL (SEQ ID NO: 98). A description of Dnmt3A-3L domain structure and use may be found, for example, in Siddique et al, J. Mol. Biol. 425, 2013 and Stepper et al, Nucleic Acids Res. 45, 2017, which are incorporated herein by reference in their entirety and for all purposes. In aspects, the Dnmt3A and the Dnmt3L are covalently linked. In aspects, the Dnmt3A is covalently linked to the Dnmt3L through a peptide linker. In aspects, the peptide linker includes the sequence set forth by SSGNSNANSRGPSFSSGLVPLSLRGSH (SEQ ID NO: 99). In aspects, the peptide linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to

(SEQ ID NO: 99)
SSGNSNANSRGPSFSSGLVPLSLRGSH.

### Regulatory Domains

The fusion proteins described herein can optionally be associated with regulatory domains for modulation of gene expression. The ZFP can be covalently or non-covalently associated with one or more regulatory domains, alternatively two or more regulatory domains, with the two or more domains being two copies of the same domain, or two different domains. The regulatory domains can be covalently linked to the ZFP, e.g., via an amino acid linker, as part of the fusion protein. The ZFPs can also be associated with a regulatory domain via a non-covalent dimerization domain, e.g., a leucine zipper, a STAT protein N terminal domain, or an FK506 binding protein (see, e.g., O'Shea, Science 254: 539 (1991), Barahmand-Pour et al, Curr. Top. Microbiol. Immunol. 211:121-128 (1996); Klemm et al., Annu. Rev. Immunol. 16:569-592 (1998); Klemm et al., Annu. Rev. Immunol. 16:569-592 (1998); Ho et al., Nature 382:822-826 (1996); and Pomeranz et al., Biochem. 37:965 (1998)). The regulatory domain can be associated with the ZFP at any suitable position, including the C- or N-terminus of the fusion protein.

Common regulatory domains for addition to the ZFP include, e.g., effector domains from transcription factors (activators, repressors, co-activators, co-repressors), silencers, nuclear hormone receptors, oncogene transcription factors (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g., kinases, acetylases and deacetylases); and DNA modifying enzymes (e.g., methyltransferases, topoisomerases, helicases, ligases, kinases, phosphatases, polymerases, endonucleases) and their associated factors and modifiers.

Transcription factor polypeptides from which one can obtain a regulatory domain include those that are involved in regulated and basal transcription. Such polypeptides include transcription factors, their effector domains, coactivators, silencers, nuclear hormone receptors (see, e.g., Goodrich et al., Cell 84:825-30 (1996) for a review of proteins and nucleic acid elements involved in transcription; transcription factors in general are reviewed in Barnes & Adcock, Clin. Exp. Allergy 25 Suppl. 2:46-9 (1995) and Roeder, Methods Enzymol. 273:165-71 (1996)). Databases dedicated to transcription factors are known (see, e.g., Science 269:630 (1995)). Nuclear hormone receptor transcription factors are described in, for example, Rosen et al., J Med. Chem. 38:4855-74 (1995). The C/EBP family of transcription factors are reviewed in Wedel et al., Immunobiology 193:171-85 (1995). Coactivators and co-repressors that mediate transcription regulation by nuclear hormone receptors are reviewed in, for example, Meier, Eur. J Endocrinol. 134 (2):158-9 (1996); Kaiser et al., Trends Biochem. Sci. 21:342-5 (1996); and Utley et al., Nature 394:498-502 (1998)). GATA transcription factors, which are involved in regulation of hematopoiesis, are described in, for example, Simon, Nat. Genet. 11:9-11 (1995); Weiss et al., Exp. Hematol. 23:99-107. TATA box binding protein (TBP) and its associated TAP polypeptides (which include TAF30, TAF55, TAF80, TAF 10, TAF150, and TAF250) are described in Goodrich & Tjian, Curr. Opin. Cell Biol. 6:403-9 (1994) and Hurley, Curr. Opin. Struct. Biol. 6:69-75 (1996). The STAT family of transcription factors are reviewed in, for example, Barahmand-Pour et al., Curr. Top. Microbiol. Immunol. 211:121-8 (1996). Transcription factors involved in disease are reviewed in Aso et al., J Clin. Invest. 97:1561-9 (1996).

Examples of proteins (or fragments thereof) that can be used in increase transcription include but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1, and the like; histone lysine demethylases such as JHDM2a/b, UTX, JMJD3, and the like; histone acetyltransferases such as GCN5, PCAF, CBP, p300, TAF1, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TET1CD), TET1, DME, DML1, DML2, ROS1, and the like.

In some cases, the fusion protein includes a transcriptional repression polypeptide. Examples of proteins (or fragments thereof) that can be used in decrease transcription include but are not limited to: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; and periphery recruitment elements such as Lamin A, Lamin B, and the like.

In certain cases, the transcriptional repression polypeptide is a Krüppel associated box (KRAB) polypeptide. A “KRAB polypeptide” as provided herein refers to a category of transcriptional repression domains present in approximately 400 human zinc finger protein—based transcription factors. KRAB domains typically include about 45 to about 75 amino acid residues. In one embodiment, the KRAB repression domain from the human KOX-1 protein is used as a transcriptional repressor (Thiesen et al., New Biologist 2:363-374 (1990); Margolin et al., PNAS 91:4509-4513 (1994); Pengue et al., Nucl. Acids Res. 22:2908-2914 (1994); Witzgall et al., PNAS 91:4514-4518 (1994)). In another embodiment, KAP-1, a KRAB co-repressor, is used with KRAB (Friedman et al., Genes Dev. 10:2067-2078 (1996)). Alternatively, KAP-1 can be used alone with a ZFP. In aspects, the KRAB domain includes an amino acid sequence that has at least 75% sequence identity to DAKSLTAWSRTLVTFKDVFVDFTREEWKLLDTAQQIVYRNVMLENYKNLVSLGYQLTKPDVIL RLEKGEEP (SEQ ID NO: 100). In aspects, the KRAB domain includes an amino acid sequence that has at least 80% sequence identity, 90% sequence identity, or 95% sequence identity to

(SEQ ID NO: 100)
DAKSLTAWSRTLVTFKDVFVDFTREEWKLLDTAQQIVYRNVMLEN
YKNLVSLGYQLTKPDVILRLEKGEEP.

Other transcription factors and transcription factor domains that act as transcriptional repressors include MAD (see, e.g., Sommer et al., J. Biol. Chem. 273:6632-6642 (1998); Gupta et al., Oncogene 16:1149-1159 (1998); Queva et al., Oncogene 16:967-977 (1998); Larsson et al., Oncogene 15:737-748 (1997); Laherty et al., Cell 89:349-356 (1997); and Cultraro et al, Mol. Cell. Biol. 17:2353-2359 (19977)); FKHR (forkhead in rhapdosarcoma gene; Ginsberg et al., Cancer Res. 15:3542-3546 (1998); Epstein et al, Mol. Cell. Biol. 18:4118-4130 (1998)); EGR-1 (early growth response gene product-1; Yan et al., PNAS 95:8298-8303 (1998); and Liu et al., Cancer Gene Ther. 5:3-28 (1998)); the ets2 repressor factor repressor domain (ERD; Sgouras et al., EMBO J. 14:4781-4793 ((19095)); and the MAD smSIN3 interaction domain (SID; Ayer et al., Mol. Cell. Biol. 16:5772-5781 (1996)). In select versions, the transcriptional repression polypeptide includes a histone deacetylase, such as those described in Jin & Scotto, Mol. Cell. Biol. 18:4377-4384 (1998); Syntichaki & Thireos, J. Biol. Chem. 273:24414-24419 (1998); Sakaguchi et al., Genes Dev. 12:2831-2841 (1998); and Martinez et al, J. Biol. Chem. 273:23781-23785 (1998).

Kinases, phosphatases, and other proteins that modify polypeptides involved in gene regulation are also useful as regulatory domains for ZFPs. Such modifiers are often involved in switching on or off transcription mediated by, for example, hormones. Kinases involved in transcriptional regulation are reviewed in Davis, Mol. Reprod. Dev. 42:459-67 (1995), Jackson et al., Adv. Second Messenger Phosphoprotein Res. 28:279-86 (1993), and Boulikas, Crit. Rev. Eukaryot. Gene Expr. 5:1-77 (1995), while phosphatases are reviewed in, for example, Schonthal & Semin, Cancer Biol. 6:239-48 (1995). Nuclear tyrosine kinases are described in Wang, Trends Biochem. Sci. 19:373-6 (1994).

As described, useful domains can also be obtained from the gene products of oncogenes (e.g., myc, jun, fos, myb, max, mad, rel, ets, bcl, myb, mos family members) and their associated factors and modifiers. Oncogenes are described in, for example, Cooper, Oncogenes, 2nd ed., The Jones and Bartlett Series in Biology, Boston, Mass., Jones and Bartlett Publishers, 1995. The ets transcription factors are reviewed in Waslylk et al., Eur. J. Biochem. 211:7-18 (1993) and Crepieux et al., Crit. Rev. Oncog. 5:615-38 (1994). Myc oncogenes are reviewed in, for example, Ryan et al., Biochem. J. 314:713-21 (1996). The jun and fos transcription factors are described in, for example, The Fos and Jun Families of Transcription Factors, Angel & Herrlich, eds. (1994). The max oncogene is reviewed in Hurlin et al., Cold Spring Harb. Symp. Quant. Biol. 59:109-16. The myb gene family is reviewed in Kanci-Ishii et al., Curr. Top. Microbiol. Immunol. 211:89-98 (1996). The mos family is reviewed in Yew et al., Curr. Opin. Genet. Dev. 3:19-25 (1993).

Fusion proteins can include regulatory domains obtained from DNA repair enzymes and their associated factors and modifiers. DNA repair systems are reviewed in, for example, Vos, Curr. Opin. Cell Biol. 4:385-95 (1992); Sancar, Ann. Rev. Genet. 29:69-105 (1995); Lehmann, Genet. Eng. 17:1-19 (1995); and Wood, Ann. Rev. Biochem. 65:135-67 (1996). DNA rearrangement enzymes and their associated factors and modifiers can also be used as regulatory domains (see, e.g., Gangloff et al., Experientia 50:261-9 (1994); Sadowski, FASEB J. 7:760-7 (1993)).

The fusion protein may include amino acid sequences useful for targeting the fusion protein to specific regions of a cell (e.g., cytoplasm, nucleus). Thus, in aspects, the fusion protein further includes a nuclear localization signal (NLS) peptide. In sone such cases, the fusion protein includes a plurality of (e.g., 2) NLS peptides. Non-limiting examples of NLSs include an NLS sequence derived from: the NLS of the SV40 virus large T-antigen, having the amino acid sequence PKKKRKV (SEQ ID NO: 101); the NLS from nucleoplasmin (e.g., the nucleoplasmin bipartite NLS with the sequence KRPAATKKAGQAKKKK (SEQ ID NO: 102)); the c-myc NLS having the amino acid sequence PAAKRVKLD (SEQ ID NO: 103) or RQRRNELKRSP (SEQ ID NO: 104); the hRNPA1 M9 NLS having the sequence NQSSNFGPMKGGNFGGRSSGPYGGGGQYFAKPRNQGGY (SEQ ID NO: 105); the sequence RMRIZFKNKGKDTAELRRRRVEVSVELRKAKKDEQILKRRNV (SEQ ID NO: 106) of the IBB domain from importin-alpha; the sequences VSRKRPRP (SEQ ID NO: 107) and PPKKARED (SEQ ID NO: 108) of the myoma T protein; the sequence PQPKKKPL (SEQ ID NO: 109) of human p53; the sequence SALIKKKKKMAP (SEQ ID NO: 110) of mouse c-abl IV; the sequences DRLRR (SEQ ID NO: 111) and PKQKKRK (SEQ ID NO: 112) of the influenza virus NS1; the sequence RKLKKKIKKL (SEQ ID NO: 113) of the Hepatitis virus delta antigen; the sequence REKKKFLKRR (SEQ ID NO: 114) of the mouse Mx1 protein; the sequence KRKGDEVDGVDEVAKKKSKK (SEQ ID NO: 115) of the human poly(ADP-ribose) polymerase; and the sequence RKCLQAGMNLEARKTKK (SEQ ID NO: 116) of the steroid hormone receptors (human) glucocorticoid. In general, NLS (or multiple NLSs) are of sufficient strength to drive accumulation of the fusion protein in a detectable amount in the nucleus of a eukaryotic cell. Detection of accumulation in the nucleus may be performed by any suitable technique. For example, a detectable marker may be fused to the fusion protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly. In aspects, the NLS is the sequence set forth by PKKKRKV (SEQ ID NO 101). In aspects, the NLS has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to PKKKRKV (SEQ ID NO: 101).

### Linkers

In some embodiments, a subject fusion protein can include a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

In embodiments, the linker is a peptide linker. A “peptide linker” as provided herein is a linker including a peptide moiety. In embodiments, the peptide linker is a divalent peptide, such as an amino acid sequence attached at the N-terminus and the C-terminus to the remainder of the compound (e.g., fusion protein provided herein). The peptide linker may be a peptide moiety (a divalent peptide moiety) capable of being cleaved (e.g., a P2A cleavable polypeptide). A peptide linker as provided herein may also be referred to interchangeably as an amino acid linker. In aspects, the peptide linker includes 1 to about 80 amino acid residues. In aspects, the peptide linker includes 1 to about 70 amino acid residues. In aspects, the peptide linker includes 1 to about 60 amino acid residues. In aspects, the peptide linker includes 1 to about 50 amino acid residues. In aspects, the peptide linker includes 1 to about 40 amino acid residues. In aspects, the peptide linker includes 1 to about 30 amino acid residues. In aspects, the peptide linker includes 1 to about 25 amino acid residues. In aspects, the peptide linker includes 1 to about 20 amino acid residues. In aspects, the peptide linker includes about 2 to about 20 amino acid residues. In aspects, the peptide linker includes about 2 to about 19 amino acid residues. In aspects, the peptide linker includes about 2 to about 18 amino acid residues. In aspects, the peptide linker includes about 2 to about 17 amino acid residues. In aspects, the peptide linker includes about 2 to about 16 amino acid residues. In aspects, the peptide linker includes about 2 to about 15 amino acid residues. In aspects, the peptide linker includes about 2 to about 14 amino acid residues. In aspects, the peptide linker includes about 2 to about 13 amino acid residues. In aspects, the peptide linker includes about 2 to about 12 amino acid residues. In aspects, the peptide linker includes about 2 to about 11 amino acid residues. In aspects, the peptide linker includes about 2 to about 10 amino acid residues. In aspects, the peptide linker includes about 2 to about 9 amino acid residues. In aspects, the peptide linker includes about 2 to about 8 amino acid residues. In aspects, the peptide linker includes about 2 to about 7 amino acid residues. In aspects, the peptide linker includes about 2 to about 6 amino acid residues. In aspects, the peptide linker includes about 2 to about 5 amino acid residues. In aspects, the peptide linker includes about 2 to about 4 amino acid residues. In aspects, the peptide linker includes about 2 to about 3 amino acid residues. In aspects, the peptide linker includes about 3 to about 19 amino acid residues. In aspects, the peptide linker includes about 3 to about 18 amino acid residues. In aspects, the peptide linker includes about 3 to about 17 amino acid residues. In aspects, the peptide linker includes about 3 to about 16 amino acid residues. In aspects, the peptide linker includes about 3 to about 15 amino acid residues. In aspects, the peptide linker includes about 3 to about 14 amino acid residues. In aspects, the peptide linker includes about 3 to about 13 amino acid residues. In aspects, the peptide linker includes about 3 to about 12 amino acid residues. In aspects, the peptide linker includes about 3 to about 11 amino acid residues. In aspects, the peptide linker includes about 3 to about 10 amino acid residues. In aspects, the peptide linker includes about 3 to about 9 amino acid residues. In aspects, the peptide linker includes about 3 to about 8 amino acid residues. In aspects, the peptide linker includes about 3 to about 7 amino acid residues. In aspects, the peptide linker includes about 3 to about 6 amino acid residues. In aspects, the peptide linker includes about 3 to about 5 amino acid residues. In aspects, the peptide linker includes about 3 to about 4 amino acid residues. In aspects, the peptide linker includes about 10 to about 20 amino acid residues. In aspects, the peptide linker includes about 15 to about 20 amino acid residues. In aspects, the peptide linker includes about 2 amino acid residues. In aspects, the peptide linker includes about 3 amino acid residues. In aspects, the peptide linker includes about 4 amino acid residues. In aspects, the peptide linker includes about 5 amino acid residues. In aspects, the peptide linker includes about 6 amino acid residues. In aspects, the peptide linker includes about 7 amino acid residues. In aspects, the peptide linker includes about 8 amino acid residues. In aspects, the peptide linker includes about 9 amino acid residues. In aspects, the peptide linker includes about 10 amino acid residues. In aspects, the peptide linker includes about 11 amino acid residues. In aspects, the peptide linker includes about 12 amino acid residues. In aspects, the peptide linker includes about 13 amino acid residues. In aspects, the peptide linker includes about 14 amino acid residues. In aspects, the peptide linker includes about 15 amino acid residues. In aspects, the peptide linker includes about 16 amino acid residues. In aspects, the peptide linker includes about 17 amino acid residues. In aspects, the peptide linker includes about 18 amino acid residues. In aspects, the peptide linker includes about 19 amino acid residues. In aspects, the peptide linker includes about 20 amino acid residues. In aspects, the peptide linker includes about 21 amino acid residues. In aspects, the peptide linker includes about 22 amino acid residues. In aspects, the peptide linker includes about 23 amino acid residues. In aspects, the peptide linker includes about 24 amino acid residues. In aspects, the peptide linker includes about 25 amino acid residues.

Examples of linker polypeptides include glycine polymers (G)ₙ, glycine-serine polymers (including, for example, (GS)ₙ, GSGGSₙ (SEQ ID NO: 117), GGSGGSₙ (SEQ ID NO: 118), (GGGGS)n (SEQ ID NO: 119), and GGGSₙ (SEQ ID NO: 120), where n is an integer of at least one, e.g., where n is 1, 2, 3, 4, or 5), glycine-alanine polymers, alanine-serine polymers. Exemplary linkers can comprise amino acid sequences including, but not limited to, GGSG (SEQ ID NO: 121), GGSGG (SEQ ID NO: 122), GSGSG (SEQ ID NO: 123), GSGGG (SEQ ID NO: 124), GGGSG (SEQ ID NO: 125), GSSSG (SEQ ID NO: 126), and the like. The ordinarily skilled artisan will recognize that design of a peptide conjugated to any desired element can include linkers that are all or partially flexible, such that the linker can include a flexible linker as well as one or more portions that confer less flexible structure.

In aspects, the peptide linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to GGSGGGS (SEQ ID NO: 127). In aspects, the peptide linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SGS. In aspects, the peptide linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to EASGSGRASPGIPGSTR (SEQ ID NO: 128). In aspects, the peptide linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to GSG. In aspects, the peptide linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SPG. In aspects, the peptide linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SSGNSNANSRGPSFSSGLVPLSLRGSH (SEQ ID NO: 99). In aspects, the peptide linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to YPYDVPDYA (SEQ ID NO: 129). In embodiments, the peptide linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to ATNFSLLKQAGDVEENPGP (SEQ ID NO: 130)

In some cases, the linker is an XTEN linker. The terms “XTEN,” “XTEN linker,” or “XTEN polypeptide” as used herein refer to a recombinant polypeptide (e.g. unstructured recombinant peptide) lacking hydrophobic amino acid residues. The development and use of XTEN can be found in, for example, Schellenberger et al., Nature Biotechnology 27, 1186-1190 (2009), which is incorporated herein by reference in its entirety and for all purposes. In aspects, the XTEN linker includes about 16 to about 80 amino acid residues. In aspects, the XTEN linker includes about 17 to about 80 amino acid residues. In aspects, the XTEN linker includes about 18 to about 80 amino acid residues. In aspects, the XTEN linker includes about 19 to about 80 amino acid residues. In aspects, the XTEN linker includes about 20 to about 80 amino acid residues. In aspects, the XTEN linker includes about 30 to about 80 amino acid residues. In aspects, the XTEN linker includes about 40 to about 80 amino acid residues. In aspects, the XTEN linker includes about 50 to about 80 amino acid residues. In aspects, the XTEN linker includes about 60 to about 80 amino acid residues. In aspects, the XTEN linker includes about 70 to about 80 amino acid residues. In aspects, the XTEN linker includes about 16 to about 70 amino acid residues. In aspects, the XTEN linker includes about 16 to about 60 amino acid residues. In aspects, the XTEN linker includes about 16 to about 50 amino acid residues. In aspects, the XTEN linker includes about 16 to about 40 amino acid residues. In aspects, the XTEN linker includes about 16 to about 35 amino acid residues. In aspects, the XTEN linker includes about 16 to about 30 amino acid residues. In aspects, the XTEN linker includes about 16 to about 25 amino acid residues. In aspects, the XTEN linker includes about 16 to about 20 amino acid residues. In aspects, the XTEN linker includes about 16 amino acid residues. In aspects, the XTEN linker includes about 17 amino acid residues. In aspects, the XTEN linker includes about 18 amino acid residues. In aspects, the XTEN linker includes about 19 amino acid residues. In aspects, the XTEN linker includes about 20 amino acid residues.

In some embodiments, XTEN linker includes SGSETPGTSESATPES (SEQ ID NO: 131). In aspects, the XTEN linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SGSETPGTSESATPES (SEQ ID NO: 131). In aspects, the XTEN linker includes GSAPGTSTEPSE (SEQ ID NO: 132). In aspects, the XTEN linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to

(SEQ ID NO: 132)
GGPSSGAPPPSGGSPAGSPTSTEEGTSESATPESGPGTSTEPSEG
SAPGSPAGSPTSTEEGTSTEPSEGSAPGTSTEPSE.

Elements of the fusion protein may be arranged in any convenient manner. In embodiments, the fusion protein comprises the structure: A-B-C, or B-A-C or C-A-B, or C-B-A, or B-C-A, or A-C-B; where A comprises a zinc finger DNA binding polypeptide; B comprises a transcriptional repression domain (e.g., KRAB domain), C comprises an epigenome modifying polypeptide (e.g., DNA methyltransferase domain); and wherein the component on the left is the N-terminus and the component on the right is the C-terminus. In aspects, the fusion protein further comprises one or more peptide linkers and one or more detectable tags. In aspects, A-B, B-A, B-C, C-B, A-C, and C-A are each independently linked together via a covalent bond, a peptide linker, a detectable tag, a nuclear localization sequence, or a combination of two or more thereof. The peptide linker can be any peptide linker known in the art (e.g., P2A cleavable peptide, XTEN linker, and the like). In aspects, the fusion protein comprises other components, such as detectable tags (e.g., HA tag, blue fluorescent protein, and the like).

In embodiments, the fusion protein comprises the structure: A-L₁-B-L₂-C, where A comprises a zinc finger DNA binding polypeptide; B comprises a transcriptional repression domain (e.g., KRAB domain), C comprises an epigenome modifying polypeptide (e.g., DNA methyltransferase domain), L₁ is a covalent bond or a peptide linker, and L₂ is a covalent bond or a peptide linker; and where A is at the N-terminus and C is at the C-terminus. In aspects, A is covalently linked to B via a peptide linker. In aspects, A is covalently linked to B via a covalent bond. In aspects, B is covalently linked to C via a peptide linker. In aspects, B is covalently linked to C via a covalent bond. The peptide linker can be any peptide linker known in the art (e.g., P2A cleavable peptide, XTEN linker, and the like). In aspects, the fusion protein comprises other components, such as detectable tags, nuclear localization sequences, and the like. In aspects, L₁ is a covalent bond, a peptide linker, a detectable tag, a nuclear localization sequence, or a combination thereof. In aspects, L₂ is a covalent bond, a peptide linker, a detectable tag, a nuclear localization sequence, or a combination thereof.

In embodiments, the fusion protein comprises the structure: B-L₁-A-L₂-C, where A comprises a zinc finger DNA binding polypeptide; B comprises a transcriptional repression domain (e.g., KRAB domain), C comprises an epigenome modifying polypeptide (e.g., DNA methyltransferase domain), L₁ is a covalent bond or a peptide linker, and L₂ is a covalent bond or a peptide linker; and where B is at the N-terminus and C is at the C-terminus. In aspects, L₁ is a peptide linker. In aspects, L₁ is a covalent bond. In aspects, L₂ is a peptide linker. In aspects, L₂ is a covalent bond. The peptide linker can be any known in the art or described herein (e.g., P2A cleavable peptide, XTEN linker, and the like). In aspects, the fusion protein comprises other components, such as detectable tags. In aspects, L₁ is a covalent bond, a peptide linker, a detectable tag, or a combination thereof. In aspects, L₂ is a covalent bond, a peptide linker, a detectable tag, or a combination thereof. In aspects, the fusion protein further comprises a nuclear localization sequence.

In embodiments, the fusion protein comprises the structure: B-L₃-A-L₄-C-L₅-D; where A comprises a zinc finger DNA binding polypeptide; B comprises a transcriptional repression domain (e.g., KRAB domain), C comprises an epigenome modifying polypeptide (e.g., DNA methyltransferase domain), D is absent or D comprises one or more detectable tags, L₃ comprises a covalent bond, a peptide linker, a detectable tag, or a combination of two or more thereof, L₄ comprises a covalent bond, a peptide linker, a detectable tag, or a combination of two or more thereof, L₅ is absent or L₅ comprises a covalent bond or a peptide linker; and where B is at the N-terminus and D is at the C-terminus. In aspects, L₃ is a peptide linker. In aspects, L₃ is a covalent bond, in aspects, L₃ comprises a peptide linker and a detectable tag. In aspects, L₂ comprises a detectable tag. In aspects, L₄ is a peptide linker. In aspects. L₄ comprises a peptide linker and a detectable tag. In aspects, L₄ is a covalent bond. In aspects. L₄ comprises a detectable tag. In aspects, L₂ is a peptide linker. In aspects, L₅ is a covalent bond. In aspects, D comprises one or a plurality of detectable tags. In aspects, D comprises one detectable tag. In aspects, D comprises two detectable tags. In aspects, D comprises three detectable tags. In aspects, D comprises a plurality of detectable tags. D can be any detectable tag known in the art and/or described herein (e.g., HA tag, blue fluorescent protein, and the like). In aspects L₅ and D are absent. When L₃, L₄, L₅, and D comprise two or more detectable tags, each detectable tag is the same or different. The peptide linker can be any known in the art and/or described herein (e.g., P2A cleavable peptide, XTEN linker, and the like). In aspects, the fusion protein further comprises a nuclear localization sequence.

In embodiments, the fusion protein comprises the structure: C-L₃-A-L₄-B-L₅-D, where A comprises a zinc finger DNA binding polypeptide; B comprises a transcriptional repression domain (e.g., KRAB domain), C comprises an epigenome modifying polypeptide (e.g., DNA methyltransferase domain), D is absent or D comprises one or more detectable tags, L₃ comprises a covalent bond, a peptide linker, a detectable tag, or a combination of two or more thereof, L₄ comprises a covalent bond, a peptide linker, a detectable tag, or a combination of two or more thereof, L₅ is absent or L₅ comprises a covalent bond or a peptide linker; and where C is at the N-terminus and D is at the C-terminus. In aspects, L₃ is a peptide linker. In aspects, L₃ is a covalent bond. In aspects, L₃ comprises a detectable tag. In aspects, L₃ comprises a peptide linker and a detectable tag. In aspects, L₄ a peptide linker. In aspects, L₄ is a covalent bond. In aspects, L₄ comprises a detectable tag. In aspects, L₄ comprises a peptide linker and a detectable tag. In aspects, L₅ a peptide linker. In aspects, L₅ is a covalent bond. In aspects, D comprises One or a plurality of detectable tags. In aspects, D comprises one detectable tag. In aspects, 1D comprises two detectable tags. In aspects, D comprises three detectable tags. In aspects. D comprises a plurality of detectable tags. D can be any detectable tag known in the art and/or described herein (e.g., HA tag, blue fluorescent protein, and the like) In aspects L₅ and D are absent. When L₃, L₄, L₅, and D comprise two or more detectable tags, each detectable tag is the same or different. The peptide linker can be any known in the art and/or described herein (e.g. P2A cleavable peptide, XTEN linker, and the like), in aspects, the fusion protein further comprises a, nuclear localization sequence.

### Nucleic Acids

The fusion protein described herein, including embodiments and aspects thereof, may be provided as a nucleic acid sequence that encodes for the fusion protein. Thus, in an aspect is provided a nucleic acid sequence encoding the fusion protein described herein, including embodiments thereof. In an aspect is provided a nucleic acid sequence encoding the fusion protein described herein (including the DNA-targeting sequence), including embodiments and aspects thereof. In aspects, the nucleic acid sequence encodes for a fusion protein described herein, including fusion proteins having amino acid sequences with certain % sequence identities described herein. In aspects, the nucleic acid is a messenger RNA (mRNA). In aspects, the messenger RNA is messenger RNP. In aspects, the nucleic acid sequence encodes for the fusion proteins described herein, including embodiments and aspects thereof. In addition, present disclosure provides a recombinant expression vector that comprises a nucleotide sequence encoding a fusion protein described herein. In some cases, the nucleotide sequence encoding the fusion polypeptide is operably linked to a promoter that is operable in a cell type of choice (e.g., an HSC or an HPC).

In some embodiments, the nucleic acid of the disclosure encodes for a fusion protein having any one of the zinc finger DNA binding polypeptides presented in Table 1. In some embodiments, a nucleic acid of the disclosure includes the CD81_8 ZFP DNA sequence presented in SEQ ID NO: 65. In some embodiments, a nucleic acid of the disclosure includes the CD81_18 ZFP DNA sequence presented in SEQ ID NO: 66. In some embodiments, a nucleic acid of the disclosure includes the CD81_19 ZFP DNA sequence presented in SEQ ID NO: 67. In some embodiments, a nucleic acid of the disclosure includes the CD81_2 ZFP DNA sequence presented in SEQ ID NO: 68. In some embodiments, a nucleic acid of the disclosure includes the AUNIP_5 ZFP DNA sequence presented in SEQ ID NO: 69. In some embodiments, a nucleic acid of the disclosure includes the AUNIP_14 ZFP DNA sequence presented in SEQ ID NO: 70. In some embodiments, a nucleic acid of the disclosure includes the AUNIP_20 ZFP DNA sequence presented in SEQ ID NO: 71. In some embodiments, a nucleic acid of the disclosure includes the AUNIP_23 ZFP DNA sequence presented in SEQ ID NO: 72. In some embodiments, a nucleic acid of the disclosure includes the PPP6C_7 ZFP DNA sequence presented in SEQ ID NO: 73. In some embodiments, a nucleic acid of the disclosure includes the PPP6C_12 ZFP DNA sequence presented in SEQ ID NO: 74. In some embodiments, a nucleic acid of the disclosure includes the PPP6C_21 ZFP DNA sequence presented in SEQ ID NO: 75. In some embodiments, a nucleic acid of the disclosure includes the PPP6C_24 ZFP DNA sequence presented in SEQ ID NO: 76. In some embodiments, a nucleic acid of the disclosure includes the BAX_19 ZFP DNA sequence presented in SEQ ID NO: 90. In some embodiments, a nucleic acid of the disclosure includes the BAX_23_ZFP ZFP DNA sequence presented in SEQ ID NO: 91. In some embodiments, a nucleic acid of the disclosure includes the BAX_2 ZFP DNA sequence presented in SEQ ID NO: 92.

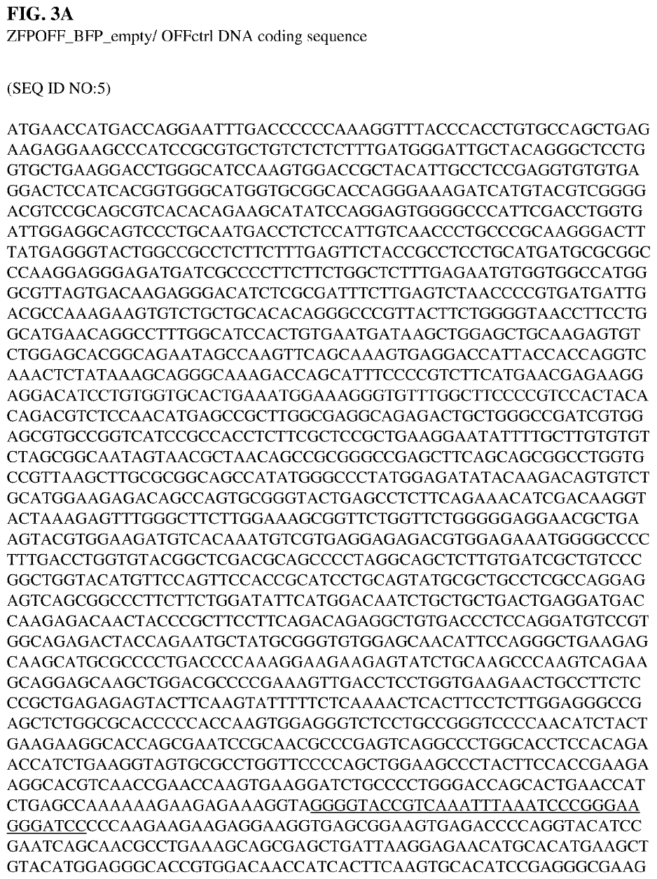

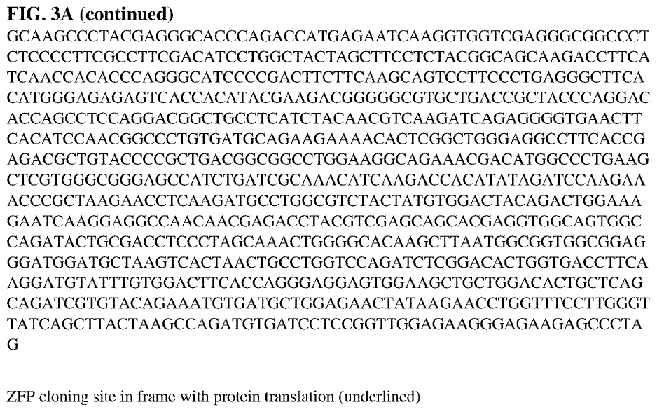

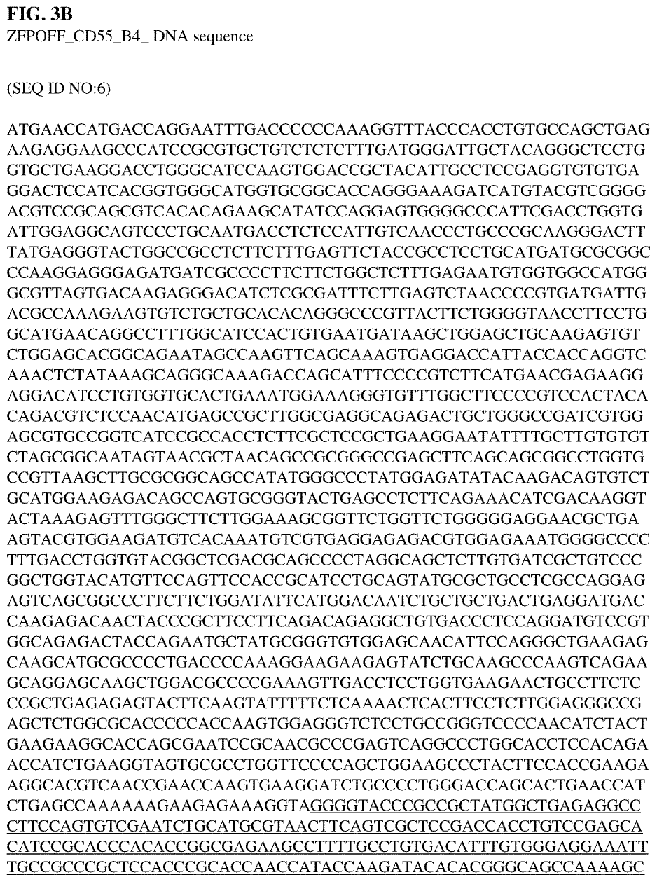

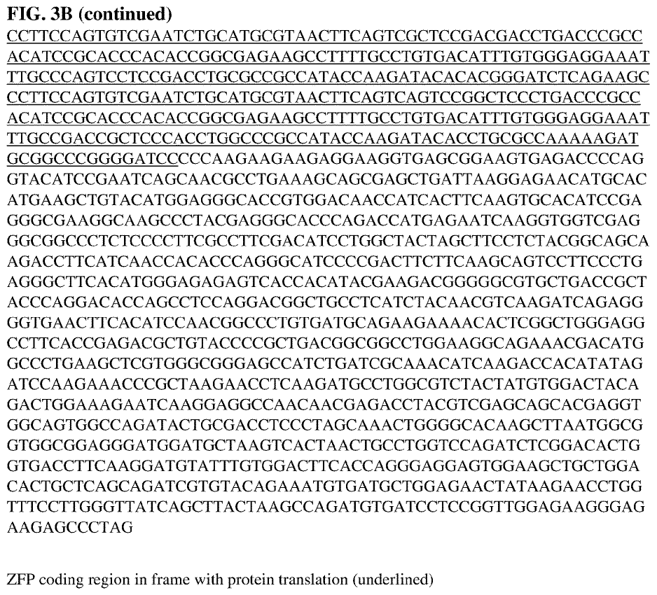

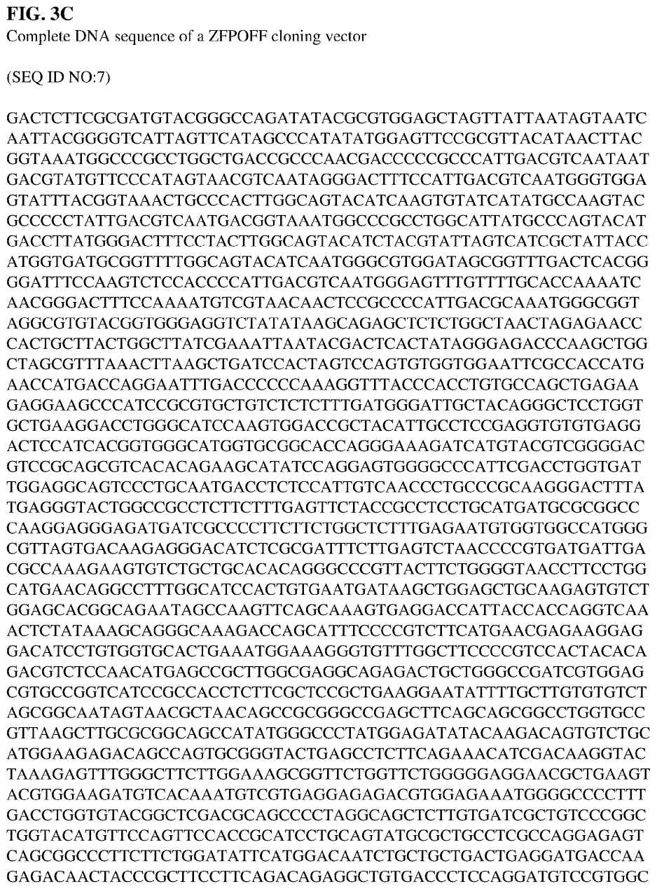

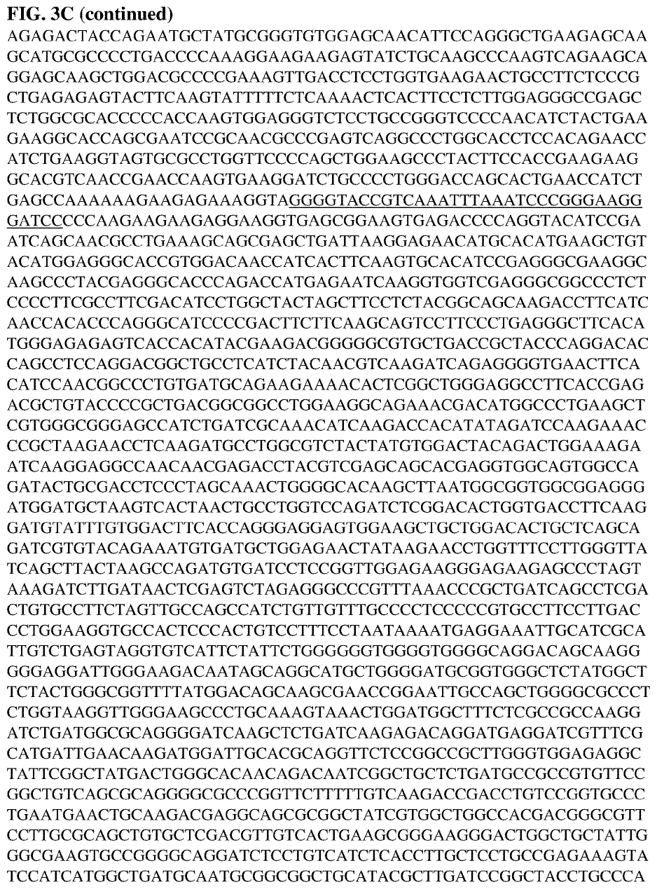

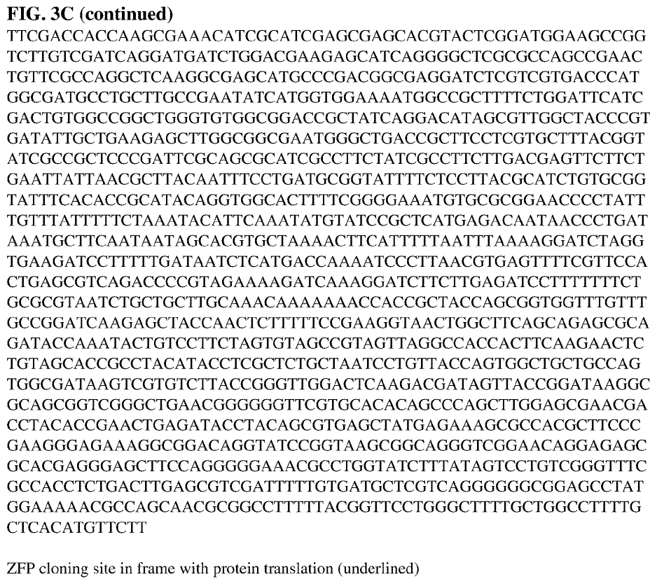

In some cases, the nucleic acid encodes a fusion protein having a ZFP cloning site (e.g., such as those discussed above and presented in FIG. 1A). In such cases, the nucleic acid may include the nucleotide sequence presented in FIG. 3A. As shown in FIG. 3A, the nucleic acid includes a ZFP cloning site in frame with protein translation (underlined).

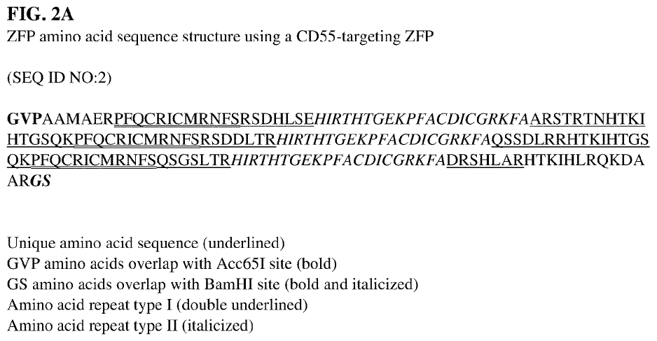

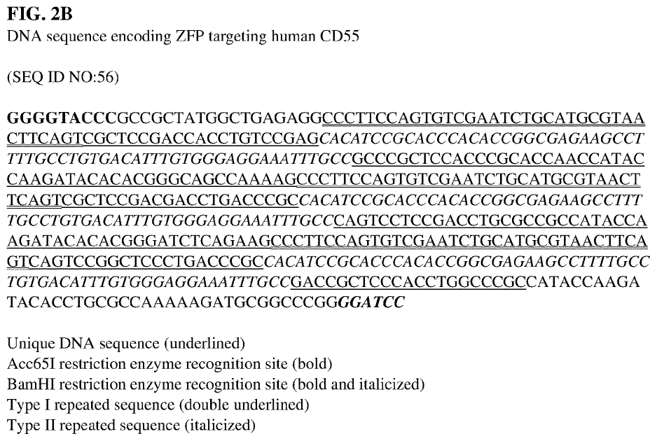

An exemplary nucleic acid sequence encoding a ZFPOFF fusion protein targeting human CD55 is provided in FIG. 3B. The nucleic acid shown in FIG. 3B includes a CD55_B4 ZFP DNA sequence (underlined) presented in SEQ ID NO: 56 and FIG. 2B. In some embodiments, a nucleic acid of the disclosure includes the nucleic acid presented in FIG. 3B.

In some cases, a nucleotide sequence encoding a fusion protein of the present disclosure is codon optimized. This type of optimization can entail a mutation of a fusion protein-encoding nucleotide sequence to mimic the codon preferences of the intended host organism or cell while encoding the same protein. Thus, the codons can be changed, but the encoded protein remains unchanged. For example, if the intended target cell was a human cell, a human codon-optimized fusion protein-encoding nucleotide sequence could be used. As another non-limiting example, if the intended host cell were a mouse cell, then a mouse codon-optimized fusion protein-encoding nucleotide sequence could be generated. As another non-limiting example, if the intended host cell were a plant cell, then a plant codon-optimized fusion protein-encoding nucleotide sequence could be generated. As another non-limiting example, if the intended host cell were an insect cell, then an insect codon-optimized fusion protein-encoding nucleotide sequence could be generated.

It is further contemplated that the nucleic acid sequence encoding the fusion protein as described herein, including embodiments and aspects thereof, may be included in a vector. Therefore, in an aspect is provided a vector including a nucleic acid sequence as described herein, including embodiments and aspects thereof. In aspects, the vector comprises a nucleic acid sequence that encodes for a fusion protein described herein, including fusion proteins having amino acid sequences with certain % sequence identities described herein. Any suitable vector may be employed. In some embodiments, the vector is a viral vector. In other embodiments, the vector is a non-viral vector. Vectors of interest include, but are not limited to, the pVAX1 DNA vaccine vector produced by Thermo Fisher Scientific, and the like.

Suitable expression vectors include viral expression vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (AAV) (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, a recombinant expression vector of the present disclosure is a recombinant adeno-associated virus (AAV) vector. In some cases, a recombinant expression vector of the present disclosure is a recombinant lentivirus vector. In some cases, a recombinant expression vector of the present disclosure is a recombinant retroviral vector.

Depending on the host/vector system utilized, any of a number of suitable transcription and translation control elements, including constitutive and inducible promoters, transcription enhancer elements, transcription terminators, etc. may be used in the expression vector. The transcriptional control element can be a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the transcriptional control element can be functional in eukaryotic cells, e.g., hematopoietic stem cells (e.g., mobilized peripheral blood (mPB) CD34(+) cell, bone marrow (BM) CD34(+) cell, etc.).

Non-limiting examples of eukaryotic promoters (promoters functional in a eukaryotic cell) include EF1α, those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, fluorescent protein, etc.) that can be fused to the fusion protein.

A promoter can be a constitutively active promoter (i.e., a promoter that is constitutively in an active/“ON” state), or it may be an inducible promoter (i.e., a promoter whose state, active/“ON” or inactive/“OFF”, is controlled by an external stimulus, e.g., the presence of a particular temperature, compound, or protein).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

In some cases, a nucleotide sequence encoding a fusion protein of the disclosure is operably linked to (under the control of) a promoter operable in a eukaryotic cell (e.g., a U6 promoter, an enhanced U6 promoter, an H1 promoter, and the like). As would be understood by one of ordinary skill in the art, when expressing an RNA (e.g., a guide RNA) from a nucleic acid (e.g., an expression vector) using a U6 promoter (e.g., in a eukaryotic cell), or another PolIII promoter, the RNA may need to be mutated if there are several Ts in a row (coding for Us in the RNA). This is because a string of Ts (e.g., 5 Ts) in DNA can act as a terminator for polymerase III (PolIII). Thus, in order to ensure transcription of a guide RNA (e.g., the activator portion and/or targeter portion, in dual guide or single guide format) in a eukaryotic cell it may sometimes be necessary to modify the sequence encoding the guide RNA to eliminate runs of Ts. In some cases, a nucleotide sequence encoding a fusion protein of the disclosure is operably linked to a promoter operable in a eukaryotic cell (e.g., a CMV promoter, an EF1α promoter, an estrogen receptor-regulated promoter, and the like).

Examples of inducible promoters include, but are not limited toT7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; estrogen and/or an estrogen analog; IPTG; etc.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Methods of introducing a nucleic acid into a host cell are known in the art, and any convenient method can be used to introduce a nucleic acid (e.g., an expression construct) into a cell. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. In embodiments, the subject nucleic acid is introduced via electroporation.

Introducing the recombinant expression vector into cells can occur in any culture media and under any culture conditions that promote the survival of the cells. Introducing the recombinant expression vector into a target cell can be carried out in vivo or ex vivo. Introducing the recombinant expression vector into a target cell can be carried out in vitro.

In some embodiments, a fusion protein can be provided as RNA. The RNA can be provided by direct chemical synthesis or may be transcribed in vitro from a DNA (e.g., encoding the fusion protein). Once synthesized, the RNA may be introduced into a cell by any of the well-known techniques for introducing nucleic acids into cells (e.g., microinjection, electroporation, transfection, etc.).

Nucleic acids may be provided to the cells using well-developed transfection techniques; see, e.g. Angel and Yanik (2010) PLoS ONE 5(7): e11756, and the commercially available TransMessenger® reagents from Qiagen, Stemfect™ RNA Transfection Kit from Stemgent, and TransIT®-mRNA Transfection Kit from Mirus Bio LLC. See also Beumer et al. (2008) PNAS 105(50):19821-19826.

Vectors may be provided directly to a target host cell. In other words, the cells are contacted with vectors comprising the subject nucleic acids (e.g., recombinant expression vectors) such that the vectors are taken up by the cells. Methods for contacting cells with nucleic acid vectors that are plasmids, include electroporation, calcium chloride transfection, microinjection, and lipofection are well known in the art. For viral vector delivery, cells can be contacted with viral particles comprising the subject viral expression vectors.

Retroviruses, for example, lentiviruses, are suitable for use in methods of the present disclosure. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

Vectors used for providing the nucleic acids encoding a fusion polypeptide to a target host cell can include suitable promoters for driving the expression, that is, transcriptional activation, of the nucleic acid of interest. In other words, in some cases, the nucleic acid of interest will be operably linked to a promoter. This may include ubiquitously acting promoters, for example, the CMV-β-actin promoter, or inducible promoters, such as promoters that are active in particular cell populations or that respond to the presence of drugs such as tetracycline. By transcriptional activation, it is intended that transcription will be increased above basal levels in the target cell by 10 fold or more, by 100 fold or more, or by 1000 fold or more.

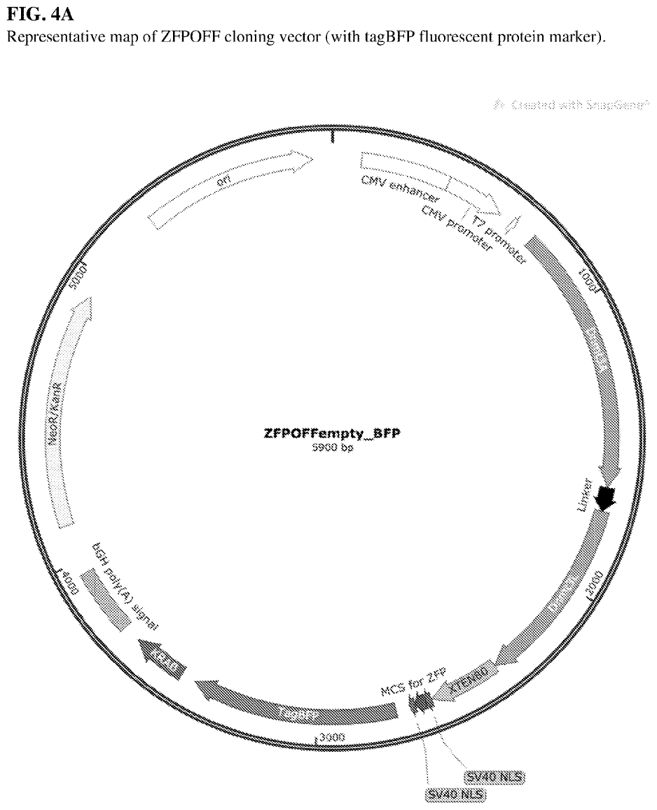

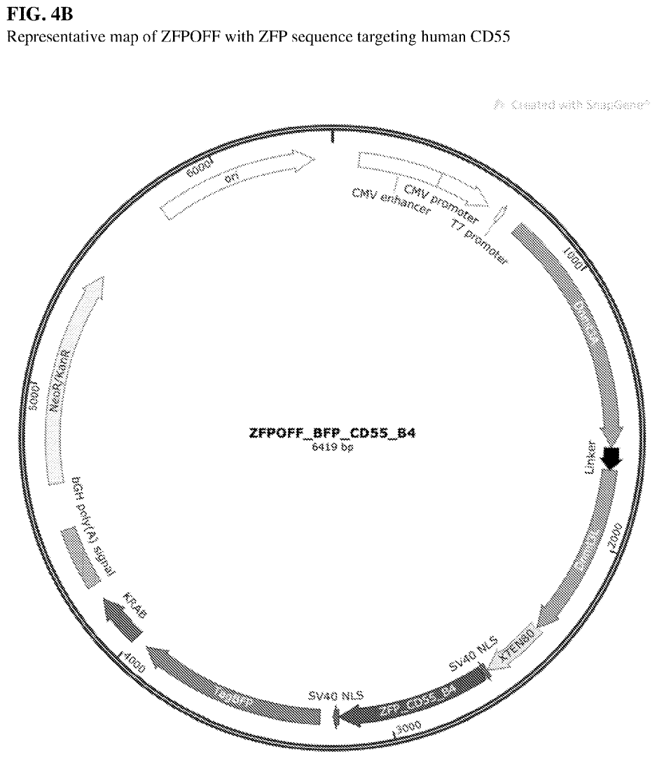

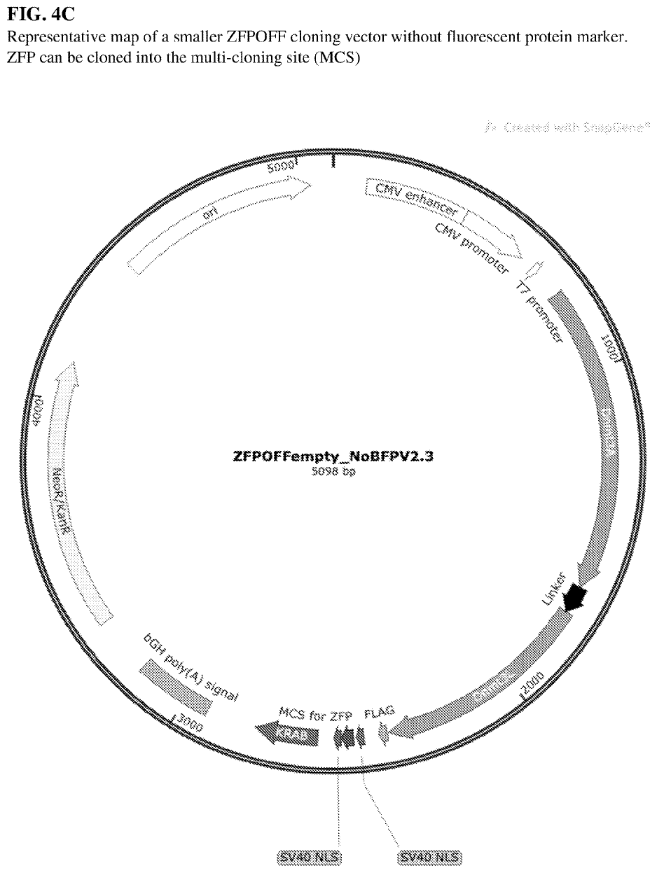

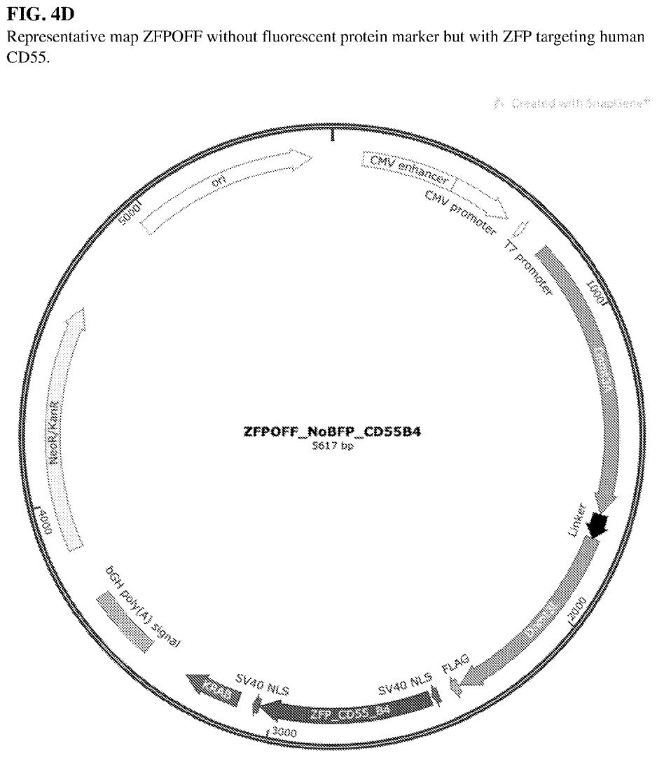

The DNA sequence of an exemplary ZFPOFF cloning vector is provided in FIG. 3C. As shown in FIG. 3C, a ZFP cloning site in frame with protein translation is present in the cloning vector (underlined). In embodiments, a ZFP cloning vector for delivering a nucleic acid encoding the subject fusion protein may have 80% or more sequence identity (e.g., 85% or more, 90% or more, 95% or more, 97% or more, 98% or more, 99% or more, or 100% sequence identity) with the nucleic acid sequence presented in FIG. 3C. Representative maps of exemplary cloning vectors are shown in FIG. 4A-4C. A representative map of a ZFPOFF cloning vector having a tagBFP fluorescent protein marker is shown in FIG. 4A. A representative map of a ZFPOFF cloning vector with a ZFP sequence targeting human CD55 is depicted in FIG. 4B. A Representative map of a smaller ZFPOFF cloning vector without the fluorescent protein marker is shown in FIG. 4C. The cloning vector of FIG. 4C includes a multi-cloning site (MCS) that facilitates the cloning of a nucleic acid encoding a ZFP DNA binding polypeptide (e.g., such as those presented in Table 1).

A nucleic acid comprising a nucleotide sequence encoding a fusion polypeptide, is in some cases an RNA. Thus, a fusion protein can be introduced into cells as RNA. Methods of introducing RNA into cells are known in the art and may include, for example, direct injection, transfection, or any other method used for the introduction of DNA. A fusion protein may instead be provided to cells as a polypeptide. Such a polypeptide may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like. The polypeptide may be formulated for improved stability. For example, the peptides may be PEGylated, where the polyethyleneoxy group provides for enhanced lifetime in the blood stream.

Additionally, or alternatively, a fusion polypeptide of the present disclosure may be fused to a polypeptide permeant domain to promote uptake by the cell. A number of permeant domains are known in the art and may be used in the non-integrating polypeptides of the present disclosure, including peptides, peptidomimetics, and non-peptide carriers. For example, a permeant peptide may be derived from the third alpha helix of Drosophila melanogaster transcription factor Antennapedia, referred to as penetratin, which comprises the amino acid sequence RQIKIWFQNRRMKWKK (SEQ ID NO: 133). As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-9 and 446; and Wender et al. (2000) Proc. Natl. Acad. Sci. U.S.A 2000 Nov. 21; 97(24):13003-8; published U.S. patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002). The site at which the fusion is made may be selected in order to optimize the biological activity, secretion or binding characteristics of the polypeptide. The optimal site will be determined by routine experimentation.

A fusion polypeptide of the present disclosure may be produced in vitro or by eukaryotic cells or by prokaryotic cells, and it may be further processed by unfolding, e.g. heat denaturation, dithiothreitol reduction, etc. and may be further refolded, using methods known in the art.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also suitable for inclusion in embodiments of the present disclosure are nucleic acids and proteins that have been modified using ordinary molecular biological techniques and synthetic chemistry so as to improve their resistance to proteolytic degradation, to change the target sequence specificity, to optimize solubility properties, to alter protein activity (e.g., transcription modulatory activity, enzymatic activity, etc.) or to render them more suitable. Analogs of such polypeptides include those containing residues other than naturally occurring L-amino acids, e.g. D-amino acids or non-naturally occurring synthetic amino acids. D-amino acids may be substituted for some or all of the amino acid residues.

A fusion polypeptide of the present disclosure may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus, cysteines can be used to make thioethers, histidines for linking to a metal ion complex, carboxyl groups for forming amides or esters, amino groups for forming amides, and the like.

A fusion polypeptide of the present disclosure may also be isolated and purified in accordance with conventional methods of recombinant synthesis. A lysate may be prepared of the expression host and the lysate purified using high performance liquid chromatography (HPLC), exclusion chromatography, gel electrophoresis, affinity chromatography, or other purification technique. For the most part, the compositions which are used will comprise 20% or more by weight of the desired product, more usually 75% or more by weight, preferably 95% or more by weight, and for therapeutic purposes, usually 99.5% or more by weight, in relation to contaminants related to the method of preparation of the product and its purification. Usually, the percentages will be based upon total protein. Thus, in some cases a fusion polypeptide of the present disclosure is at least 80% pure, at least 85% pure, at least 90% pure, at least 95% pure, at least 98% pure, or at least 99% pure (e.g., free of contaminants, non-fusion proteins or other macromolecules, etc.).

To improve the delivery of a DNA vector into a target cell, the DNA can be protected from damage and its entry into the cell facilitated, for example, by using lipoplexes and polyplexes. Thus, in some cases, a nucleic acid of the present disclosure (e.g., a recombinant expression vector of the present disclosure) can be covered with lipids in an organized structure like a micelle or a liposome. When the organized structure is complexed with DNA it is called a lipoplex. There are three types of lipids, anionic (negatively-charged), neutral, or cationic (positively-charged). Lipoplexes that utilize cationic lipids have proven utility for gene transfer. Cationic lipids, due to their positive charge, naturally complex with the negatively charged DNA. Also as a result of their charge, they interact with the cell membrane. Endocytosis of the lipoplex then occurs, and the DNA is released into the cytoplasm. The cationic lipids also protect against degradation of the DNA by the cell.

Complexes of polymers with DNA are called polyplexes. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis) such as inactivated adenovirus must occur. However, this is not always the case; polymers such as polyethylenimine have their own method of endosome disruption as does chitosan and trimethylchitosan.

Dendrimers, a highly branched macromolecule with a spherical shape, may also be used to genetically modify stem cells. The surface of the dendrimer particle may be functionalized to alter its properties. In particular, it is possible to construct a cationic dendrimer (i.e., one with a positive surface charge). When in the presence of genetic material such as a DNA plasmid, charge complementarity leads to a temporary association of the nucleic acid with the cationic dendrimer. On reaching its destination, the dendrimer-nucleic acid complex can be taken up into a cell by endocytosis.

A possible modification of a subject nucleic acid involves chemically linking to the polynucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

A conjugate may include a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle (e.g., the nucleus). In some embodiments, a PTD is covalently linked to the 3′ end of an exogenous polynucleotide. In some embodiments, a PTD is covalently linked to the 5′ end of an exogenous polynucleotide. Exemplary PTDs include but are not limited to a minimal undecapeptide protein transduction domain (corresponding to residues 47-57 of HIV-1 TAT comprising YGRKKRRQRRR; SEQ ID NO: 134); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR SEQ ID NO: 135); Transportan GWTLNSAGYLLGKINLKALAALAKKIL SEQ ID NO: 136); KALAWEAKLAKALAKALAKHLAKALAKALKCEA SEQ ID NO: 137); and RQIKIWFQNRRMKWKK SEQ ID NO: 133). Exemplary PTDs include but are not limited to, YGRKKRRQRRR SEQ ID NO: 134), RKKRRQRRR SEQ ID NO: 138); an arginine homopolymer of from 3 arginine residues to 50 arginine residues; Exemplary PTD domain amino acid sequences include, but are not limited to, any of the following: YGRKKRRQRRR SEQ ID NO: 134); RKKRRQRR SEQ ID NO: 139); YARAAARQARA SEQ ID NO: 140); THRLPRRRRRR SEQ ID NO: 141); and GGRRARRRRRR SEQ ID NO: 142). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

Introducing Components into a Target Cell

A fusion polypeptide of the present disclosure (or a nucleic acid that includes a nucleotide sequence encoding a fusion polypeptide of the present disclosure) can be introduced into a host cell by any of a variety of well-known methods. Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13, pii: S0169-409X(12)00283-9. doi: 10.1016/j.addr.2012.09.023), and the like.

To induce the desired modification to a target nucleic acid (e.g., genomic DNA), or any desired modification to a polypeptide associated with target nucleic acid, the fusion protein, whether introduced as nucleic acids or polypeptides, are provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The agent(s) may be provided to the subject cells one or more times, e.g. one time, twice, three times, or more than three times, and the cells allowed to incubate with the agent(s) for some amount of time following each contacting event e.g. 16-24 hours, after which time the media is replaced with fresh media and the cells are cultured further.

In some cases, a fusion polypeptide of the present disclosure is provided as a nucleic acid (e.g., an mRNA, a DNA, a plasmid, an expression vector, a viral vector, etc.) that encodes the fusion polypeptide. In some cases, the fusion polypeptide of the present disclosure is provided directly as a protein (e.g., without an associated guide RNA or with an associate guide RNA, i.e., as a ribonucleoprotein complex). A fusion polypeptide of the present disclosure can be introduced into a cell (provided to the cell) by any convenient method; such methods are known to those of ordinary skill in the art. As an illustrative example, a fusion polypeptide of the present disclosure can be injected directly into a cell. As another example, a fusion polypeptide of the present disclosure can be introduced into a cell (e.g., eukaryotic cell) (e.g., via injection, via nucleofection; via a protein transduction domain (PTD) conjugated to one or more components, e.g., conjugated to the fusion protein, etc.).

In some cases, a nucleic acid (e.g., a nucleic acid comprising a nucleotide sequence encoding a fusion polypeptide of the present disclosure; etc.) is delivered to a cell (e.g., a target host cell) in a particle, or associated with a particle. The terms “particle” and “nanoparticle” can be used interchangeably, as appropriate. A recombinant expression vector comprising a nucleotide sequence encoding a fusion polypeptide of the present disclosure and/or an mRNA comprising a nucleotide sequence encoding a fusion polypeptide of the present disclosure may be delivered simultaneously using particles or lipid envelopes; for instance, a fusion polypeptide can be delivered via a particle, e.g., a delivery particle comprising lipid or lipidoid and hydrophilic polymer, e.g., a cationic lipid and a hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG); and/or wherein the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DMPC 0, PEG 5, Cholesterol 5). In addition, a biodegradable core-shell structured nanoparticle with a poly(β-amino ester) (PBAE) core enveloped by a phospholipid bilayer shell can be used. In some cases, particles/nanoparticles based on self-assembling bioadhesive polymers are used; such particles/nanoparticles may be applied to oral delivery of peptides, intravenous delivery of peptides and nasal delivery of peptides, e.g., to the brain. Other embodiments, such as oral absorption and ocular delivery of hydrophobic drugs are also contemplated. A molecular envelope technology, which involves an engineered polymer envelope which is protected and delivered to the site of the disease, can be used. Doses of about 5 mg/kg can be used, with single or multiple doses, depending on various factors, e.g., the target tissue.

Lipidoid compounds (e.g., as described in US patent application 20110293703) are also useful in the administration of polynucleotides, and can be used to deliver a fusion polypeptide and/or nucleic acid of the present disclosure. A poly(beta-amino alcohol) (PBAA) can be used to deliver a fusion polypeptide of the present disclosure. US Patent Publication No. 20130302401 relates to a class of poly(beta-amino alcohols) (PBAAs) that has been prepared using combinatorial polymerization. Sugar-based particles may be used, for example GalNAc, as described with reference to WO2014118272 (incorporated herein by reference) and Nair, J K et al., 2014, Journal of the American Chemical Society 136 (49), 16958-16961) can be used to deliver a fusion polypeptide of the present disclosure to a target cell.

In some cases, lipid nanoparticles (LNPs) are used to deliver a fusion polypeptide and/or nucleic acid of the present disclosure to a target cell. Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). Preparation of LNPs and is described in, e.g., Rosin et al. (2011) Molecular Therapy 19:1286-2200). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), and R-3-[(.omega.-methoxy-poly(ethylene glycol)2000) carbamoyl]-1,2-dimyristyloxlpropyl-3-amine (PEG-C-DOMG) may be used. A nucleic acid may be encapsulated in LNPs containing DLinDAP, DLinDMA, DLinK-DMA, and DLinKC2-DMA (cationic lipid:DSPC:CHOL:PEGS-DMG or PEG-C-DOMG at 40:10:40:10 molar ratios). In some cases, 0.2% SP-DiOC18 is incorporated.

Spherical Nucleic Acid (SNA™) constructs and other nanoparticles (particularly gold nanoparticles) can be used to deliver a fusion polypeptide and/or nucleic acid of the present disclosure to a target cell. See, e.g., Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19): 7625-7630, Jensen et al., Sci. Transl. Med. 5, 209ra152 (2013) and Mirkin, et al., Small, 10:186-192.

In general, a “nanoparticle” refers to any particle having a diameter of less than 1000 nm. In some cases, nanoparticles suitable for use in delivering a fusion polypeptide and/or nucleic acid of the present disclosure to a target cell have a diameter of 500 nm or less, e.g., from 25 nm to 35 nm, from 35 nm to 50 nm, from 50 nm to 75 nm, from 75 nm to 100 nm, from 100 nm to 150 nm, from 150 nm to 200 nm, from 200 nm to 300 nm, from 300 nm to 400 nm, or from 400 nm to 500 nm. In some cases, nanoparticles suitable for use in delivering a fusion polypeptide of the present disclosure to a target cell have a diameter of from 25 nm to 200 nm. In some cases, nanoparticles suitable for use in delivering a fusion polypeptide of the present disclosure to a target cell have a diameter of 100 nm or less.

Nanoparticles suitable for use in delivering a fusion polypeptide and/or nucleic acid to a target cell may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically below 10 nm) that quantization of electronic energy levels occurs. Such nanoscale particles are used in biomedical applications as drug carriers or imaging agents and may be adapted for similar purposes in the present disclosure.

Semi-solid and soft nanoparticles are also suitable for use in delivering a fusion polypeptide and/or nucleic acid of the present disclosure, to a target cell. A prototype nanoparticle of semi-solid nature is the liposome. In some cases, a liposome is used to deliver a fusion polypeptide and/or nucleic acid of the present disclosure to a target cell. Liposomes are spherical vesicle structures composed of a uni- or multilamellar lipid bilayer surrounding internal aqueous compartments and a relatively impermeable outer lipophilic phospholipid bilayer. Liposomes can be made from several different types of lipids; however, phospholipids are most commonly used to generate liposomes. Although liposome formation is spontaneous when a lipid film is mixed with an aqueous solution, it can also be expedited by applying force in the form of shaking by using a homogenizer, sonicator, or an extrusion apparatus. Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. A liposome formulation may be mainly comprised of natural phospholipids and lipids such as 1,2-distearoryl-sn-glycero-3-phosphatidyl choline (DSPC), sphingomyelin, egg phosphatidylcholines and monosialoganglioside.

A stable nucleic-acid-lipid particle (SNALP) can be used to deliver a fusion polypeptide and/or nucleic acid of the present disclosure to a target cell. The SNALP formulation may contain the lipids 3-N-[(methoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio. The SNALP liposomes may be prepared by formulating D-Lin-DMA and PEG-C-DMA with distearoylphosphatidylcholine (DSPC), Cholesterol and RNA using a 25:1 lipid/RNA ratio and a 48/40/10/2 molar ratio of Cholesterol/D-Lin-DMA/DSPC/PEG-C-DMA. The resulting SNALP liposomes can be about 80-100 nm in size. A SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol)2000)carbamoyl]-1,2-dimyrestyloxypropylamine, and cationic 1,2-dilinoleyloxy-3-N,Ndimethylaminopropane. A SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinoleyloxy-3-(N; N-dimethyl)aminopropane (DLinDMA).

Other cationic lipids, such as amino lipid 2,2-dilinoleyl-4-dimethylaminoethyl-[1,3]-dioxolane (DLin-KC2-DMA) can be used to deliver a fusion polypeptide and/or nucleic acid of the present disclosure to a target cell. A preformed vesicle with the following lipid composition may be contemplated: amino lipid, distearoylphosphatidylcholine (DSPC), cholesterol and (R)-2,3-bis(octadecyloxy) propyl-1-(methoxy poly(ethylene glycol)2000)propylcarbamate (PEG-lipid) in the molar ratio 40/10/40/10, respectively, and an RNA/total lipid ratio of approximately 0.05 (w/w). To ensure a narrow particle size distribution in the range of 70-90 nm and a low polydispersity index of 0.11.+−0.0.04 (n=56), the particles may be extruded up to three times through 80 nm membranes prior to adding the guide RNA. Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

Supercharged proteins can be used to deliver a fusion polypeptide and/or nucleic acid of the present disclosure to a target cell. Supercharged proteins are a class of engineered or naturally occurring proteins with unusually high positive or negative net theoretical charge. Both supernegatively and superpositively charged proteins exhibit the ability to withstand thermally or chemically induced aggregation. Superpositively charged proteins are also able to penetrate mammalian cells. Associating cargo with these proteins, such as plasmid DNA, RNA, or other proteins, can facilitate the functional delivery of these macromolecules into mammalian cells both in vitro and in vivo.

Cell Penetrating Peptides (CPPs) can be used to deliver a fusion polypeptide and/or nucleic acid of the present disclosure to a target cell. CPPs typically have an amino acid composition that either contains a high relative abundance of positively charged amino acids such as lysine or arginine or has sequences that contain an alternating pattern of polar/charged amino acids and non-polar, hydrophobic amino acids.

An implantable device can be used to deliver a fusion polypeptide and/or nucleic acid of the present disclosure to a target cell (e.g., a target cell in vivo, where the target cell is a target cell in circulation, a target cell in a tissue, a target cell in an organ, etc.). An implantable device suitable for use in delivering a fusion polypeptide and/or nucleic acid of the present disclosure to a target cell (e.g., a target cell in vivo, where the target cell is a target cell in circulation, a target cell in a tissue, a target cell in an organ, etc.) can include a container (e.g., a reservoir, a matrix, etc.) that comprises the fusion polypeptide(or component thereof, e.g., a nucleic acid of the present disclosure).

A suitable implantable device can comprise a polymeric substrate, such as a matrix for example, that is used as the device body, and in some cases additional scaffolding materials, such as metals or additional polymers, and materials to enhance visibility and imaging. An implantable delivery device can be advantageous in providing release locally and over a prolonged period, where the polypeptide and/or nucleic acid to be delivered is released directly to a target site, e.g., the extracellular matrix (ECM), the vasculature surrounding a tumor, a diseased tissue, etc. Suitable implantable delivery devices include devices suitable for use in delivering to a cavity such as the abdominal cavity and/or any other type of administration in which the drug delivery system is not anchored or attached, comprising a biostable and/or degradable and/or bioabsorbable polymeric substrate, which may for example optionally be a matrix. In some cases, a suitable implantable drug delivery device comprises degradable polymers, wherein the main release mechanism is bulk erosion. In some cases, a suitable implantable drug delivery device comprises non degradable, or slowly degraded polymers, wherein the main release mechanism is diffusion rather than bulk erosion, so that the outer part functions as membrane, and its internal part functions as a drug reservoir, which practically is not affected by the surroundings for an extended period (for example from about a week to about a few months). Combinations of different polymers with different release mechanisms may also optionally be used. The concentration gradient at the can be maintained effectively constant during a significant period of the total releasing period, and therefore the diffusion rate is effectively constant (termed “zero mode” diffusion). By the term “constant” it is meant a diffusion rate that is maintained above the lower threshold of therapeutic effectiveness, but which may still optionally feature an initial burst and/or may fluctuate, for example increasing and decreasing to a certain degree. The diffusion rate can be so maintained for a prolonged period, and it can be considered constant to a certain level to optimize the therapeutically effective period, for example the effective silencing period. In some cases, the implantable delivery system is designed to shield the nucleotide based therapeutic agent from degradation, whether chemical in nature or due to attack from enzymes and other factors in the body of the subject. The site for implantation of the device, or target site, can be selected for maximum therapeutic efficacy.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A fusion protein comprising: a) a zinc finger DNA binding polypeptide comprising an alpha-helix recognition domain configured to bind to a target nucleotide sequence in a target nucleic acid, wherein the target nucleic acid is selected from a target CD81 nucleic acid, a target BAX nucleic acid, a target PPP6C nucleic acid, and a target AUNIP nucleic acid; and b) an epigenome modifying polypeptide.

Aspect 2. The fusion protein according to Aspect 1, wherein the epigenome modifying polypeptide comprises a DNA methyltransferase domain.

Aspect 3. The fusion protein according to Aspect 2, wherein the DNA methyltransferase polypeptide comprises a DNA methyltransferase 3 alpha (DNMT3A) domain.

Aspect 4. The fusion protein according to Aspect 2, wherein the DNA methyltransferase polypeptide comprises a DNA methyltransferase 3 like (DNMT3L) domain.

Aspect 5. The fusion protein according to any of Aspects 2 to 4, wherein the DNA methyltransferase polypeptide is a DNMT3A-3L polypeptide.

Aspect 6. The fusion protein according to any of the preceding aspects, wherein the fusion protein comprises, from N-terminus to C-terminus, the epigenome modifying polypeptide and the zinc finger DNA binding polypeptide.

Aspect 7. The fusion protein according to any of the preceding aspects, wherein the zinc finger DNA binding polypeptide and the epigenome modifying polypeptide are separated by a linker.

Aspect 8. The fusion protein according to Aspect 7, wherein the linker is an XTEN linker.

Aspect 9. The fusion protein according to Aspect 8, wherein the XTEN linker comprises 80 amino acids.

Aspect 10. The fusion protein according to any of the preceding aspects, further comprising a transcriptional repression polypeptide.

Aspect 11. The fusion protein according to Aspect 10, wherein the transcriptional repression domain is a Kruppel associated box (KRAB) polypeptide.

Aspect 12. The fusion protein according to Aspect 10 or 11, wherein the fusion protein comprises, from N-terminus to C-terminus, the epigenome modifying polypeptide, the zinc finger DNA binding polypeptide, and the transcriptional repression polypeptide.

Aspect 13. The fusion protein according to any one of Aspects 10 of 12, wherein the epigenome modifying polypeptide and the transcriptional repression polypeptide are separated by a linker.

Aspect 14. The fusion protein according to Aspect 13, wherein the linker is a an XTEN linker.

Aspect 15. The fusion protein according to Aspect 14, wherein the XTEN linker comprises 16 amino acids.

Aspect 16. The fusion protein according to any of the preceding aspects, further comprising a nuclear localization signal (NLS).

Aspect 17, A nucleic acid comprising a nucleotide sequence encoding the fusion protein of any one of Aspects 1-16.

Aspect 18. The nucleic acid according to Aspect 17, wherein the nucleotide sequence is operably linked to a promoter.

Aspect 19. The nucleic acid according to Aspect 18, wherein the promoter is functional in a hematopoietic stem cell (HSC) or a hematopoietic progenitor cell (HPC).

Aspect 20. A recombinant expression vector comprising the nucleic acid of any of Aspects 17 to 19.

Aspect 21. The recombinant expression vector according to Aspect 20, wherein the recombinant expression vector is a recombinant adenoassociated viral vector, a recombinant retroviral vector, or a recombinant lentiviral vector.

Aspect 22. A hematopoietic stem cell (HSC) or a hematopoietic progenitor cell (HPC) comprising one or more of: (a) the fusion protein according to any of Aspects 1 to 16; (b) the nucleic acid according to any of Aspects 17 to 19; and (c) the recombinant expression vector according to Aspect 20 or Aspect 21.

Aspect 23. The HSC or HPC according to Aspect 22, wherein the HSC is a human HSC or wherein the HPC is a human HPC.

Aspect 24. The HSC or HPC according to Aspect 22 or Aspect 23, wherein the nucleic acid is integrated into the genomic DNA of the HSC or the HPC.

Aspect 25. A method of reducing ionizing radiation-induced damage to a hematopoietic stem cell (HSC) or a hematopoietic progenitor cell (HPC) in an individual, the method comprising introducing into the HSC or the HPC: (a) the fusion protein according to any of Aspects 1 to 16; (b) the nucleic acid according to any of Aspects 17 to 19; or (c) the recombinant expression vector according to Aspect 20 or Aspect 21.

Aspect 26. The method of aspect 25, wherein the ionizing radiation is administered for the treatment of a cancer.

Aspect 27. The method of aspect 26, wherein the cancer is selected from small cell lung cancer, triple-negative breast cancer, human papilloma virus (HPV)-positive head and neck cancer, retinoblastoma, retinoblastoma protein (Rb)-negative bladder cancer, retinoblastoma protein (Rb)-negative prostate cancer, osteosarcoma, ovarian cancer, uterine cancer, and cervical cancer.

Aspect 28. The method of aspect 26 or aspect 27, wherein the fusion protein, the nucleic acid, or the recombinant expression vector is administered from 1 hour to 48 hours before the ionizing radiation treatment for the cancer.

Aspect 29. The method of aspect 25, wherein the ionizing radiation occurs through an occupational, environmental, disaster, terrorist, or warfare event.

Aspect 30. The method of any one of aspects 25-29, comprising administering to the individual: (a) the fusion protein according to any of Aspects 1 to 16; (b) the nucleic acid according to any of Aspects 17 to 19; or (c) the recombinant expression vector according to Aspect 20 or Aspect 21.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

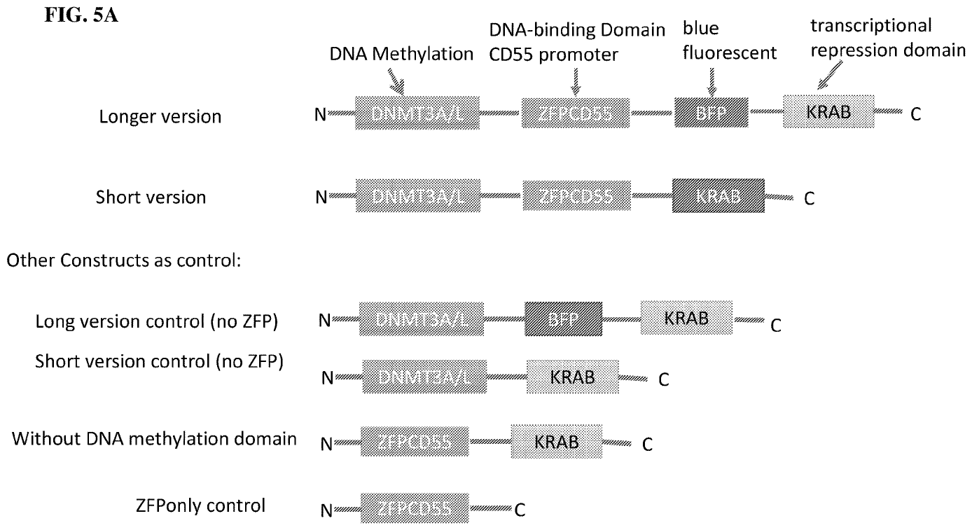

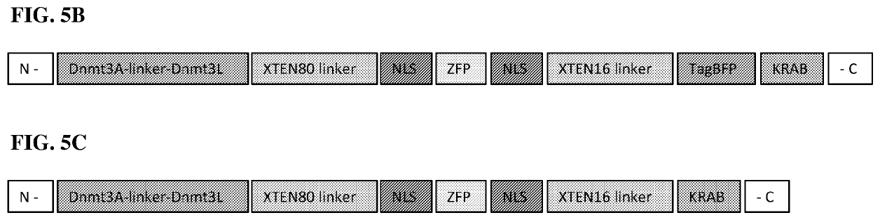

In order to investigate the role of blue fluorescent protein (BFP) in gene repression by fusion proteins having a zinc finger DNA binding polypeptide and an epigenome modifying polypeptide (referred to as “ZFPoff”), constructs having one or more of the following domains were created: a DNA methylation domain, a DNA binding domain, a blue fluorescent protein domain and a transcriptional repression domain. FIG. 5A presents the different constructs that were tested. The “longer version” included, from N-terminus to C-terminus, a DNA methylation domain, a DNA binding domain, a blue fluorescent protein domain and a transcriptional repression domain. The “short version” was the same, but did not include a blue fluorescent protein domain. Controls of the long and short versions that did not have the ZFPCD55 DNA binding domain were also created and are depicted in FIG. 5A. One construct lacked the DNA methylation domain but included a transcriptional repression domain, and another construct was included that only had the DNA binding domain to test if ZFP itself can repress gene transcription. Alternate depictions of select constructs are shown in FIG. 5B-5C. Constructs having a zinc finger DNA binding polypeptide were included to determine whether ZFP itself can repress gene transcription. Instead of lentiviral delivery, each construct plasmid was electroporated to a K562 leukemia cancer cell line. The amount of DNA was normalized to 1 μg, and the construct DNA were plasmids expressed transiently.

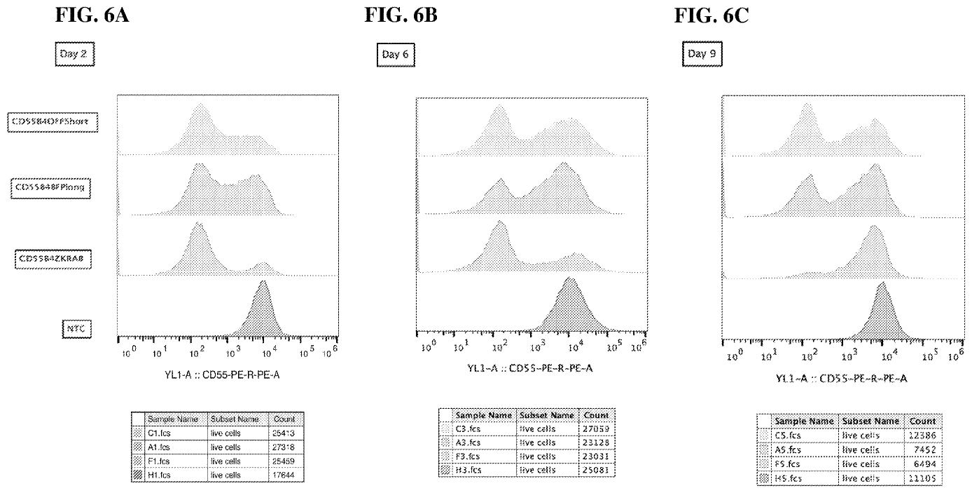

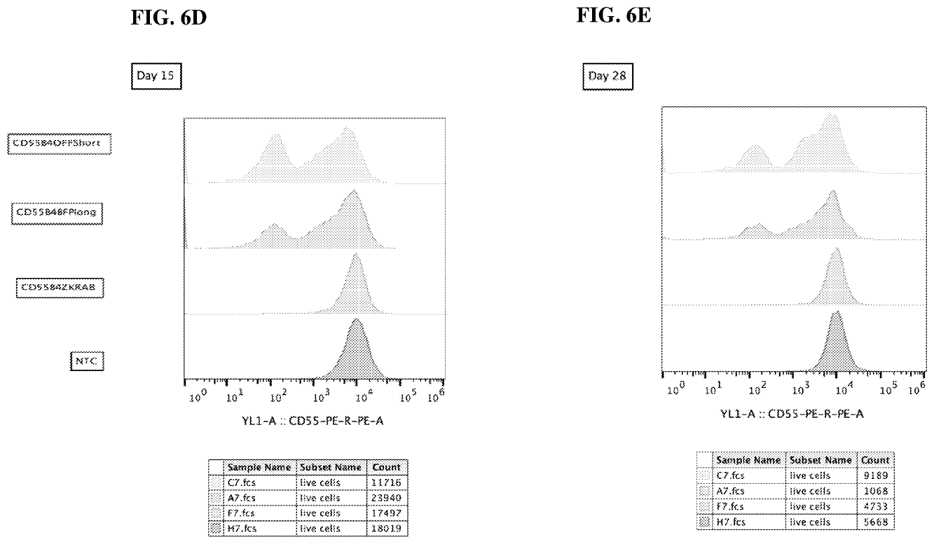

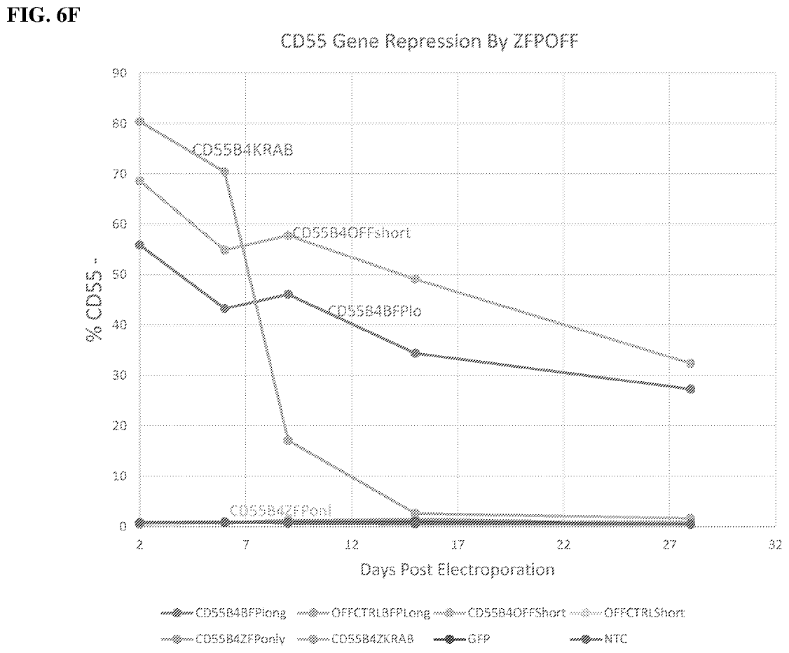

As shown in FIG. 6A-6E, CD55 repression was measured at each of several time points following transient expression (i.e., day 2, day 6, day 9, day 15 and day 28). Cells with the shorter version (top) were better at repressing CD55 marker, with a slightly more CD55− population than the cells with longer version construct (second from top). The cells with KRAB construct (third from top) were better at repressing CD55 marker but have a short repression time as can be seen from the majority of the CD55− population shifts back to CD55+ on day 9 (FIG. 6C). The bottom graphs represent a control where CD55 was not repressed. Even a month after the transient expression, a portion of the cells still retained CD55 repression.

A line graph comparing CD55 Gene Repression among the different constructs is presented in FIG. 6F. As shown in FIG. 6F, the shorter version worked well at repressing CD55. This suggests that BFP is not essential for the function of ZFPoff. The construct only having ZFP (“ZFPonly”) does not appear to have effect on CD55 expression. This suggests that ZFP on its own does not repress gene expression.

### Example 2

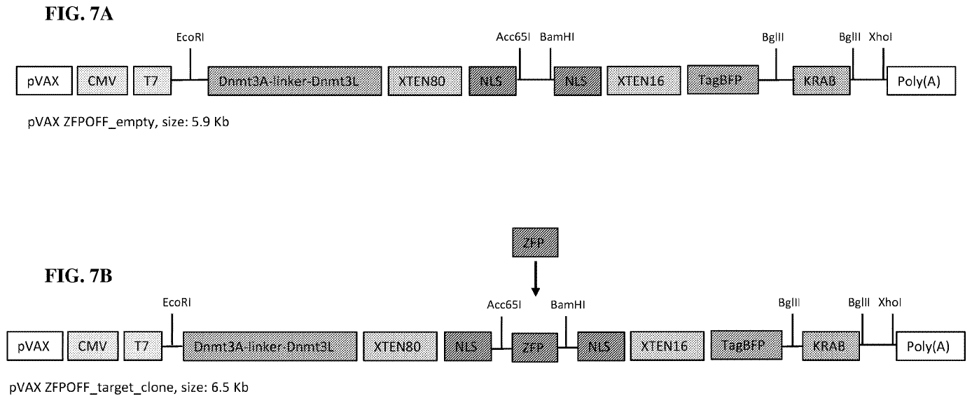

A DNA sequence encoding Dnmt3A and Dnmt3L DNA methyltransferase domains, a multi-cloning site (MCS) in frame with translation, a blue fluorescent protein marker (BFP) and a KRAB repressor domain were cloned into the pVAX1 vector between EcoRI and BglII restriction sites (ZFPOFF_empty; FIG. 7A). DNA sequences encoding ZFP were cut from a vector (Sigma-Aldrich), cloned in the MCS using Acc65I and BamHI restriction enzyme digest and ligated (FIG. 7B).

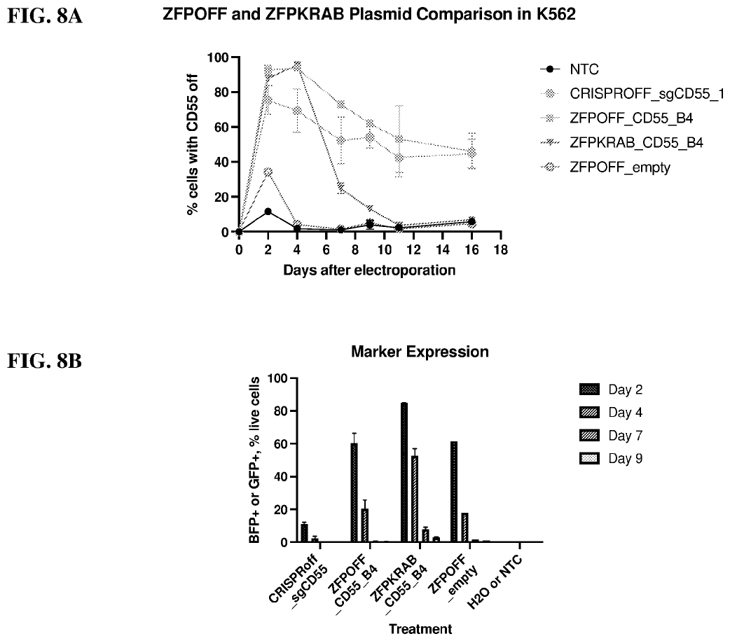

ZFP were purchased from Sigma-Aldrich (Millipore) and delivered as plasmids on Sigma's pER-GFP-KRAB vector construct. 4-8 ZFP-KRAB designs were constructed for each gene target. Individual ZFP were evaluated for transient gene target repression in human leukemia cell line, K562. The best performing ZFP were moved from pER-GFP-KRAB vector to ZFPOFF_empty cloning vector. CD55 surface marker inhibition in K562 cells by ZFP-KRAB or ZFPOFF or CRISPRoff constructs is shown in FIG. 8A.

100,000 K562 cells were electroporated with 2 μg of plasmids of each construct (Lonza Nucleofector 4D, SF kit). For CRISPRoff comparison, 100,000 K562 cells were first electroporated with 2 μg vector carrying CRISPRoff dCas9 then 24 hrs after first electroporation, the same cells were electroporated with 3 μg sgRNA targeting CD55 promoter region (gRNA 5′-3′ sequence: GCUGCGACUCGGCGGAGUCC, Synthego; SEQ ID NO: 143). As shown in FIG. 8B, transfection efficiency was analyzed by gating % of cells expressing TagBFP marker (ZFPOFF_CD55_B4, ZFPOFF_empty, CRISPRoff) or eGFP marker (ZFPKRAB_B4).

Cell surface CD55 were stained with mouse anti-human CD55 PE antibody (Biolegend, Cat #311308) then analyzed by flow cytometry (Attune NxT, Thermofisher). Flow cytometry data were analyzed using Flowjo™ software with the same gating strategy for all samples. Graphs were plotted using GraphPad Prism. Data presented in representative of two biological replicates.

Cell surface staining protocol: cells were washed in stain buffer (BD Biosciences, Cat #554656). Cells were incubated on ice for 20 min in Human TruStain FcX™ (Fc Receptor Blocking Solution) diluted in stain buffer. After washing with stain buffer, cells were incubated on ice for 45 min in antibodies diluted in stain buffer. Cells were washed with PBS and stained in eBioscience™ Fixable Viability Dye eFluor™ 780 (Thermofisher, Cat #65-0865-14) or DAPI (Thermofisher, Cat #62248) to label dead cells.

Flow cytometry gating using Flowjo™: cells were gated for cell size by FSC and SSC. Single cells were gated by FSC-A and FSC-H. Live cells were gated by eFluor-780 negative or DAPI negative stain. Cell surface markers of interest were gated on either PE, APC or APC-Cy7 channels according to the antibodies used. As shown in FIG. 8B, ZFPKRAB plasmids in cells were mostly washed out after 9 days at which point the repression was no longer effective. In comparison, on-target repression induced by ZFPOFF was observed long after the plasmids were washed out. The long-term effect on gene repression by ZFPOFF and CRISPRoff V2.4 is comparable using the transient expression plasmid. Direct comparison between ZFPOFF and CRISPROFF mediated gene repression using transiently expression plasmid system in human leukemia cell line K562.

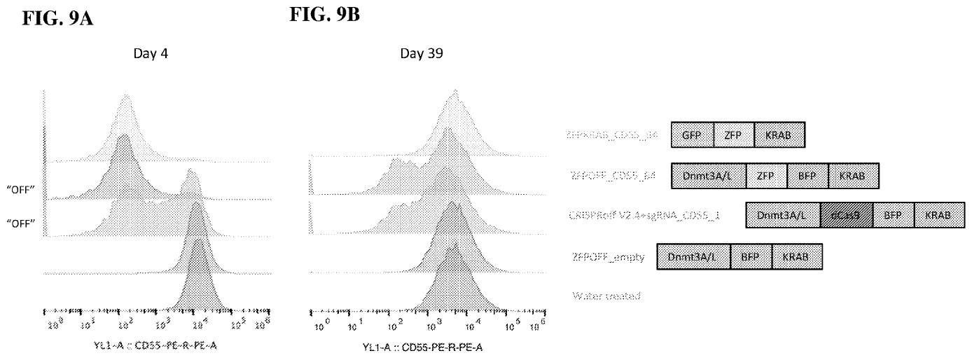

In addition, 100k K562 cells were electroporated as two biological replicates for ZFPOFF_CD55_B4, ZFPKRAB_CD55_B4 using the method described above. Cells were expanded and kept in tissue culture for 39 days after electroporation. As shown in FIG. 9A and FIG. 9B, long lasting repression of cell surface CD55 expression was observed by ZFPOFF and CRISPRoff but not ZFPKRAB.

### Example 3

mMESSAGE mMACHINE® T7 transcription kits (Thermofisher) were used for generating mRNA of ZFPoff and CRISPRoff constructs. In brief, pVAX plasmids containing T7 promoter and epigenome modulator were linearized with XhoI restriction enzyme then transcribed with ARCA 5′ capping, DNA template digestion with DNases, polyA tailing and subsequent lithium chloride precipitation according to manufacturer's recommendation.

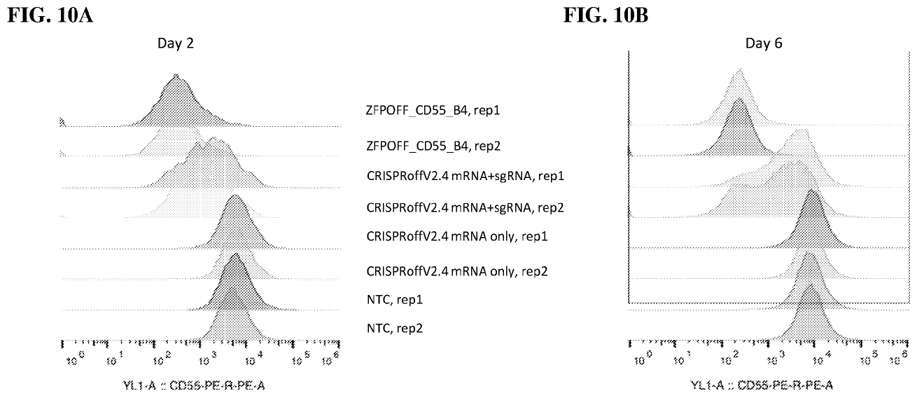

1.5 μg CRISPRoff mRNA and 1 μg of sgRNA targeting CD55 (CD55_1, GCUGCGACUCGGCGGAGUCC, Synthego; SEQ ID NO: 143) were co-delivered into 100,000 K562 cells by electroporation as described previously. 1.5 μg ZFPOFF CD55_B4 were delivered as a single electroporation reaction. Experiments were performed with biological duplicates. As shown in FIG. 10A and FIG. 10B, when delivered as mRNA in human leukemia cell line K562, ZFPOFF_CD55_B4 is more effective at repressing cell surface CD55 expression than co-delivery of CRISPRoff and sgRNA targeting CD55.

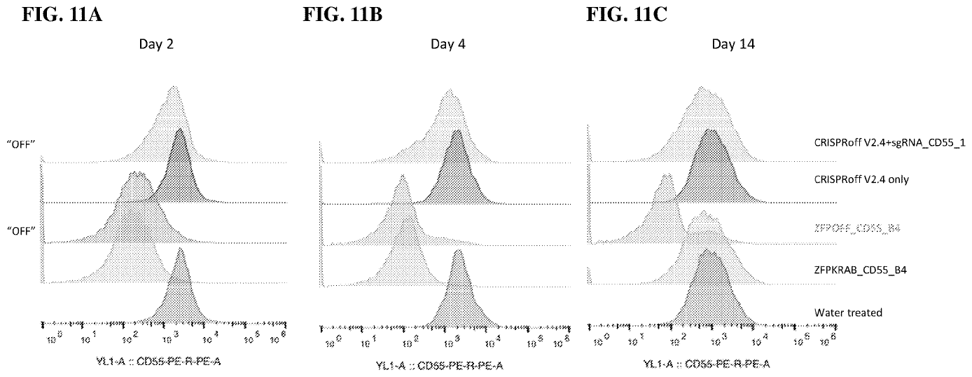

In addition, ZFP construct mRNA (4 μg) were delivered individually into 100,000 primary human HSPCs (STEMCELL Technologies) by electroporation (Lonza Nucleofector 4D, P3 kit). 4 μg CRISPRoff mRNA and sgRNA targeting CD55 were co-delivered at a ratio of (1:52 CRISPRoff to sgRNA ratio, respectively). Primary human HSPCs from a single donor were thawed and cultured in StemSpan™ Serum-Free Expansion Medium II (SFEM II) supplemented with CC110. Cells were electroporated with mRNA 48 hrs after thawing. As shown in FIG. 11A-11C, ZFPOFF_CD55_B4 mRNA delivery was effective at repressing cell surface CD55 expression in human primary peripheral blood HSPCs over a two-week period. CRISPRoff mRNA/sgRNA_CD55 co-delivery in PB HSPC was not effective.

### Example 4

ZFP were purchased from Sigma-Aldrich (Millipore) and delivered as plasmids on Sigma's pER-GFP-KRAB vector construct. 8 ZFP designs were constructed for each gene target. Individual ZFP were evaluated for transient gene target repression in human leukemia cell line, K562. The best performing 4 ZFPs per gene target were moved from pER-GFP-KRAB vector to ZFPOFF_empty cloning vector. 100,000 K562 cells were electroporated with 2 μg plasmids (Lonza Nucleofector 4D plus 96-well Shuttle, P3 kit) and cultured in one 96-well round bottom plate. Total RNA was extracted for qPCR analysis at 48 hr time point post electroporation (Direct-zol-96 RNA Kits, Zymo Research). 100 ng of RNA for each sample were converted to cDNA (High-Capacity cDNA Reverse Transcription Kit with RNase Inhibitor, Applied Biosystems™), qPCR was performed using pre-designed TaqMan probes and TaqMan™ Fast Advanced Master Mix (Thermofisher). Gene expression was normalized to GAPDH (ΔCT) then to empty_KRAB_only plasmid treated sample (ΔΔCT). Top 4 performing ZFP designs for each gene target were subsequently cloned into ZFPOFF_empty vector to couple validated ZFPs to epigenome modulators.

mRNA of ZFPOFFs targeting genes of interest were synthesized as described previously using mMESSAGE mMACHINE® T7 transcription kits (Thermofisher). 200,000 primary human HSPCs were electroporated with 4 μg individual ZFPOFF mRNA (Lonza Nucleofector 4D plus 96-well Shuttle, P3 kit) and cultured in one 96-well round bottom plate. 4 ZFPOFFs per gene target were tested without replicate in this initial HSPC screen. 4 days post electroporation, HSPC cultured in 96-well plate were split equally into two daughter plate (day 0 of irradiation). One plate was irradiated with 2-Gy radiation from a Cesium-137 source (Irr plate) while the other plate was not irradiated (Nirr). 2 days after irradiation (or day 6 post electroporation), samples of cells were collected for RNA extraction and qPCR to assess target gene expression level (target gene silencing). Cells were cultured and expanded in StemSpan™ Serum-Free Expansion Medium II (SFEM II) supplemented with CC110.

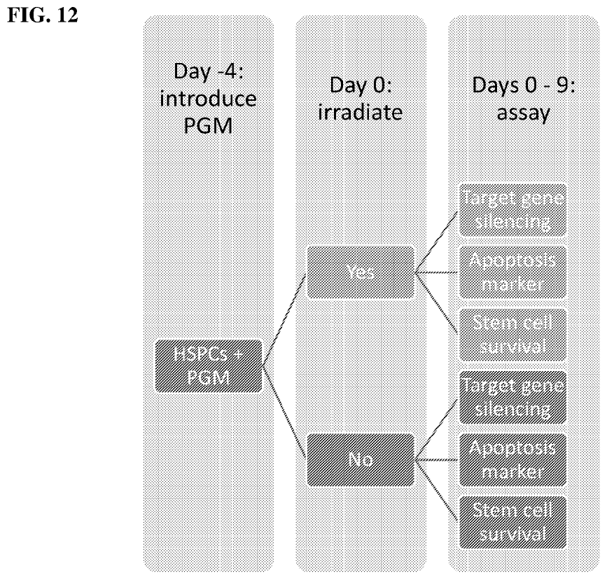

Apoptosis assays were performed on day 1, 3, 8 after irradiation from both 96-well plates (day 5, 8, 13 after electroporation) using apoptosis and dead cell staining kit for flow cytometry (Thermofisher, Cat #V13245). Apoptotic cells were labelled with Annexin V conjugated with Alexa Fluor 488 (Annexin-V-AF488). Dead cells were labelled with Propidium iodide (PI) according to manufacturer's protocols. The above-described method is depicted in FIG. 12.

Samples of cells from both plates were evaluated by cell surface marker staining for the content of lineage negative (Lin−), CD34+CD38lo, primitive hematopoietic stem and progenitor cells on day 2, 4, 9 after irradiation (or day 6, 10, 14 after electroporation). Antibodies: Hematopoietic Lineage Mouse anti-Human, APC, (eBioscience™, Cat #50-154-19), CD34 Mouse anti-Human, PE, Clone: 4H11, (eBioscience™, 50-103-33), APC/Cyanine7 anti-human CD38, clone HB-7, (Biolegend, Cat #356616).

Day 2 post irradiation (or day 6 post electroporation) HSPCs were collected for RNA extraction (Direct-zol-96 RNA Kits, Zymo Research). Normalized amount of RNA for each sample were converted to cDNA (High-Capacity cDNA Reverse Transcription Kit with RNase Inhibitor, Applied Biosystems™), qPCR was performed using pre-designed TaqMan probes and TaqMan™ Fast Advanced Master Mix (Thermofisher).

CT values of irradiated or non-irradiated samples were normalized to their corresponding (no ZFP) OFFctrl mRNA treated wells. Gene expression (CT) was normalized first to B2M CT value (ΔCT) then to empty_OFFctrl_only mRNA treated sample (ΔΔCT). 2^{-ΔΔCT} function converts normalization sample, OFFctrl values to 1 and experimental samples to relative expression level [Lin et al. Biostat Bioinforma Biomath. 2013 August; 3(3): 71-85.]. Error bars represent positive interval from 3 technical replicates. When OFFctrl mRNA were not available, CT values were normalized to CD55_B4_ZFPOFF treated wells instead (see CDKN1A ZFPOFF treatment samples).

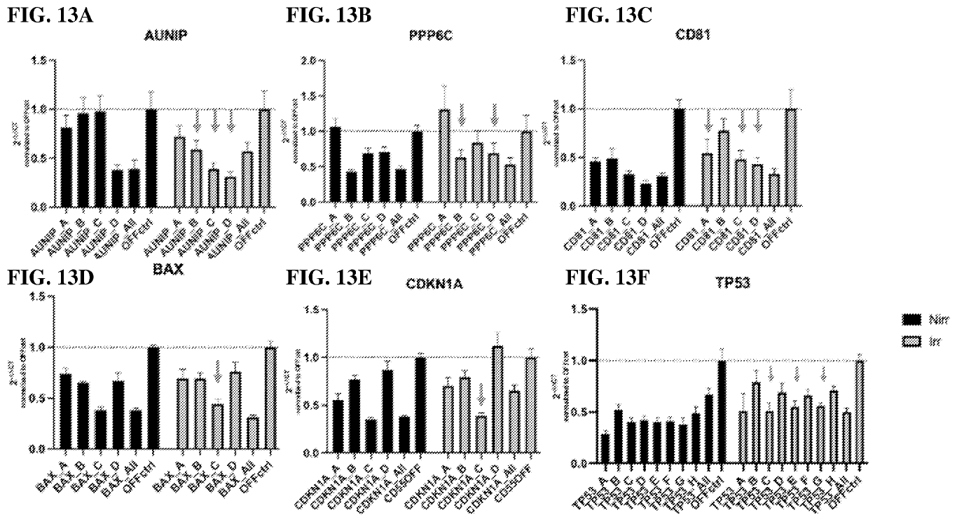

Irradiated or non-irradiated samples for each of AUNIP (FIG. 13A), PPP6C (FIG. 13B), CD81 (FIG. 13C), BAX (FIG. 13D), CDKN1A (FIG. 13E), and TP53 (FIG. 13F) gene targets were normalized to their corresponding (no ZFP) OFFctrl mRNA treated wells. Gene expression was normalized 1st to B2M (ΔCT) then to empty_OFFctrl_only mRNA treated sample (ΔΔCT). RNA was extracted from cells on Day 2 post irradiation (Day 6 after electroporation of ZFPOFF mRNA).

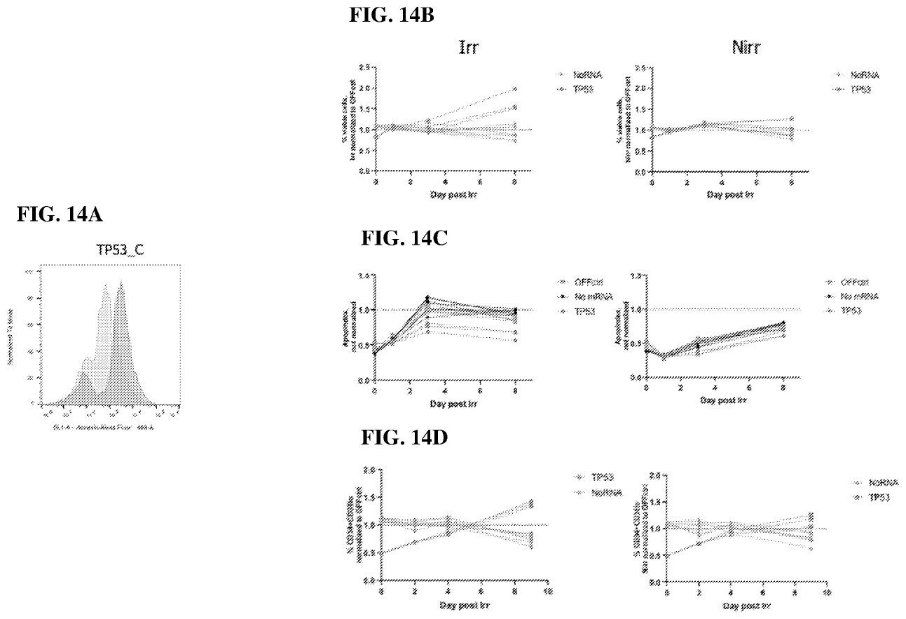

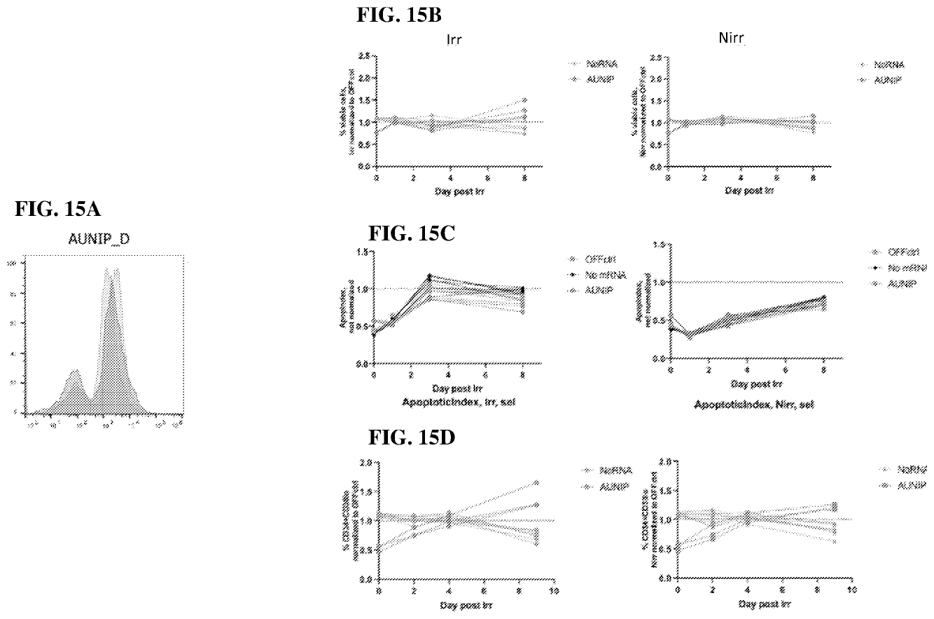

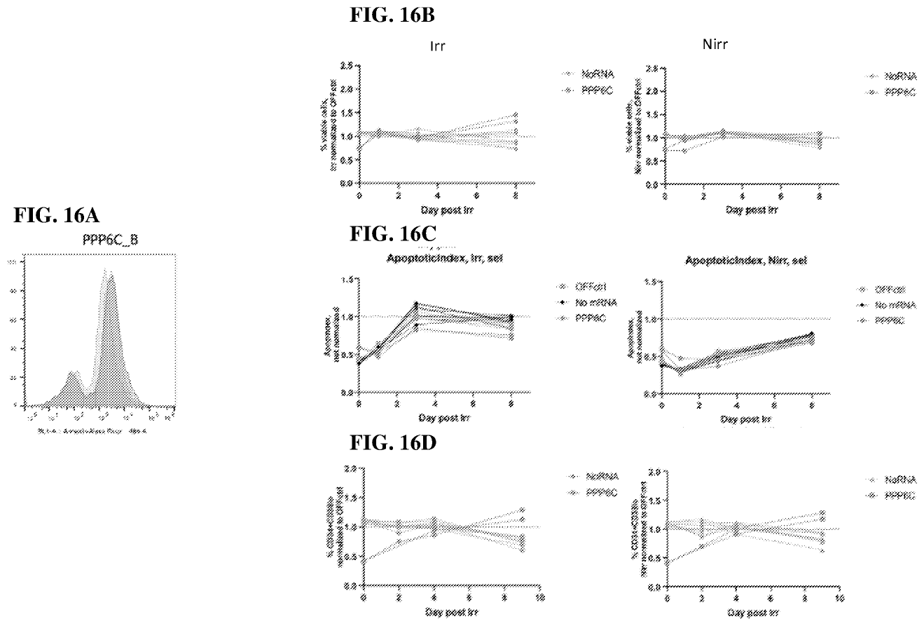

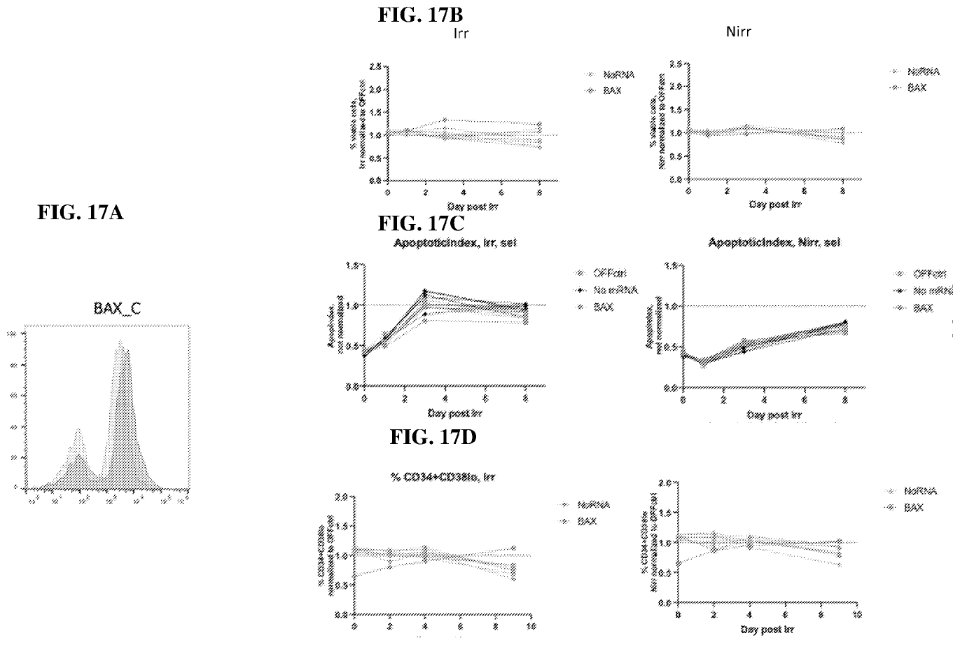

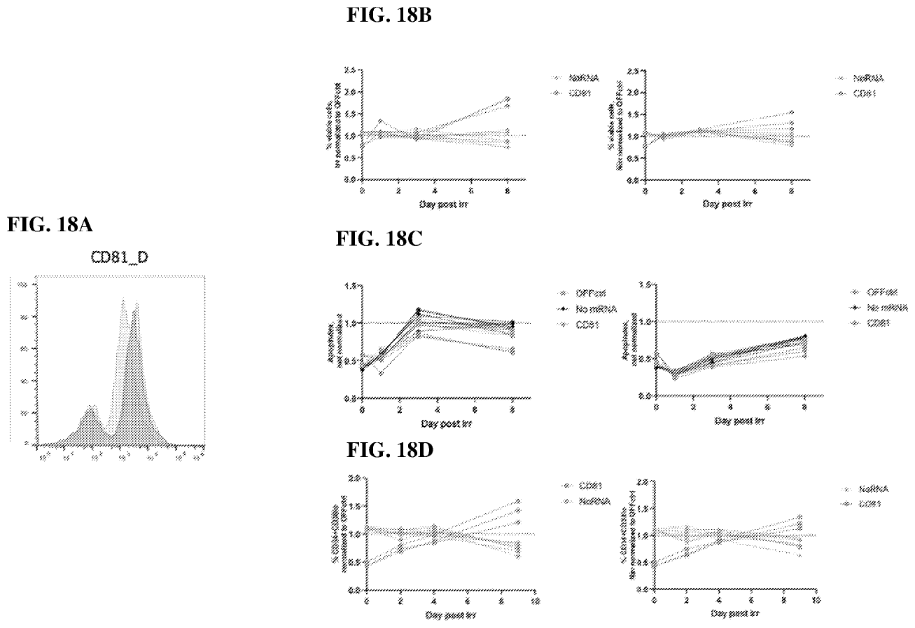

Apoptosis assays were performed on days 1, 3 and 8 after irradiation from both irradiated (lrr) and non-irradiated (Nirr) 96-well plates (day 5, 8, 13 after electroporation) using apoptosis and dead cell staining kit for flow cytometry (Thermofisher, Cat #V13245). Apoptotic cells were labelled with Annexin V conjugated with Alexa Fluor 488 (Annexin-V-AF488). Dead cells were labelled with Propidium iodide (PI) according to manufacturer's protocols. % viable cells were gated as PI negative, Annexin-V-AF488 negative single cells. Values from individual ZFPOFF treated wells were normalized to the average of 4 wells treated with OFFctrl only mRNA. FIG. 14B, FIG. 15B, FIG. 16B, FIG. 17B, and FIG. 18B depict a percentage of viable cells (Pl-, annexin V-) normalized to OFFctrl treated wells for TP53, AUNIP, PPP6C, BAX and CD81, respectively.

An “Apoptotic Index Value” (AIV) was generated by combining the fraction of apoptotic cells and geometric Mean Fluorescent Intensity (MFI) of Annexin-V-AF488 positive cells. Apoptotic index was calculated with the formula: (Annexin-V-AF488 positive fraction of single cells+Annexin-V-AF488 MFI)/10000. The higher the apoptotic index value, the more apoptotic cells exist in a given sample. This index was used to track apoptosis potential of irradiated samples. FIG. 14A and FIG. 14C, FIG. 15A and FIG. 15C, FIG. 16A and FIG. 16C, FIG. 17A and FIG. 17C, and FIG. 18A and FIG. 18C depict an Apoptotic Index generated by including annexin V MFI for TP53, AUNIP, PPP6C, BAX and CD81, respectively.

Samples of cells from both plates were evaluated by cell surface marker staining for the content of lineage negative (Lin−), CD34+CD38lo, primitive hematopoietic stem and progenitor cells on day 2, 4, 9 after irradiation (or day 6, 10, 14 after electroporation). Antibodies: Hematopoietic Lineage Mouse anti-Human, APC, (eBioscience™, Cat #50-154-19), CD34 Mouse anti-Human, PE, Clone: 4H11, (eBioscience™, 50-103-33), APC/Cyanine7 anti-human CD38, clone HB-7, (Biolegend, Cat #356616). Percentages of CD34+CD38lo HSPCs of individual ZFPOFF treated wells were normalized to that of the average of 4 wells treated with OFFctrl only mRNA. FIG. 14D, FIG. 15D, FIG. 16D, FIG. 17D, and FIG. 18D, show the promotion of stem cell growth via the repression of TP53, AUNIP, PPP6C, BAX and CD81, respectively. Based on the data from cell viability assays and stem cell marker staining, AUNIP, PPP6C, CD81 and BAX remain the top nominated gene targets for promoting HSPC growth and possible protection from apoptosis after irradiation.

### Example 5

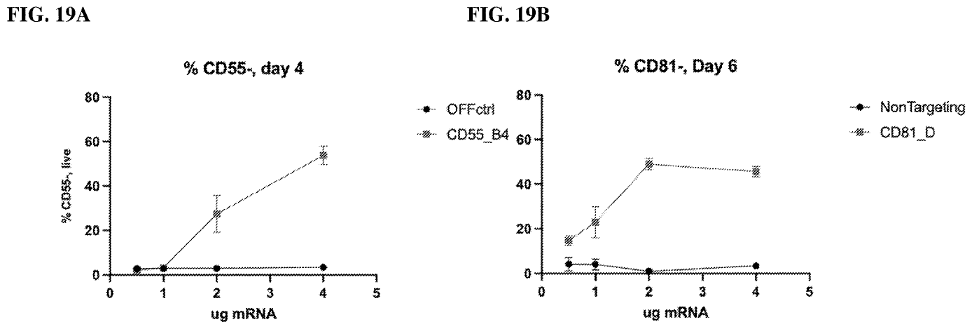

100,000 primary human HSPCs were electroporated with varying amounts of individual ZFPOFF mRNA targeting either CD55 (CD55_B4) or CD81 (CD81_D) (Lonza Nucleofector 4D plus 96-well Shuttle, P3 kit) and cultured in 96-well round bottom plate. OFFctrl ZFPOFF does not contain ZFP DNA-binding cassette. TP52_C ZFPOFF mRNA was used as a non-targeting control for CD81 surface staining. Experiment were performed as biological duplicates. CD55 cell surface staining and flow cytometry was performed on day 4 after mRNA electroporation. CD81 cell surface staining and flow cytometry was performed on day 6 after electroporation. Antibody for cell surface marker staining: PE anti-human CD55, clone JS11, Biolegend, Cat #311308. PE anti-human CD81 (TAPA-1), clone 5A6, Biolegend, Cat #349505. FIG. 19A-19B depict an optimization of ZFPoff mRNA dose using cell surface marker gene targets. As shown in FIG. 19A, increased target gene silencing was observed with increased dose. As shown in FIG. 19B, maximal on-target silencing was attained at intermediate dose.

### Example 6

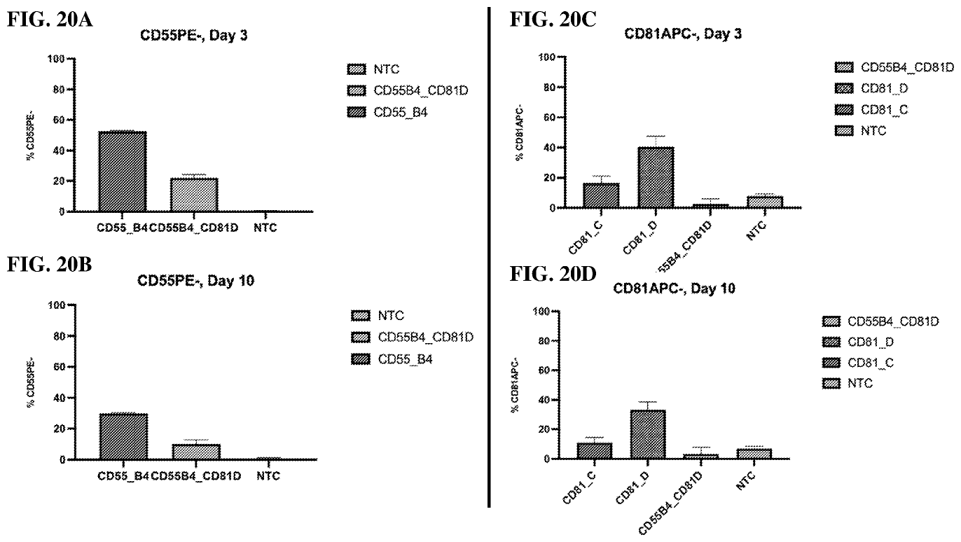

100,000 primary human HSPCs were electroporated with 2 μg individual ZFPOFF mRNA targeting either CD55 (CD55_B4) or CD81 (CD81_D) or with a combination of 1 μg of each CD55_B4 and CD81_D (Lonza Nucleofector 4D plus 96-well Shuttle, P3 kit) and cultured in 96-well round bottom plate. The experiment was performed with biological duplicates. CD55 and CD81 cell surface staining and flow cytometry was performed on day 3 and day 10 after mRNA electroporation. Antibody for cell surface marker staining: PE anti-human CD55, clone JS11, Biolegend, Cat #311308. APC anti-human CD81 (TAPA-1), clone 5A6, Biolegend, Cat #349510. Flow cytometry results for CD55 are shown in FIG. 20A-2B, while results for CD81 are shown in FIG. 20C-20D.

### Example 7

100,000 primary human HSPCs were electroporated with varying amounts of individual ZFPOFF mRNA targeting individually CD55 (CD55_B4) or CD81 (CD81_D), TP53 (TP53_C) or OFFctrl for no ZFP control (Lonza Nucleofector 4D plus 96-well Shuttle, P3 kit) and cultured in 96-well round bottom plate. Cells electroporated with 2 μl DEPC-treated nuclease-free water were used as ‘no mRNA input’ control. Cells were cultured and expanded in StemSpan™ Serum-Free Expansion Medium II (SFEM II) supplemented with CC110. The experiment was performed in biological duplicates.

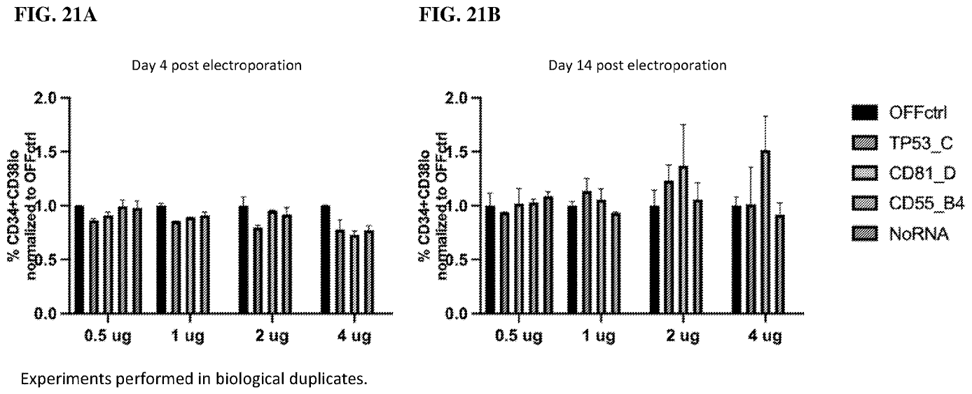

The impacts on primitive hematopoietic stem and progenitor cells among HSPCs were evaluated by lineage marker staining and flow cytometry on day 4 (FIG. 21A) and day 14 (FIG. 21B) after mRNA electroporation. Intermediate doses (2 μg) were well-tolerated at early and late timepoints. High doses (4 μg) modestly impair LT-HSC levels at early timepoints. Antibodies: Hematopoietic Lineage Mouse anti-Human, APC, (eBioscience™, Cat #50-154-19), CD34 Mouse anti-Human, PE, Clone: 4H11, (eBioscience™, 50-103-33), APC/Cyanine7 anti-human CD38, clone HB-7, (Biolegend, Cat #356616). % of CD34+CD38lo HSPCs of on-target ZFPOFF treated wells were normalized to that of the OFFctrl only mRNA treated wells.

### Example 8

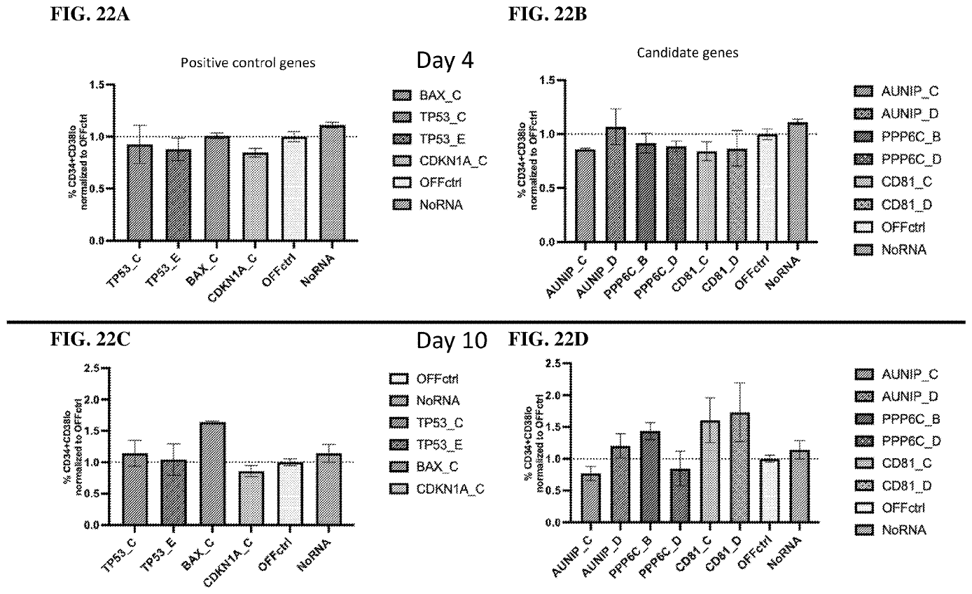

100,000 primary human HSPCs were electroporated with 2 μg of individual ZFPOFF mRNA targeting genes of interest. On day 4 and 10 after electroporation, % CD34+CD38lo primitive hematopoietic stem and progenitor cells among HSPCs were evaluated by lineage marker staining and flow cytometry as described previously. Experiments were performed in biological duplicates. On-target ZFPOFF treated wells were normalized to that of the OFFctrl only mRNA treated wells. As shown in FIG. 22A-22D, intermediate doses were well-tolerated and select PGMs promote survival at late timepoints.

### Example 9

Human primary T cells were activated with CD3/CD28 Dynabeads, 300 U/ml IL2, 5 ng/ml IL7, 5 ng/ml IL15 in X-Vivo15 media for 2 days. 3 days after activation, 100,000 activated T cells were electroporated with ZFPOFF mRNA targeting either CD55 or CD81 individually or in combination to demonstrate ZFPOFF multiplexing. For individual gene repression, 0.5, 1, 2 μg of the ZFPOFF mRNA were tested per 100,000 cells. For multiplexing (CD55+CD81 sample), 2 μg of each ZFPOFF mRNA target CD55 and CD81 (CD55_B4, CD81_C, respectively) were used with a combined mRNA amount of 4 μg. N=1.

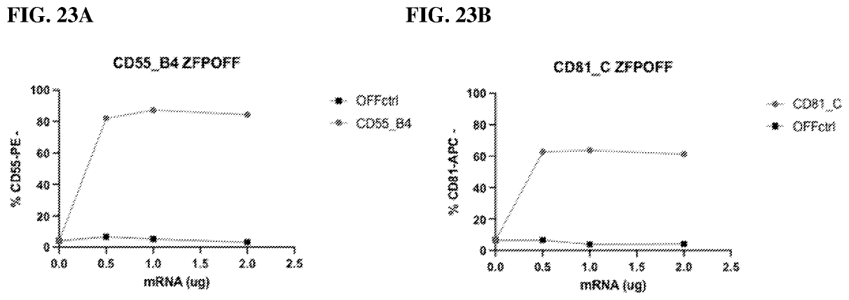

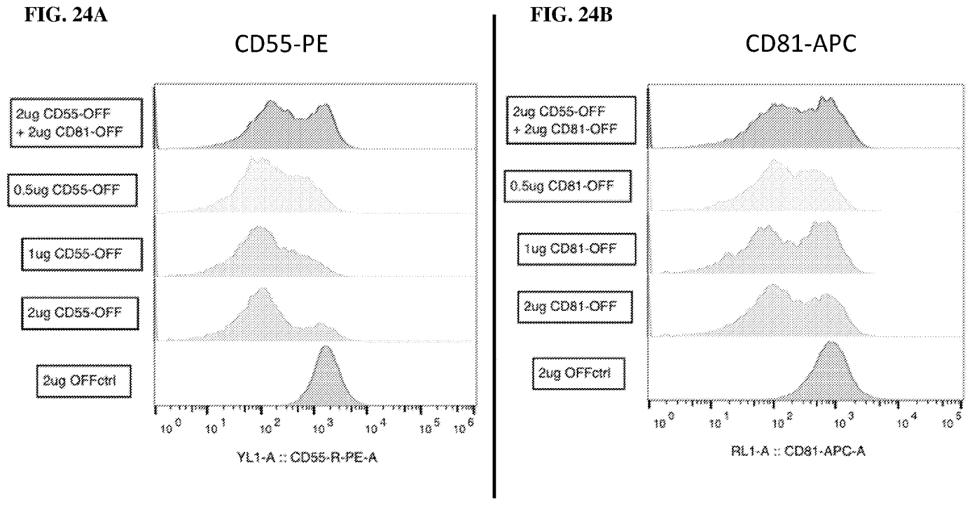

All electroporated T cells were stained for cell surface CD55 and CD81 using method described for K562 surface marker staining and flow cytometry. % of live cells negative for CD55 or CD81 markers were plotted in GraphPad Prism. Cell surface markers staining: all electroporated T cells were stained for cell surface CD55 and CD81 using method described for K562 surface marker staining and flow cytometry. % of live cells negative for CD55 or CD81 markers were plotted in GraphPad Prism. FIG. 23A-23B depict the optimization of ZFPoff mRNA dose using cell surface marker gene targets. As shown in FIG. 23A-23B, the lowest mRNA doses (0.5 μg) were able to achieve maximum gene repression for both CD55 and CD81 in activated T cells. A histogram of each treatment was normalized to maximum peak fluorescent intensity (Y-axis=% Maximum peak intensity; X-axis=fluorescence intensity) and overlayed with stagger offset (Flowjo™). These histograms are shown in FIG. 24A-24B.

### Example 10

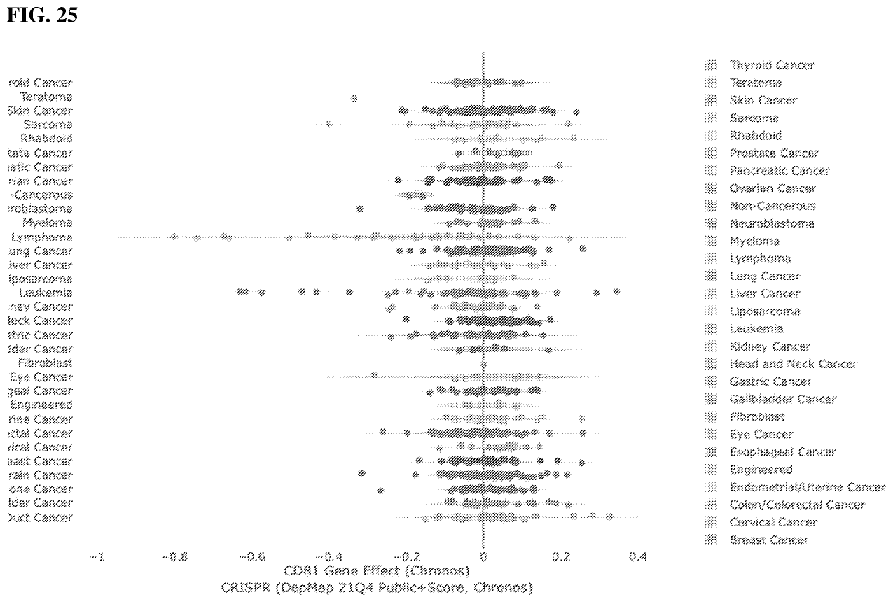

An analysis was carried out using DepMap Portal to show the sensitivity of certain cell lines to CD81 knockout. A resulting graph is shown in FIG. 25. As shown in FIG. 25, Some lymphoma and leukemia cell lines (but not K562) seemed to be more sensitive to CD81 KO.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A fusion protein comprising:
a zinc finger DNA binding polypeptide comprising an alpha-helix recognition domain configured to bind to a target nucleotide sequence in a target nucleic acid, wherein the target nucleic acid is selected from a target CD81 nucleic acid, a target BAX nucleic acid, a target PPP6C nucleic acid, and a target AUNIP nucleic acid; and
an epigenome modifying polypeptide.

2. The fusion protein according to claim 1, wherein the epigenome modifying polypeptide comprises a DNA methyltransferase domain.

3. The fusion protein according to claim 2, wherein the DNA methyltransferase polypeptide comprises a DNA methyltransferase 3 alpha (DNMT3A) domain.

4. The fusion protein according to claim 2, wherein the DNA methyltransferase polypeptide comprises a DNA methyltransferase 3 like (DNMT3L) domain.

5. The fusion protein according to any of claims 2 to 4, wherein the DNA methyltransferase polypeptide is a DNMT3A-3L polypeptide.

6. The fusion protein according to any of the preceding claims, wherein the fusion protein comprises, from N-terminus to C-terminus, the epigenome modifying polypeptide and the zinc finger DNA binding polypeptide.

7. The fusion protein according to any of the preceding claims, wherein the zinc finger DNA binding polypeptide and the epigenome modifying polypeptide are separated by a linker.

8. The fusion protein according to claim 7, wherein the linker is an XTEN linker.

9. The fusion protein according to claim 8, wherein the XTEN linker comprises 80 amino acids.

10. The fusion protein according to any of the preceding claims, further comprising a transcriptional repression polypeptide.

11. The fusion protein according to claim 10, wherein the transcriptional repression domain is a Kruppel associated box (KRAB) polypeptide.

12. The fusion protein according to claim 10 or 11, wherein the fusion protein comprises, from N-terminus to C-terminus, the epigenome modifying polypeptide, the zinc finger DNA binding polypeptide, and the transcriptional repression polypeptide.

13. The fusion protein according to any of claim 10 of 12, wherein the epigenome modifying polypeptide and the transcriptional repression polypeptide are separated by a linker.

14. The fusion protein according to claim 13, wherein the linker is an XTEN linker.

15. The fusion protein according to claim 14, wherein the XTEN linker comprises 16 amino acids.

16. The fusion protein according to any of the preceding claims, further comprising a nuclear localization signal (NLS).

17. A nucleic acid comprising a nucleotide sequence encoding the fusion protein of any one of claims 1-16.

18. The nucleic acid according to claim 17, wherein the nucleotide sequence is operably linked to a promoter.

19. The nucleic acid according to claim 18, wherein the promoter is functional in a hematopoietic stem cell (HSC) or a hematopoietic progenitor cell (HPC).

20. A recombinant expression vector comprising the nucleic acid of any of claims 17 to 19.

21. The recombinant expression vector according to claim 20, wherein the recombinant expression vector is a recombinant adenoassociated viral vector, a recombinant retroviral vector, or a recombinant lentiviral vector.

22. A hematopoietic stem cell (HSC) or a hematopoietic progenitor cell (HPC) comprising one or more of:
(a) the fusion protein according to any of claims 1 to 16;
(b) the nucleic acid according to any of claims 17 to 19; and
(c) the recombinant expression vector according to claim 20 or claim 21.

23. The HSC or HPC according to claim 22, wherein the HSC is a human HSC or wherein the HPC is a human HPC.

24. The HSC or HPC according to claim 22 or claim 23, wherein the nucleic acid is integrated into the genomic DNA of the HSC or the HPC.

25. A method of reducing ionizing radiation-induced damage to a hematopoietic stem cell (HSC) or a hematopoietic progenitor cell (HPC) in an individual, the method comprising introducing into the HSC or the HPC:
(a) the fusion protein according to any of claims 1 to 16;
(b) the nucleic acid according to any of claims 17 to 19; or
(c) the recombinant expression vector according to claim 20 or claim 21.

26. The method of claim 25, wherein the ionizing radiation is administered for the treatment of a cancer.

27. The method of claim 26, wherein the cancer is selected from small cell lung cancer, triple-negative breast cancer, human papilloma virus (HPV)-positive head and neck cancer, retinoblastoma, retinoblastoma protein (Rb)-negative bladder cancer, retinoblastoma protein (Rb)-negative prostate cancer, osteosarcoma, ovarian cancer, uterine cancer, and cervical cancer.

28. The method of claim 26 or claim 27, wherein the fusion protein, the nucleic acid, or the recombinant expression vector is administered from 1 hour to 48 hours before the ionizing radiation treatment for the cancer.

29. The method of claim 25, wherein the ionizing radiation occurs through an occupational, environmental, disaster, terrorist, or warfare event.

30. The method of any one of claims 25-29, comprising administering to the individual:
(a) the fusion protein according to any of claims 1 to 16;
(b) the nucleic acid according to any of claims 17 to 19; or
(c) the recombinant expression vector according to claim 20 or claim 21.


SEQUENCE LISTING (143 sequences)

SEQ ID NO: 1 - protein, 12 aa, synthetic construct
GVPSNLNPGKGS

SEQ ID NO: 2 - protein, 185 aa, synthetic construct
GVPAAMAERPFQCRICMRNFSRSDHLSEHIRTHTGEKPFACDICGRKFAARSTRTNHTKIHTGSQKPFQCRICMRNFSRSDDLTRHIRTHTGEKPFACDICGRKFAQSSDLRRHTKIHTGSQKPFQCRICMRNFSQSGSLTRHIRTHTGEKPFACDICGRKFADRSHLARHTKIHLRQKDAARGS

SEQ ID NO: 3 - protein, 977 aa, synthetic construct
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVGVPSNLNPGKGSPKKKRKVSGSETPGTSESATPESSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNGGGGGMDAKSLTAWSRSRTLVTFKDVFVDFTREEWKLLDTAQQIVYRNVMLENYKNLVSLGYQLTKPDVILRLEKGEEP

SEQ ID NO: 4 - protein, 1150 aa, synthetic construct
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVGVPAAMAERPFQCRICMRNFSRSDHLSEHIRTHTGEKPFACDICGRKFAARSTRTNHTKIHTGSQKPFQCRICMRNFSRSDDLTRHIRTHTGEKPFACDICGRKFAQSSDLRRHTKIHTGSQKPFQCRICMRNFSQSGSLTRHIRTHTGEKPFACDICGRKFADRSHLARHTKIHLRQKDAARGSPKKKRKVSGSETPGTSESATPESSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNGGGGGMDAKSLTAWSRSRTLVTFKDVFVDFTREEWKLLDTAQQIVYRNVMLENYKNLVSLGYQLTKPDVILRLEKGEEP

SEQ ID NO: 5 - DNA, 2934 bp, synthetic construct
  source 1..2934: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccgcatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggcacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaggggtaccgtcaaatttaaatcccgggaagggatcccccaagaagaagaggaaggtgagcggaagtgagaccccaggtacatccgaatcagcaacgcctgaaagcagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggcggtggcggagggatggatgctaagtcactaactgcctggtccagatctcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctag

SEQ ID NO: 6 - DNA, 3453 bp, synthetic construct
  source 1..3453: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccgcatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggcacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaggggtacccgccgctatggctgagaggcccttccagtgtcgaatctgcatgcgtaacttcagtcgctccgaccacctgtccgagcacatccgcacccacaccggcgagaagccttttgcctgtgacatttgtgggaggaaatttgccgcccgctccacccgcaccaaccataccaagatacacacgggcagccaaaagcccttccagtgtcgaatctgcatgcgtaacttcagtcgctccgacgacctgacccgccacatccgcacccacaccggcgagaagccttttgcctgtgacatttgtgggaggaaatttgcccagtcctccgacctgcgccgccataccaagatacacacgggatctcagaagcccttccagtgtcgaatctgcatgcgtaacttcagtcagtccggctccctgacccgccacatccgcacccacaccggcgagaagccttttgcctgtgacatttgtgggaggaaatttgccgaccgctcccacctggcccgccataccaagatacacctgcgccaaaaagatgcggcccggggatcccccaagaagaagaggaaggtgagcggaagtgagaccccaggtacatccgaatcagcaacgcctgaaagcagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggcggtggcggagggatggatgctaagtcactaactgcctggtccagatctcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctag

SEQ ID NO: 7 - DNA, 5900 bp, synthetic construct
  source 1..5900: mol_type = other DNA
gactcttcgcgatgtacgggccagatatacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagctgatccactagtccagtgtggtggaattcgccaccatgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccgcatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggcacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaggggtaccgtcaaatttaaatcccgggaagggatcccccaagaagaagaggaaggtgagcggaagtgagaccccaggtacatccgaatcagcaacgcctgaaagcagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggcggtggcggagggatggatgctaagtcactaactgcctggtccagatctcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctagtaaagatcttgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 8-10 - skipped (3 entries)

SEQ ID NO: 11 - protein, 185 aa, synthetic construct
GVPAAMAERPFQCRICMRNFSDDWNLSQHIRTHTGEKPFACDICGRKFARSANLTRHTKIHTGSQKPFQCRICMRNFSHKQHRDAHIRTHTGEKPFACDICGRKFARSANLTRHTKIHTGSQKPFQCRICMRNFSRSDDLTRHIRTHTGEKPFACDICGRKFAMSYQLGRHTKIHLRQKDAARGS

SEQ ID NO: 12 - protein, 185 aa, synthetic construct
GVPAAMAERPFQCRICMRNFSDRSDLSRHIRTHTGEKPFACDICGRKFARRTDLRRHTKIHTGSQKPFQCRICMRNFSRSDDLTRHIRTHTGEKPFACDICGRKFADRSTLARHTKIHTGSQKPFQCRICMRNFSRSAHLSRHIRTHTGEKPFACDICGRKFADRSHLARHTKIHLRQKDAARGS

SEQ ID NO: 13 - protein, 185 aa, synthetic construct
GVPAAMAERPFQCRICMRNFSRSADLTRHIRTHTGEKPFACDICGRKFADRSHLARHTKIHTGSQKPFQCRICMRNFSYKCCLSDHIRTHTGEKPFACDICGRKFARSANLTRHTKIHTGSQKPFQCRICMRNFSRSDTLSQHIRTHTGEKPFACDICGRKFVARSTRITHTKIHLRQKDAARGS

SEQ ID NO: 14 - protein, 185 aa, synthetic construct
GVPAAMAERPFQCRICMRNFSQSSDLSRHIRTHTGEKPFACDICGRKFADRSVLRRHTKIHTGSQKPFQCRICMRNFSDNRDLSTHIRTHTGEKPFACDICGRKFADRSNLTRHTKIHTGSQKPFQCRICMRNFSDRSHLSRHIRTHTGEKPFACDICGRKFADRSHLARHTKIHLRQKDAARGS

SEQ ID NO: 15 - protein, 185 aa, synthetic construct
GVPAAMAERPFQCRICMRNFSDRSHLTRHIRTHTGEKPFACDICGRKFARSDHLSRHTKIHTGSQKPFQCRICMRNFSQSGNLARHIRTHTGEKPFACDICGRKFAATCCLAHHTKIHTGSQKPFQCRICMRNFSDQSNLRAHIRTHTGEKPFACDICGRKFARSAHLSRHTKIHLRQKDAARGS

SEQ ID NO: 16 - protein, 185 aa, synthetic construct
GVPAAMAERPFQCRICMRNFSRSDDLTRHIRTHTGEKPFACDICGRKFAMSYQLGRHTKIHTGSQKPFQCRICMRNFSDRSDLSRHIRTHTGEKPFACDICGRKFARRTDLRRHTKIHTGSQKPFQCRICMRNFSRSDVLSEHIRTHTGEKPFACDICGRKFARSAHLSRHTKIHLRQKDAARGS

SEQ ID NO: 17 - protein, 185 aa, synthetic construct
GVPAAMAERPFQCRICMRNFSQSSNLARHIRTHTGEKPFACDICGRKFADSSDRKKHTKIHTGSQKPFQCRICMRNFSHHCCLTAHIRTHTGEKPFACDICGRKFARSADLSRHTKIHTGSQKPFQCRICMRNFSDRSHLTRHIRTHTGEKPFACDICGRKFAHRSARKRHTKIHLRQKDAARGS

SEQ ID NO: 18 - protein, 185 aa, synthetic construct
GVPAAMAERPFQCRICMRNFSRSHSLLRHIRTHTGEKPFACDICGRKFARLDWLPMHTKIHTGSQKPFQCRICMRNFSTSGSLSRHIRTHTGEKPFACDICGRKFARSDDLTRHTKIHTGSQKPFQCRICMRNFSERGTLARHIRTHTGEKPFACDICGRKFARSDDLTRHTKIHLRQKDAARGS

SEQ ID NO: 19 - protein, 185 aa, synthetic construct
GVPAAMAERPFQCRICMRNFSQNAHRKTHIRTHTGEKPFACDICGRKFARSAHLSRHTKIHTGSQKPFQCRICMRNFSRSDDLTRHIRTHTGEKPFACDICGRKFAQSGSLTRHTKIHTGSQKPFQCRICMRNFSDRSNLSRHIRTHTGEKPFACDICGRKFADRTSLRYHTKIHLRQKDAARGS

SEQ ID NO: 20 - protein, 185 aa, synthetic construct
GVPAAMAERPFQCRICMRNFSQSGDLTRHIRTHTGEKPFACDICGRKFARRADLSRHTKIHTGSQKPFQCRICMRNFSRSDNLSEHIRTHTGEKPFACDICGRKFARSADLSRHTKIHTGSQKPFQCRICMRNFSQSSDLSRHIRTHTGEKPFACDICGRKFADRSHLARHTKIHLRQKDAARGS

SEQ ID NO: 21 - protein, 185 aa, synthetic construct
GVPAAMAERPFQCRICMRNFSDSSDRKKHIRTHTGEKPFACDICGRKFARRSDLKRHTKIHTGSQKPFQCRICMRNFSQSSDLSRHIRTHTGEKPFACDICGRKFARRDTLLDHTKIHTGSQKPFQCRICMRNFSTSGSLSRHIRTHTGEKPFACDICGRKFAQAGQRRVHTKIHLRQKDAARGS

SEQ ID NO: 22 - protein, 185 aa, synthetic construct
GVPAAMAERPFQCRICMRNFSQQSDLRVHIRTHTGEKPFACDICGRKFARSADLSRHTKIHTGSQKPFQCRICMRNFSRSDSLLRHIRTHTGEKPFACDICGRKFAQSVRLPHHTKIHTGSQKPFQCRICMRNFSRKQTRTTHIRTHTGEKPFACDICGRKFADSSHRTRHTKIHLRQKDAARGS

SEQ ID NO: 23-34 - skipped (12 entries)

SEQ ID NO: 35 - protein, 185 aa, synthetic construct
GVPAAMAERPFQCRICMRNFSRSDHLSEHIRTHTGEKPFACDICGRKFARMYTLSKHTKIHTGSQKPFQCRICMRNFSRSDNLSTHIRTHTGEKPFACDICGRKFARSTHRTQHTKIHTGSQKPFQCRICMRNFSRSADLTRHIRTHTGEKPFACDICGRKFAQSGHLSRHTKIHLRQKDAARGS

SEQ ID NO: 36 - protein, 185 aa, synthetic construct
GVPAAMAERPFQCRICMRNFSSEADRSRHIRTHTGEKPFACDICGRKFADRSNLTRHTKIHTGSQKPFQCRICMRNFSRSDNLSVHIRTHTGEKPFACDICGRKFARRFLRNNHTKIHTGSQKPFQCRICMRNFSYDYGRYTHIRTHTGEKPFACDICGRKFADRSHLARHTKIHLRQKDAARGS

SEQ ID NO: 37 - protein, 185 aa, synthetic construct
GVPAAMAERPFQCRICMRNFSDRSHLTRHIRTHTGEKPFACDICGRKFAQSAHLKAHTKIHTGSQKPFQCRICMRNFSRSHSLLRHIRTHTGEKPFACDICGRKFARRDTLLDHTKIHTGSQKPFQCRICMRNFSDRSNLSRHIRTHTGEKPFACDICGRKFADAFTRTRHTKIHLRQKDAARGS

SEQ ID NO: 38 - protein, 185 aa, synthetic construct
GVPAAMAERPFQCRICMRNFSRSDAMSQHIRTHTGEKPFACDICGRKFARNASRTRHTKIHTGSQKPFQCRICMRNFSRSADLTRHIRTHTGEKPFACDICGRKFARSDDLTRHTKIHTGSQKPFQCRICMRNFSHACCRNMHIRTHTGEKPFACDICGRKFARSADLSRHTKIHLRQKDAARGS

SEQ ID NO: 39-54 - skipped (16 entries)

SEQ ID NO: 55 - DNA, 36 bp, synthetic construct
  source 1..36: mol_type = other DNA
ggggtaccgtcaaatttaaatcccgggaagggatcc

SEQ ID NO: 56 - DNA, 555 bp, synthetic construct
  source 1..555: mol_type = other DNA
ggggtacccgccgctatggctgagaggcccttccagtgtcgaatctgcatgcgtaacttcagtcgctccgaccacctgtccgagcacatccgcacccacaccggcgagaagccttttgcctgtgacatttgtgggaggaaatttgccgcccgctccacccgcaccaaccataccaagatacacacgggcagccaaaagcccttccagtgtcgaatctgcatgcgtaacttcagtcgctccgacgacctgacccgccacatccgcacccacaccggcgagaagccttttgcctgtgacatttgtgggaggaaatttgcccagtcctccgacctgcgccgccataccaagatacacacgggatctcagaagcccttccagtgtcgaatctgcatgcgtaacttcagtcagtccggctccctgacccgccacatccgcacccacaccggcgagaagccttttgcctgtgacatttgtgggaggaaatttgccgaccgctcccacctggcccgccataccaagatacacctgcgccaaaaagatgcggcccggggatcc

SEQ ID NO: 57-64 - skipped (8 entries)

SEQ ID NO: 65 - DNA, 555 bp, synthetic construct
  source 1..555: mol_type = other DNA
ggggtacccgccgctatggctgagaggcccttccagtgtcgaatctgcatgcgtaacttcagtgacgactggaacctgtcccagcacatccgcacccacaccggcgagaagccttttgcctgtgacatttgtgggaggaaatttgcccgctccgccaacctgacccgccataccaagatacacacgggcagccaaaagcccttccagtgtcgaatctgcatgcgtaacttcagtcacaagcagcaccgcgacgcccacatccgcacccacaccggcgagaagccttttgcctgtgacatttgtgggaggaaatttgcccgctccgccaacctgacccgccataccaagatacacacgggatctcagaagcccttccagtgtcgaatctgcatgcgtaacttcagtcgctccgacgacctgacccgccacatccgcacccacaccggcgagaagccttttgcctgtgacatttgtgggaggaaatttgccatgtcctaccagctgggccgccataccaagatacacctgcgccaaaaagatgcggcccggggatcc

SEQ ID NO: 66 - DNA, 555 bp, synthetic construct
  source 1..555: mol_type = other DNA
ggggtacccgccgctatggctgagaggcccttccagtgtcgaatctgcatgcgtaacttcagtgaccgctccgacctgtcccgccacatccgcacccacaccggcgagaagccttttgcctgtgacatttgtgggaggaaatttgcccgccgcaccgacctgcgccgccataccaagatacacacgggcagccaaaagcccttccagtgtcgaatctgcatgcgtaacttcagtcgctccgacgacctgacccgccacatccgcacccacaccggcgagaagccttttgcctgtgacatttgtgggaggaaatttgccgaccgctccaccctggcccgccataccaagatacacacgggatctcagaagcccttccagtgtcgaatctgcatgcgtaacttcagtcgctccgcccacctgtcccgccacatccgcacccacaccggcgagaagccttttgcctgtgacatttgtgggaggaaatttgccgaccgctcccacctggcccgccataccaagatacacctgcgccaaaaagatgcggcccggggatcc

SEQ ID NO: 67 - DNA, 555 bp, synthetic construct
  source 1..555: mol_type = other DNA
ggggtacccgccgctatggctgagaggcccttccagtgtcgaatctgcatgcgtaacttcagtcgctccgccgacctgacccgccacatccgcacccacaccggcgagaagccttttgcctgtgacatttgtgggaggaaatttgccgaccgctcccacctggcccgccataccaagatacacacgggcagccaaaagcccttccagtgtcgaatctgcatgcgtaacttcagttacaagtgttgcctgtccgaccacatccgcacccacaccggcgagaagccttttgcctgtgacatttgtgggaggaaatttgcccgctccgccaacctgacccgccataccaagatacacacgggatctcagaagcccttccagtgtcgaatctgcatgcgtaacttcagtcgtagtgacaccctgagccagcacatccgcacccacacaggcgagaagccttttgcctgtgacatttgtgggagaaaatttgtcgccaggagcacccgcataacccataccaagatacacctgcgccaaaaagatgcggcccggggatcc

SEQ ID NO: 68 - DNA, 555 bp, synthetic construct
  source 1..555: mol_type = other DNA
ggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccgaccgctccgtgctgcgccgccataccaagatacacacgggcagccaaaagcccttccagtgtcgaatctgcatgcgtaacttcagtgacaaccgcgacctgtccacccacatccgcacccacaccggcgagaagccttttgcctgtgacatttgtgggaggaaatttgccgaccgctccaacctgacccgccataccaagatacacacgggatctcagaagcccttccagtgtcgaatctgcatgcgtaacttcagtgaccgctcccacctgtcccgccacatccgcacccacaccggcgagaagccttttgcctgtgacatttgtgggaggaaatttgccgaccgctcccacctggcccgccataccaagatacacctgcgccaaaaagatgcggcccggggatcc

SEQ ID NO: 69 - DNA, 555 bp, synthetic construct
  source 1..555: mol_type = other DNA
ggggtacccgccgctatggctgagaggcccttccagtgtcgaatctgcatgcgtaacttcagtgaccgctcccacctgacccgccacatccgcacccacaccggcgagaagccttttgcctgtgacatttgtgggaggaaatttgcccgctccgaccacctgtcccgccataccaagatacacacgggcagccaaaagcccttccagtgtcgaatctgcatgcgtaacttcagtcagtccggcaacctggcccgccacatccgcacccacaccggcgagaagccttttgcctgtgacatttgtgggaggaaatttgccgccacctgttgtctggcccaccataccaagatacacacgggatctcagaagcccttccagtgtcgaatctgcatgcgtaacttcagtgaccagtccaacctgcgcgcccacatccgcacccacaccggcgagaagccttttgcctgtgacatttgtgggaggaaatttgcccgctccgcccacctgtcccgccataccaagatacacctgcgccaaaaagatgcggcccggggatcc

SEQ ID NO: 70 - DNA, 555 bp, synthetic construct
  source 1..555: mol_type = other DNA
ggggtacccgccgctatggctgagaggcccttccagtgtcgaatctgcatgcgtaacttcagtcgctccgacgacctgacccgccacatccgcacccacaccggcgagaagccttttgcctgtgacatttgtgggaggaaatttgccatgtcctaccagctgggccgccataccaagatacacacgggcagccaaaagcccttccagtgtcgaatctgcatgcgtaacttcagtgaccgctccgacctgtcccgccacatccgcacccacaccggcgagaagccttttgcctgtgacatttgtgggaggaaatttgcccgccgcaccgacctgcgccgccataccaagatacacacgggatctcagaagcccttccagtgtcgaatctgcatgcgtaacttcagtcgctccgacgtgctgtccgagcacatccgcacccacaccggcgagaagccttttgcctgtgacatttgtgggaggaaatttgcccgctccgcccacctgtcccgccataccaagatacacctgcgccaaaaagatgcggcccggggatcc

SEQ ID NO: 71 - DNA, 555 bp, synthetic construct
  source 1..555: mol_type = other DNA
ggggtacccgccgctatggctgagaggcccttccagtgtcgaatctgcatgcgtaacttcagtcagtcctccaacctggcccgccacatccgcacccacaccggcgagaagccttttgcctgtgacatttgtgggaggaaatttgccgactcctccgaccgcaagaagcataccaagatacacacgggcagccaaaagcccttccagtgtcgaatctgcatgcgtaacttcagtcaccactgttgtctgaccgcccacatccgcacccacaccggcgagaagccttttgcctgtgacatttgtgggaggaaatttgcccgctccgccgacctgtcccgccataccaagatacacacgggatctcagaagcccttccagtgtcgaatctgcatgcgtaacttcagtgaccgctcccacctgacccgccacatccgcacccacaccggcgagaagccttttgcctgtgacatttgtgggaggaaatttgcccaccgctccgcccgcaagcgccataccaagatacacctgcgccaaaaagatgcggcccggggatcc

SEQ ID NO: 72 - DNA, 555 bp, synthetic construct
  source 1..555: mol_type = other DNA
ggggtacccgccgctatggctgagaggcccttccagtgtcgaatctgcatgcgtaacttcagtcgctcccactccctgctgcgccacatccgcacccacaccggcgagaagccttttgcctgtgacatttgtgggaggaaatttgcccgcctggactggctgccgatgcataccaagatacacacgggcagccaaaagcccttccagtgtcgaatctgcatgcgtaacttcagtacctccggctccctgtcccgccacatccgcacccacaccggcgagaagccttttgcctgtgacatttgtgggaggaaatttgcccgctccgacgacctgacccgccataccaagatacacacgggatctcagaagcccttccagtgtcgaatctgcatgcgtaacttcagtgagcgcggcaccctggcccgccacatccgcacccacaccggcgagaagccttttgcctgtgacatttgtgggaggaaatttgcccgctccgacgacctgacccgccataccaagatacacctgcgccaaaaagatgcggcccggggatcc

SEQ ID NO: 73 - DNA, 555 bp, synthetic construct
  source 1..555: mol_type = other DNA
ggggtacccgccgctatggctgagaggcccttccagtgtcgaatctgcatgcgtaacttcagtcagaacgcccaccgcaagacccacatccgcacccacaccggcgagaagccttttgcctgtgacatttgtgggaggaaatttgcccgctccgcccacctgtcccgccataccaagatacacacgggcagccaaaagcccttccagtgtcgaatctgcatgcgtaacttcagtcgctccgacgacctgacccgccacatccgcacccacaccggcgagaagccttttgcctgtgacatttgtgggaggaaatttgcccagtccggctccctgacccgccataccaagatacacacgggatctcagaagcccttccagtgtcgaatctgcatgcgtaacttcagtgaccgctccaacctgtcccgccacatccgcacccacaccggcgagaagccttttgcctgtgacatttgtgggaggaaatttgccgaccgcacctccctgcgctaccataccaagatacacctgcgccaaaaagatgcggcccggggatcc

SEQ ID NO: 74 - DNA, 555 bp, synthetic construct
  source 1..555: mol_type = other DNA
ggggtacccgccgctatggctgagaggcccttccagtgtcgaatctgcatgcgtaacttcagtcagtccggcgacctgacccgccacatccgcacccacaccggcgagaagccttttgcctgtgacatttgtgggaggaaatttgcccgccgcgccgacctgtcccgccataccaagatacacacgggcagccaaaagcccttccagtgtcgaatctgcatgcgtaacttcagtcgctccgacaacctgtccgagcacatccgcacccacaccggcgagaagccttttgcctgtgacatttgtgggaggaaatttgcccgctccgccgacctgtcccgccataccaagatacacacgggatctcagaagcccttccagtgtcgaatctgcatgcgtaacttcagtcagtcctccgacctgtcccgccacatccgcacccacaccggcgagaagccttttgcctgtgacatttgtgggaggaaatttgccgaccgctcccacctggcccgccataccaagatacacctgcgccaaaaagatgcggcccggggatcc

SEQ ID NO: 75 - DNA, 555 bp, synthetic construct
  source 1..555: mol_type = other DNA
ggggtacccgccgctatggctgagaggcccttccagtgtcgaatctgcatgcgtaacttcagtgactcctccgaccgcaagaagcacatccgcacccacaccggcgagaagccttttgcctgtgacatttgtgggaggaaatttgcccgccgctccgacctgaagcgccataccaagatacacacgggcagccaaaagcccttccagtgtcgaatctgcatgcgtaacttcagtcagtcctccgacctgtcccgccacatccgcacccacaccggcgagaagccttttgcctgtgacatttgtgggaggaaatttgcccgccgcgacaccctgctggaccataccaagatacacacgggatctcagaagcccttccagtgtcgaatctgcatgcgtaacttcagtacctccggctccctgtcccgccacatccgcacccacaccggcgagaagccttttgcctgtgacatttgtgggaggaaatttgcccaggccggccagcgccgcgtgcataccaagatacacctgcgccaaaaagatgcggcccggggatcc

SEQ ID NO: 76 - DNA, 555 bp, synthetic construct
  source 1..555: mol_type = other DNA
ggggtacccgccgctatggctgagaggcccttccagtgtcgaatctgcatgcgtaacttcagtcagcagtccgacctgcgcgtgcacatccgcacccacaccggcgagaagccttttgcctgtgacatttgtgggaggaaatttgcccgctccgccgacctgtcccgccataccaagatacacacgggcagccaaaagcccttccagtgtcgaatctgcatgcgtaacttcagtcgctccgactccctgctgcgccacatccgcacccacaccggcgagaagccttttgcctgtgacatttgtgggaggaaatttgcccagtccgtgcgcctgccgcaccataccaagatacacacgggatctcagaagcccttccagtgtcgaatctgcatgcgtaacttcagtcgcaagcagacccgcaccacccacatccgcacccacaccggcgagaagccttttgcctgtgacatttgtgggaggaaatttgccgactcctcccaccgcacccgccataccaagatacacctgcgccaaaaagatgcggcccggggatcc

SEQ ID NO: 77-88 - skipped (12 entries)

SEQ ID NO: 89 - DNA, 555 bp, synthetic construct
  source 1..555: mol_type = other DNA
ggggtacccgccgctatggctgagaggcccttccagtgtcgaatctgcatgcgtaacttcagtcgctccgaccacctgtccgagcacatccgcacccacaccggcgagaagccttttgcctgtgacatttgtgggaggaaatttgcccgcatgtacaccctgtccaagcataccaagatacacacgggcagccaaaagcccttccagtgtcgaatctgcatgcgtaacttcagtcgctccgacaacctgtccacccacatccgcacccacaccggcgagaagccttttgcctgtgacatttgtgggaggaaatttgcccgctccacccaccgcacccagcataccaagatacacacgggatctcagaagcccttccagtgtcgaatctgcatgcgtaacttcagtcgctccgccgacctgacccgccacatccgcacccacaccggcgagaagccttttgcctgtgacatttgtgggaggaaatttgcccagtccggccacctgtcccgccataccaagatacacctgcgccaaaaagatgcggcccggggatcc

SEQ ID NO: 90 - DNA, 555 bp, synthetic construct
  source 1..555: mol_type = other DNA
ggggtacccgccgctatggctgagaggcccttccagtgtcgaatctgcatgcgtaacttcagttccgaggccgaccgctcccgccacatccgcacccacaccggcgagaagccttttgcctgtgacatttgtgggaggaaatttgccgaccgctccaacctgacccgccataccaagatacacacgggcagccaaaagcccttccagtgtcgaatctgcatgcgtaacttcagtcgctccgacaacctgtccgtgcacatccgcacccacaccggcgagaagccttttgcctgtgacatttgtgggaggaaatttgcccgccgcttcctgcgcaacaaccataccaagatacacacgggatctcagaagcccttccagtgtcgaatctgcatgcgtaacttcagttacgactacggccgctacacccacatccgcacccacaccggcgagaagccttttgcctgtgacatttgtgggaggaaatttgccgaccgctcccacctggcccgccataccaagatacacctgcgccaaaaagatgcggcccggggatcc

SEQ ID NO: 91 - DNA, 555 bp, synthetic construct
  source 1..555: mol_type = other DNA
ggggtacccgccgctatggctgagaggcccttccagtgtcgaatctgcatgcgtaacttcagtgaccgctcccacctgacccgccacatccgcacccacaccggcgagaagccttttgcctgtgacatttgtgggaggaaatttgcccagtccgcccacctgaaggcccataccaagatacacacgggcagccaaaagcccttccagtgtcgaatctgcatgcgtaacttcagtcgctcccactccctgctgcgccacatccgcacccacaccggcgagaagccttttgcctgtgacatttgtgggaggaaatttgcccgccgcgacaccctgctggaccataccaagatacacacgggatctcagaagcccttccagtgtcgaatctgcatgcgtaacttcagtgaccgctccaacctgtcccgccacatccgcacccacaccggcgagaagccttttgcctgtgacatttgtgggaggaaatttgccgacgccttcacccgcacccgccataccaagatacacctgcgccaaaaagatgcggcccggggatcc

SEQ ID NO: 92 - DNA, 555 bp, synthetic construct
  source 1..555: mol_type = other DNA
ggggtacccgccgctatggctgagaggcccttccagtgtcgaatctgcatgcgtaacttcagtcgctccgacgccatgtcccagcacatccgcacccacaccggcgagaagccttttgcctgtgacatttgtgggaggaaatttgcccgcaacgcctcccgcacccgccataccaagatacacacgggcagccaaaagcccttccagtgtcgaatctgcatgcgtaacttcagtcgctccgccgacctgacccgccacatccgcacccacaccggcgagaagccttttgcctgtgacatttgtgggaggaaatttgcccgctccgacgacctgacccgccataccaagatacacacgggatctcagaagcccttccagtgtcgaatctgcatgcgtaacttcagtcacgcctgttgtcgcaacatgcacatccgcacccacaccggcgagaagccttttgcctgtgacatttgtgggaggaaatttgcccgctccgccgacctgtcccgccataccaagatacacctgcgccaaaaagatgcggcccggggatcc

SEQ ID NO: 93 - protein, 301 aa, unidentified
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 94 - protein, 302 aa, synthetic construct
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 95 - protein, 215 aa, Mus musculus
MGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 96 - protein, 421 aa, Mus musculus
MGSRETPSSCSKTLETLDLETSDSSSPDADSPLEEQWLKSSPALKEDSVDVVLEDCKEPLSPSSPPTGREMIRYEVKVNRRSIEDICLCCGTLQVYTRHPLFEGGLCAPCKDKFLESLFLYDDDGHQSYCTICCSGGTLFICESPDCTRCYCFECVDILVGPGTSERINAMACWVCFLCLPFSRSGLLQRRKRWRHQLKAFHDQEGAGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 97 - RNA, 1831 nt, Mus musculus
  source 1..1831: mol_type = mRNA
aacagaaaggaaatcaaaaccacctgcctgcctcaccgcccagaaactcagcctttgggacagtaagacgctgagaggctgtgggcctcctccaatgctcagatgctgagaaggaagcattaggtgatgccaaaatgggcttcctgagaaacaccacctcttcctcatccccaaaggaggtttctgctcactggcctctcccactgtctcaggcagggttctgacgaccctgctgtcacacccgccatcccttggacgcagacccttctagccgattacatcaatgggttcccgggagacaccttcttcttgctctaagacccttgaaaccttggacctggagacttccgacagctctagccctgatgctgacagtcctctggaagagcaatggctgaaatcctccccagccctgaaggaggacagtgtggatgtggtactggaagactgcaaagagcctctgtccccctcctcgcctccgacaggcagagagatgatcaggtacgaagtcaaagtgaaccgacggagcattgaagacatctgcctctgctgtggaactctccaggtgtacactcggcaccccttgtttgagggagggttatgtgccccatgtaaggataagttcctggagtccctcttcctgtatgatgatgatggacaccagagttactgcaccatctgctgttccgggggtaccctgttcatctgtgagagccccgactgtaccagatgctactgtttcgagtgtgtggacatcctggtgggccccgggacctcagagaggatcaatgccatggcctgctgggtttgcttcctgtgcctgcccttctcacggagtggactgctgcagaggcgcaagaggtggcggcaccagctgaaggccttccatgatcaagagggagcgggccctatggagatatacaagacagtgtctgcatggaagagacagccagtgcgggtactgagcctttttagaaatattgataaagtactaaagagtttgggctttttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctctttagaaatgaatcaccataagatgaaagtctttcctagaaccagggcagatttcttcctaaggtctcttccctccacagttttctctggtttgctttcaggccttcgggtttctctcctgtttgattgccaggatgcctctgtgcagctcactttgcggggtgggaggtgcctacggctctgcacaagttcccggtgggataacctgccatgtttctctgaaactgtgtgtacctgttgtgaagtttttcaaatatatcataggattgttactggtaaaaaaaaaa

SEQ ID NO: 98 - protein, 543 aa, synthetic construct
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 99 - protein, 27 aa, synthetic construct
SSGNSNANSRGPSFSSGLVPLSLRGSH

SEQ ID NO: 100 - protein, 71 aa, Homo sapiens
DAKSLTAWSRTLVTFKDVFVDFTREEWKLLDTAQQIVYRNVMLENYKNLVSLGYQLTKPDVILRLEKGEEP

SEQ ID NO: 101 - protein, 7 aa, synthetic construct
PKKKRKV

SEQ ID NO: 102 - protein, 16 aa, synthetic construct
KRPAATKKAGQAKKKK

SEQ ID NO: 103 - protein, 9 aa, synthetic construct
PAAKRVKLD

SEQ ID NO: 104 - protein, 11 aa, synthetic construct
RQRRNELKRSP

SEQ ID NO: 105 - protein, 38 aa, synthetic construct
NQSSNFGPMKGGNFGGRSSGPYGGGGQYFAKPRNQGGY

SEQ ID NO: 106 - protein, 42 aa, synthetic construct
RMRIZFKNKGKDTAELRRRRVEVSVELRKAKKDEQILKRRNV

SEQ ID NO: 107 - protein, 8 aa, synthetic construct
VSRKRPRP

SEQ ID NO: 108 - protein, 8 aa, synthetic construct
PPKKARED

SEQ ID NO: 109 - protein, 8 aa, synthetic construct
PQPKKKPL

SEQ ID NO: 110 - protein, 12 aa, synthetic construct
SALIKKKKKMAP

SEQ ID NO: 111 - protein, 5 aa, synthetic construct
DRLRR

SEQ ID NO: 112 - protein, 7 aa, synthetic construct
PKQKKRK

SEQ ID NO: 113 - protein, 10 aa, synthetic construct
RKLKKKIKKL

SEQ ID NO: 114 - protein, 10 aa, synthetic construct
REKKKFLKRR

SEQ ID NO: 115 - protein, 20 aa, synthetic construct
KRKGDEVDGVDEVAKKKSKK

SEQ ID NO: 116 - protein, 17 aa, synthetic construct
RKCLQAGMNLEARKTKK

SEQ ID NO: 117 - protein, 5 aa, synthetic construct
  VARIANT >5: note = The amino acid at position 5 may be repeated one or more times
GSGGS

SEQ ID NO: 118 - protein, 6 aa, synthetic construct
  VARIANT >6: note = The amino acid at position 6 may be repeated one or more times
GGSGGS

SEQ ID NO: 119 - protein, 5 aa, synthetic construct
  VARIANT >5: note = The entire sequence of amino acids may be repeated one or more times
GGGGS

SEQ ID NO: 120 - protein, 4 aa, synthetic construct
  VARIANT >4: note = The amino acid at position 4 may be repeated one or more times
GGGS

SEQ ID NO: 121 - protein, 4 aa, synthetic construct
GGSG

SEQ ID NO: 122 - protein, 5 aa, synthetic construct
GGSGG

SEQ ID NO: 123 - protein, 5 aa, synthetic construct
GSGSG

SEQ ID NO: 124 - protein, 5 aa, synthetic construct
GSGGG

SEQ ID NO: 125 - protein, 5 aa, synthetic construct
GGGSG

SEQ ID NO: 126 - protein, 5 aa, synthetic construct
GSSSG

SEQ ID NO: 127 - protein, 7 aa, synthetic construct
GGSGGGS

SEQ ID NO: 128 - protein, 17 aa, synthetic construct
EASGSGRASPGIPGSTR

SEQ ID NO: 129 - protein, 9 aa, synthetic construct
YPYDVPDYA

SEQ ID NO: 130 - protein, 19 aa, synthetic construct
ATNFSLLKQAGDVEENPGP

SEQ ID NO: 131 - protein, 16 aa, synthetic construct
SGSETPGTSESATPES

SEQ ID NO: 132 - protein, 80 aa, synthetic construct
GGPSSGAPPPSGGSPAGSPTSTEEGTSESATPESGPGTSTEPSEGSAPGSPAGSPTSTEEGTSTEPSEGSAPGTSTEPSE

SEQ ID NO: 133 - protein, 16 aa, Drosophila melanogaster
RQIKIWFQNRRMKWKK

SEQ ID NO: 134 - protein, 11 aa, synthetic construct
YGRKKRRQRRR

SEQ ID NO: 135 - protein, 12 aa, synthetic construct
RRQRRTSKLMKR

SEQ ID NO: 136 - protein, 27 aa, synthetic construct
GWTLNSAGYLLGKINLKALAALAKKIL

SEQ ID NO: 137 - protein, 33 aa, synthetic construct
KALAWEAKLAKALAKALAKHLAKALAKALKCEA

SEQ ID NO: 138 - protein, 9 aa, synthetic construct
RKKRRQRRR

SEQ ID NO: 139 - protein, 8 aa, synthetic construct
RKKRRQRR

SEQ ID NO: 140 - protein, 11 aa, synthetic construct
YARAAARQARA

SEQ ID NO: 141 - protein, 11 aa, synthetic construct
THRLPRRRRRR

SEQ ID NO: 142 - protein, 11 aa, synthetic construct
GGRRARRRRRR

SEQ ID NO: 143 - RNA, 20 nt, synthetic construct
  source 1..20: mol_type = other RNA
gctgcgactcggcggagtcc